(19) United States
(12) Patent Application Publication
(10) Pub. No.: US 2019/0093081 A1
(43) Pub. Date: Mar. 28, 2019

(54) PLACENTA-DERIVED INTERMEDIATE NATURAL KILLER (PINK) CELLS FOR TREATMENT OF GLIOBLASTOMA

(71) Applicant: CELULARITY, INC., Warren, NJ (US)

(72) Inventors: Lin KANG, Basking Ridge, NJ (US); Xiaokui ZHANG, Livingston, NJ (US); William VAN DER TOUW, Hoboken, NJ (US); Robert J. HARIRI, Bernardsville, NJ (US); Shuyang HE, Martinsville, NJ (US)

(73) Assignee: CELULARITY, INC., Warren, NJ (US)

(21) Appl. No.: 16/147,525

(22) Filed: Sep. 28, 2018

Related U.S. Application Data
(60) Provisional application No. 62/564,735, filed on Sep. 28, 2017.

(51) Int. Cl. C12N 5/0783; A61P 35/00; A61K 35/17; C12N 5/071

(52) U.S. Cl. CPC C12N 5/0646; A61P 35/00; A61K 35/17; C12N 5/0697; C12N 2501/145; C12N 2501/2307; C12N 2501/2302; C12N 2501/999; C12N 2501/26; C12N 2501/2306; C12N 2501/2315

(57) ABSTRACT

Provided provided herein are methods of treating a subject having a brain tumor, e.g., a glioblastoma by administering to the subject an effective amount of a cell population comprising human placenta-derived natural killer cells. Also provided are methods of suppressing the growth of brain tumor cells comprising contacting the glioblastoma cells with an effective amount of a cell population comprising human placenta-derived natural killer cells. Further provided are compositions comprising subject an effective amount of a cell population comprising human placenta-derived natural killer cells for use in the treatment of a brain tumor in a subject or for use in the manufacture of a medicament for treatment of a brain tumor in a subject.

This application claims priority to U.S. Provisional Patent Application No. 62/564,735, filed Sep. 28, 2017, the disclosure of which is incorporated herein by reference in its entirety.

### 1. FIELD

Presented herein are methods of suppressing the growth or proliferation of brain tumor cells, e.g., glioblastoma cells, by contacting the tumor cells with natural killer (NK) cells derived from placenta (e.g., NK cells isolated from umbilical cord blood or placental perfusate or NK cells differentiated from CD34⁺ hematopoietic stem cells recovered from umbilical cord blood or placental perfusate). Specifically provided herein are methods of treating individuals having a brain tumor, such as a glioblastoma, by administering to the subject an effective amount of a cell population comprising human placenta-derived natural killer cells.

### 2. BACKGROUND

Placental perfusate comprises a collection of placental cells obtained by passage of a perfusion solution through the placental vasculature, and collection of the perfusion fluid from the vasculature, from the maternal surface of the placenta, or both. Methods of perfusing mammalian placentas are described, e.g., in U.S. Pat. Nos. 7,045,146 and 7,255,879. The population of placental cells obtained by perfusion is heterogenous, comprising hematopoietic (CD34⁺) cells, nucleated cells such as granulocytes, monocytes and macrophages, a small percentage (less than 1%) tissue culture substrate-adherent placental stem cells, and natural killer cells.

Natural killer (NK) cells are cytotoxic lymphocytes that constitute a major component of the innate immune system. NK cells do not express T-cell antigen receptors (TCR), CD3 or surface immunoglobulins (Ig) B cell receptor, but usually express the surface markers CD16 (FcγRIII) and CD56 in humans. NK cells are cytotoxic; small granules in their cytoplasm contain special proteins such as perforin and proteases known as granzymes. Upon release in close proximity to a cell slated for killing, perforin forms pores in the cell membrane of the target cell through which the granzymes and associated molecules can enter, inducing apoptosis. One granzyme, granzyme B (also known as granzyme 2 and cytotoxic T-lymphocyte-associated serine esterase 1), is a serine protease crucial for rapid induction of target cell apoptosis in the cell-mediated immune response.

NK cells are activated in response to interferons or macrophage-derived cytokines. Activated NK cells are referred to as lymphokine activated killer (LAK) cells. NK cells possess two types of surface receptors, labeled “activating receptors” and “inhibitory receptors,” that control the cells' cytotoxic activity.

Among other activities, NK cells play a role in the host rejection of tumors. Because cancer cells have reduced or no class I MHC expression, they can become targets of NK cells. Accumulating clinical data suggest that haploidentical transplantation of human NK cells isolated from PBMC or bone marrow mediate potent anti-leukemia effects without possessing detectable graft versus host disease (GVHD). See Ruggeri et al., Science 295:2097-2100 (2002)). Natural killer cells can become activated by cells lacking, or displaying reduced levels of, major histocompatibility complex (MHC) proteins. Activated and expanded NK cells and LAK cells have been used in both ex vivo therapy and in vivo treatment of patients having advanced cancer, with some success against bone marrow related diseases, such as leukemia; breast cancer; and certain types of lymphoma.

In spite of the advantageous properties of NK cells in killing tumor cells and virus-infected cells, they remain difficult to apply in immunotherapy, primarily due to the difficulty in maintaining their tumor-targeting and tumoricidal capabilities during culture and expansion. Thus, there is a need in the art to develop an efficient method to produce and expand natural killer cells that retain tumoricidal functions

Glioblastomas comprise ˜75% of all malignant brain tumors accounting for 12,390 estimated cases in 2017 (CBTRUS). The standard of care for glioblastoma includes tumor resection, radiation therapy, and temozolomide chemotherapy. Even so, however, the 5-year survival rate is less than 5%. Accordingly, there is an unmet need for new therapies for the treatment of glioblastomas.

### 3. SUMMARY

Provided herein are methods of suppressing the growth or proliferation brain tumor cells, e.g., glioblastoma cells, by contacting the tumor cells with natural killer (NK) cells derived from placenta (e.g., NK cells isolated from umbilical cord blood or placental perfusate or NK cells differentiated from CD34⁺ hematopoietic stem cells recovered from umbilical cord blood or placental perfusate). Specifically provided herein are methods of treating individuals having a brain tumor, such as a glioblastoma, by administering to the subject an effective amount of a cell population comprising human placenta-derived natural killer cells.

In one aspect, provided herein are methods of treating a subject having glioblastoma by administering to the subject an effective amount of a cell population comprising human placenta-derived natural killer cells.

In another aspect, provided herein are methods of treating a subject having a brain tumor by administering to the subject an effective amount of a cell population comprising human placenta-derived natural killer cells.

In another aspect, provided herein are methods of suppressing the growth of brain tumor cells comprising contacting the glioblastoma cells with an effective amount of a cell population comprising human placenta-derived natural killer cells.

In another aspect, provided herein are compositions comprising subject an effective amount of a cell population comprising human placenta-derived natural killer cells for use in the treatment of a brain tumor in a subject.

In another aspect, provided herein are uses of a composition comprising subject an effective amount of a cell population comprising human placenta-derived natural killer cells in the treatment of a brain tumor in a subject.

In another aspect, provided herein are uses of a composition comprising subject an effective amount of a cell population comprising human placenta-derived natural killer cells in the manufacture of a a brain tumor for treatment of glioblastoma in a subject.

In some preferred embodiments of the above aspects, the brain tumor is a glioplastoma.

In some embodiments of the above aspects, the human placenta-derived natural killer cells are derived from umbilical cord blood, placental perfusate, or combinations thereof. In preferred aspects, the human placenta-derived natural killer cells are derived from umbilical cord blood.

In some embodiments of the above aspects, the human placenta-derived natural killer cells are produced from hematopoietic stem cells. In preferred aspects, the hematopoietic stem cells are CD34+ hematopoietic stem cells.

In some embodiments of the above aspects, the human placenta-derived natural killer cells are produced by a method comprising the steps of: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking a stem cell mobilizing agent and LMWH, to produce a third population of cells. In preferred aspects, the third population of cells comprises natural killer cells that are CD56+, CD3−, CD16− or CD16+, and CD94+ or CD94−, and wherein at least 80% of the natural killer cells are viable.

In some embodiments of the above aspects, said Tpo is present in the first medium at a concentration of from 1 ng/mL to 50 ng/mL. In other embodiments, said Tpo is present in the first medium at a concentration of from 20 ng/mL to 30 ng/mL. In other embodiments, said Tpo is present in the first medium at a concentration of about 25 ng/mL.

In some embodiments of the above aspects, said IL-15 is present in said second medium at a concentration of from 1 ng/mL to 50 ng/mL. In other embodiments, said IL-15 is present in said second medium at a concentration of from 10 ng/mL to 30 ng/mL. In other embodiments, said IL-15 is present in said second medium at a concentration of about 20 ng/mL.

In some embodiments of the above aspects, said IL-2 is present in said third medium at a concentration of from 10 U/mL to 10,000 U/mL and said IL-15 is present in said third medium at a concentration of from 1 ng/mL to 50 ng/mL. In other embodiments, said IL-2 is present in said third medium at a concentration of from 300 U/mL to 3,000 U/mL and said IL-15 is present in said third medium at a concentration of from 10 ng/mL to 30 ng/mL. In other embodiments, said IL-2 is present in said third medium at a concentration of about 1,000 U/mL and said IL-15 is present in said third medium at a concentration of about 20 ng/mL.

In some embodiments of the above aspects, said Tpo, IL-2, and IL-15 are not comprised within an undefined component of the first medium, second medium or third medium. In some embodiments, said Tpo, IL-2, and IL-15 are not comprised within serum.

In some embodiments of the above aspects, said stem cell mobilizing agent is an aryl hydrocarbon receptor inhibitor. In some embodiments, said aryl hydrocarbon receptor inhibitor is StemRegenin-1 (SR-1) (4-(2-(2-(benzo[b]thiophen-3-yl)-9-isopropyl-9H-purin-6-ylamino)ethyl)phenol). In other embodiments, wherein said aryl hydrocarbon receptor inhibitor is resveratrol. In other embodiments, said aryl hydrocarbon receptor inhibitor is the compound CH223191 (1-Methyl-N-[2-methyl-4-[2-(2-methylphenyl)diazenyl]phenyl-1H-pyrazole-5-carboxamide]. In other embodiments, the stem cell mobilizing agent is a pyrimido(4,5-b)indole derivative. In some embodiments, said pyrimido(4,5-b)indole derivative has the chemical structure:

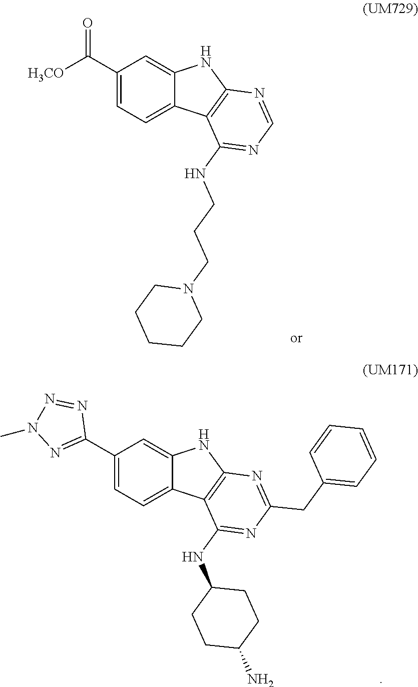 [SMILES: COC(=O)c1ccc2c(c1)[nH]c1ncnc(NCCCN3CCCCC3)c12 ; Cn1nnc(-c2ccc3c(c2)[nH]c2nc(Cc4ccccc4)nc(N[C@H]4CC[C@H](N)CC4)c23)n1]

In some embodiments of the above aspects, said first medium additionally comprises one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In some embodiments, said first medium comprises each of LMWH, Flt-3L, SCF, IL-6, IL-7, G-CSF, and GM-CSF. In some embodiments, in the first medium the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In some embodiments, in the first medium the LMWH is present in the first medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In some embodiments, in the first medium the LMWH is present in the first medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL.

In some embodiments of the above aspects, said second medium additionally comprises one or more of LMWH, Flt-3, SCF, IL-6, IL-7, G-CSF, and GM-CSF. In some embodiments, said second medium additionally comprises each of LMWH, Flt-3, SCF, IL-6, IL-7, G-CSF, and GM-CSF. In some embodiments, in the second medium the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In some embodiments, in the second medium the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In some embodiments, in the second medium the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL.

In some embodiments of the above aspects, said third medium additionally comprises one or more of SCF, IL-6, IL-7, G-CSF, or GM-CSF. In some embodiments, said third medium comprises each of SCF, IL-6, IL-7, G-CSF, and GM-CSF. In some embodiments, in the third medium the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In some embodiments, in the third medium the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In some embodiments, in the third medium the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL.

In some embodiments of the above aspects, said LMWH, Flt-3, SCF, IL-6, IL-7, G-CSF, and/or GM-CSF are not comprised within an undefined component of the first medium, second medium or third medium. In some embodiments, said LMWH, Flt-3, SCF, IL-6, IL-7, G-CSF, and/or GM-CSF are not comprised within serum.

In some embodiments of the above aspects, any of said first medium, second medium or third medium comprises human serum-AB. In some embodiments, any of said first medium, second medium or third medium comprises 1% to 20% human serum-AB. In some embodiments, any of said first medium, second medium or third medium comprises 5% to 15% human serum-AB. In some embodiments, any of said first medium, second medium or third medium comprises about 10% human serum-AB.

In some embodiments of the above aspects, any of said first medium, second medium or third medium comprises 2-mercaptoethanol.

In some embodiments of the above aspects, any of said first medium, second medium or third medium comprises gentamycin.

In some embodiments of the above aspects, said method comprises culturing the hematopoietic stem cells in the first medium for 7-13 days, for 8-12 days, or for about 10 days.

In some embodiments of the above aspects, said method comprises culturing said first population of cells in said second medium for 2-6 days, for 3-5 days, or for about 4 days.

In some embodiments of the above aspects, said method comprises culturing said second population of cells in said third medium for 10-30 days, for 15-25 days, or for about 21 days.

In some embodiments of the above aspects, said culturing in said first medium, second medium and third medium are all done under static culture conditions.

In some embodiments of the above aspects, said culturing in at least one of said first medium, second medium or third medium are done in a spinner flask.

In some embodiments of the above aspects, said culturing in said first medium and said second medium is done under static culture conditions, and said culturing in said third medium is done in a spinner flask.

In some embodiments of the above aspects, said hematopoietic cells are initially inoculated into said first medium from 1×10⁴ to 1×10⁵ cells/mL, or at about 3×10⁴ cells/mL.

In some embodiments of the above aspects, said first population of cells are initially inoculated into said second medium from 5×10⁴ to 5×10⁵ cells/mL, or at about 1×10⁵ cells/mL.

In some embodiments of the above aspects, said second population of cells is initially inoculated into said third medium from 1×10⁵ to 5×10⁶ cells/mL, or from 1×10⁵ to 1×10⁶ cells/mL, at about 5×10⁵ cells/mL, or at about 3×10⁵ cells/mL.

In some embodiments of the above aspects, said method produces at least 5000-, 10,000-, 50,000-, or 75,000-fold more natural killer cells as compared to the number of hematopoietic stem cells initially inoculated into said first medium.

In some embodiments of the above aspects, said method produces natural killer cells that comprise at least 20%, at least 40%, at least 60%, or at least 80% CD56+CD3− natural killer cells.

In some embodiments of the above aspects, said natural killer cells exhibit at least 20% cytotoxicity against K562 cells when said natural killer cells and said K562 cells are co-cultured in vitro at a ratio of 10:1. In some embodiments, said natural killer cells exhibit at least 35%, at least 45%, at least 60%, or at least 75% cytotoxicity against the K562 cells.

In some embodiments of the above aspects, viability of said natural killer cells is determined by 7-aminoactinomycin D (7AAD) staining. In some embodiments, viability of said natural killer cells is determined by annexin-V staining. In some embodiments, viability of said natural killer cells is determined by both 7-AAD staining and annexin-V staining. In some embodiments, viability of said natural killer cells is determined by trypan blue staining.

In some embodiments of the above aspects, the human placenta-derived natural killer cells are produced by a method additionally comprising the step of cryopreserving said population of cells after step (c). In some embodiments, said cryopreserved cell population is administered to the subject within about twenty-four, sixteen, twelve, eight, six, four, or two hours after thawing. In some embodiments, the human placenta-derived natural killer cells are not cryopreserved.

In some embodiments of the above aspects, the subject is a mammal. In some embodiments, the subject is a human.

In some embodiments of the above aspects, the treating further comprises administering to the subject an effective amount of an additional anti-cancer treatment. In some embodiments, the additional anti-cancer treatment is selected from the group consisting of radiation therapy, chemotherapy, antibody-based therapy, and combinations thereof. In some embodiments, the treating further comprises administering to the subject an effective amount of an anticonvulsant. In some embodiments, the treating further comprises administering to the subject an effective amount of a corticosteroid.

In some embodiments of the above aspects, said subject is administered about 1×10⁴, 3×10⁴, 1×10⁵, 3×10⁵, 1×10⁶, 3×10⁶, 1×10⁷, 3×10⁷, 1×10⁸, or 3×10⁸ natural killer cells per kilogram of the subject.

In some embodiments of the above aspects, the administration is intracranial, (IC), intracerebral ventricular (ICV), or intraveinous (IV).

In some embodiments of the above aspects, the treatment comprises administration of more than one dose of the cell population comprising human placenta-derived natural killer cells. In some embodiments, the treatment comprises administration of two, three, four, or more doses of the cell population comprising human placenta-derived natural killer cells.

In some embodiments of the above aspects, the natural killer cells are genetically modified.

In some embodiments of the above aspects, said contacting takes place in vitro, in vivo, or in a human individual. In some embodiments, said method comprises administering said natural killer cells to said individual.

### 4. BRIEF DESCRIPTION OF THE FIGURES

FIG. 1 shows that PNK cells exert cytotoxic activity against a range of GBM tumor cell lines. Shown is the average cytotoxic activity of PNK cells against the indicated tumor cell lines, K562, U-251, LN-18, U-87MG and U-118MG. The error bars represent the SD from the mean calculated from six different PNK donors.

FIG. 2 shows that PNK cells discriminate between healthy and tumor targets. Shown is the the average cytotoxic activity of PNK cells against the indicated targets, K562 cells and PBMCs from three different donors. The error bars represent the SD from the mean calculated from three different PNK donors.

FIG. 3 shows that PNK cells secrete IFN-γ in the presence of GBM tumor cell lines. Shown is the average IFN-γ secretion by PNK in the presence of GBM tumor cell lines: U-251 and U-87MG. The error bars represent the SD from the mean calculated from six different PNK donors.

FIGS. 4A and 4B show that, in presence of Unituxin®, the cytotoxicity of PNK cells against human glioblastoma cell lines U-251 (A) increased significantly, and the cytotoxicity of PNK cells against human glioblastoma cell lines U-87MG (B) likewise increased cytotoxicity, compared with that in presence of IgG1 control, at E:T ratio of 1:1.

FIGS. 5A, 5B and 5C show percent change from initial body weight after inoculation with U87-FLuc and PNK cells. PNK cells were injected IC (A, Groups 1 and 2), ICV (B, Groups 3 and 4) or IV (C, Groups 5 and 6). The data are presented as the mean±SEM; ** P<0.010.

FIGS. 6A, 6B and 6C show U87-Fluc cell proliferation in NSG mice. PNK cells were injected IC (A, Groups 1 and 2), ICV (B, Groups 3 and 4) or IV (C, Groups 5 and 6). The data are presented as the mean±SEM; ** P<0.010.

### 5. DETAILED DESCRIPTION

Provided herein are methods of suppressing growth or proliferation of tumor cells and methods of treating individuals having tumor cells using placental perfusate, placental perfusate cells, PINK cells, and/or combined NK cells, in combination with an antibody (e.g., an anti-GD2 antibody). Also provided herein are compositions comprising placental perfusate, placental perfusate cells, PINK cells, and/or combined NK cells, in combination with an antibody (e.g., an anti-GD2 antibody), and methods of using such compositions.

### 5.1. Definitions

As used herein, “combined NK cells” are NK cells, e.g., from matched umbilical cord and human placental perfusate, wherein placental perfusate is obtained from the same placenta as the cord blood. NK cells from both are isolated separately or at the same time, and combined.

As used herein, “PINK,” “PINK cells,” “placental intermediate NK cells,” “PNK cells,” or “placenta-derived intermediate NK cells” refers to NK cells that are obtained from human placenta, e.g., human placental perfusate or placental tissue that has been mechanically and/or enzymatically disrupted, or generated by a two-step expansion and differentiation method using hematopoietic stem cells that are recovered from human placenta, as exemplified in U.S. Pat. No. 8,926,964. PINK cells are CD56⁺ and CD16, e.g., as determined by flow cytometry, e.g., fluorescence-activated cell sorting using antibodies to CD56 and CD16. PINK cells are not obtained from umbilical cord blood or peripheral blood. In some specific embodiments, examples of PNK cells include PNK-007 cells.

As used herein, “placental perfusate” means perfusion solution that has been passed through at least part of a placenta, e.g., a human placenta, e.g., through the placental vasculature, including a plurality of cells collected by the perfusion solution during passage through the placenta.

As used herein, “placental perfusate cells” means nucleated cells, e.g., total nucleated cells, isolated from, or isolatable from, placental perfusate.

As used herein, “tumor cell suppression,” “suppression of tumor cell proliferation,” “suppressing growth of tumor cells,” and the like, refer to slowing the growth of a population of tumor cells, e.g., by killing one or more of the tumor cells in said population of tumor cells, for example, by contacting the population of tumor cells with PINK cells, a population of cells comprising PINK cells, combined NK cells, a population of cells comprising combined NK cells, human placental perfusate, or the like.

### 5.2. Placental Perfusate and Placental Perfusate Cells

Placental perfusate comprises a heterogeneous collection of cells. Typically, placental perfusate is depleted of erythrocytes prior to use. Such depletion can be carried out by known methods of separating red blood cells from nucleated blood cells. In certain embodiment, the perfusate or perfusate cells are cryopreserved. In certain other embodiments, the placental perfusate comprises, or the placental perfusate cells comprise, only fetal cells, or a combination of fetal cells and maternal cells.

Typically, placental perfusate from a single placental perfusion comprises about 100 million to about 500 million nucleated cells. In certain embodiments, the placental perfusate or perfusate cells comprise CD34⁺ cells, e.g., hematopoietic stem or progenitor cells. Such cells can, in a more specific embodiment, comprise CD34⁺CD45⁻ stem or progenitor cells, CD34⁺CD45⁺ stem or progenitor cells, myeloid progenitors, lymphoid progenitors, and/or erythroid progenitors. In other embodiments, placental perfusate and placental perfusate cells comprise adherent placental stem cells, e.g., CD34⁻ stem cells. In other embodiment, the placental perfusate and placental perfusate cells comprise, e.g., endothelial progenitor cells, osteoprogenitor cells, and natural killer cells. In certain embodiments, placental perfusate as collected from the placenta and depleted of erythrocytes, or perfusate cells isolated from such perfusate, comprise about 6-7% natural killer cells (CD3⁻, CD56⁺); about 21-22% T cells (CD3⁺); about 6-7% B cells (CD19⁺); about 1-2% endothelial progenitor cells (CD34⁺, CD31⁺); about 2-3% neural progenitor cells (nestin⁺); about 2-5% hematopoietic progenitor cells (CD34⁺); and about 0.5-1.5% adherent placental stem cells (e.g., CD34⁻, CD117⁻, CD105⁺ and CD44⁺), as determined, e.g. by flow cytometry, e.g., by FACS analysis.

### 5.3. Placental NK Cells

The isolation, characterization, and use of NK cells obtainable from placenta, e.g., from placental perfusate and/or from mechanically and/or enzymatically-disrupted placental tissue, are disclosed in U.S. Pat. No. 8,263,065, which is incorporated by reference herein in its entirety. Placental NK cells can also be generated by a two-step expansion and differentiation method using hematopoietic stem cells, as disclosed in U.S. Pat. No. 8,926,964, which is incorporated by reference herein in its entirety.

In a specific embodiment, the placental NK cells are “placental intermediate NK cells” or “PINK cells,” which are characterized as being CD56⁺CD16⁻, i.e., displaying the CD56 cellular marker and lacking the CD16 cellular marker, e.g., as determined by flow cytometry, e.g., fluorescence-activated cell sorting using antibodies against CD16 and CD56, as described above. In certain embodiments, the PINK cells are isolated from placenta. In some embodiments, the PINK cells are isolated from placental perfusate. In some embodiments, the PINK cells are isolated from placental perfusate cells. In some embodiments, the PINK cells are generated by a two-step expansion and differentiation method using hematopoietic stem cells. In certain embodiments, the hematopoietic stem cells are CD34⁺. In other embodiments, the hematopoietic stem cells are isolated from placenta. In yet other embodiments, the hematopoietic stem cells are isolated from placental perfusate. In some embodiments, the PINK cells are generated by a two-step expansion and differentiation method using CD34⁺ hematopoietic stem cells recovered from placenta. In some embodiments, the PINK cells are generated by a two-step expansion and differentiation method using CD34⁺ hematopoietic stem cells recovered from placental perfusate. In some embodiments, a plurality of NK cells comprises CD56⁺CD16⁻ PINK cells in combination with CD56⁺CD16⁺ NK cells. In more specific embodiments, the CD56⁺CD16⁺ NK cells can be isolated from placenta, or from another source, e.g., peripheral blood, umbilical cord blood, bone marrow, or the like. Thus, in various other embodiments, PINK cells can be combined with CD56⁺CD16⁺ NK cells, e.g., in ratios of, for example, about 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or about 9:1. As used in this context, “isolated” means that the cells have been removed from their normal environment, e.g., the placenta.

In certain embodiments, the PINK cells are CD3⁻.

In other embodiments, the PINK cells do not exhibit one or more cellular markers exhibited by fully mature NK cells (e.g., CD16), or exhibit such one or more markers at a detectably reduced level compared to fully mature NK cells, or exhibit one or more cellular markers associated with NK cell precursors but not fully mature NK cells. In a specific embodiment, a PINK cell provided herein expresses NKG2D, CD94 and/or NKp46 at a detectably lower level than a fully mature NK cell. In another specific embodiment, a plurality of PINK cells provided herein expresses, in total, NKG2D, CD94 and/or NKp46 at a detectably lower level than an equivalent number of fully mature NK cells.

In certain embodiments, PINK cells express one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, and/or hsa-miR-99a at a detectably higher level than peripheral blood NK cells.

In some embodiments, said PINK cells express one or more of aminopeptidase N protein, apolipoprotein E protein, atrophin-1 interacting protein 1, innexin inx-3 protein, integrin alpha-2 precursor protein, integrin beta-5 precursor, mast cell surface glycoprotein GP49B precursor protein, or ryanodine receptor 1 protein; and do not express one or more of fibroblast growth factor receptor 4 precursor protein, immunity-associated nucleotide 4-like protein, integrin alpha-L precursor protein, integrin beta 2 precursor protein, integrin beta 4 precursor protein, membrane-bound lytic murein transglycosylase D precursor protein, oxysterol binding protein-related protein 8, or perforin 1 precursor 1 protein.

In certain embodiments, the placental NK cells, e.g., PINK cells, have been expanded in culture. In certain other embodiments, the placental perfusate cells have been expanded in culture. In a specific embodiment, said placental perfusate cells have been expanded in the presence of a feeder layer and/or in the presence of at least one cytokine. In a more specific embodiment, said feeder layer comprises K562 cells or peripheral blood mononuclear cells. In another more specific embodiment, said at least one cytokine is interleukin-2.

In another embodiment, provided herein is an isolated plurality (e.g., population) of PINK cells. In another specific embodiment, the isolated population of cells is produced by CD56-microbead isolation of cells from placental perfusate. In various specific embodiments, the population comprises at least about 50%, 55%, 60%, 65%, 70%, 75%, 80%, 85%, 90%, 95%, 98% or at least about 99% PINK cells. In another embodiment, the plurality of PINK cells comprises, or consists of, PINK cells that have not been expanded; e.g., are as collected from placental perfusate. In another embodiment, the plurality of PINK cells comprises, or consists of, PINK cells that have been expanded. Methods of expanding NK cells have been described, e.g., in Ohno et al., U.S. Patent Application Publication No. 2003/0157713; see also Yssel et al., J Immunol. Methods 72(1):219-227 (1984) and Litwin et al., J. Exp. Med. 178(4):1321-1326 (1993) and the description of NK cell expansion in Example 1, below.

In other embodiments, the isolated plurality of PINK cells does not exhibit one or more cellular markers exhibited by fully mature NK cells (e.g., CD16), or exhibits such one or more markers at a detectably reduced level compared to fully mature NK cells, or exhibits one or more cellular markers associated with NK cell precursors but not associated with fully mature NK cells. In a specific embodiment, a PINK cell provided herein expresses NKG2D, CD94 and/or NKp46 at a detectably lower level than a fully mature NK cell. In another specific embodiment, a plurality of PINK cells provided herein expresses, in total, NKG2D, CD94 and/or NKp46 at a detectably lower level than an equivalent number of fully mature NK cells.

In certain specific embodiments, the population of PINK cells expresses one or more of the microRNAs hsa-miR-100, hsa-miR-127, hsa-miR-211, hsa-miR-302c, hsa-miR-326, hsa-miR-337, hsa-miR-497, hsa-miR-512-3p, hsa-miR-515-5p, hsa-miR-517b, hsa-miR-517c, hsa-miR-518a, hsa-miR-518e, hsa-miR-519d, hsa-miR-520g, hsa-miR-520h, hsa-miR-564, hsa-miR-566, hsa-miR-618, and/or hsa-miR-99a at a detectably higher level than peripheral blood NK cells. In another specific embodiment, the population of PINK cells expresses a detectably higher amount of granzyme B than an equivalent number of peripheral blood NK cells.

In other embodiments, the PINK cells provided herein have been expanded in culture. In specific embodiments, the PINK cells have been cultured, e.g., expanded in culture, for at least, about, or at most 1, 2, 3, 4, 5, 6, 7, 8, 9, 10, 11, 12, 13, 14, 15, 16, 17, 18, 19, 20, 21, 22, 23, 24, 25, 26, 27 or 28 days. In a specific embodiment, the PINK cells are cultured for about 21 days.

In another embodiment, provided herein is an isolated population of cells, e.g., placental cells, comprising PINK cells. In a specific embodiment, the isolated population of cells is total nucleated cells from placental perfusate, e.g., placental perfusate cells, comprising autologous, isolated PINK cells. In another specific embodiment, the population of cells is an isolated population of cells produced by CD56-microbead isolation of cells from placental perfusate. In various specific embodiments, the population comprises at least about 50%, 55%, 60%, 65%, 70%, 75%, 80%, 85%, 90%, 95%, 98% or at least about 99% PINK cells.

Because the post-partum placenta comprises tissue and cells from the fetus and from the mother placental perfusate, depending upon the method of collection, can comprise fetal cells only, or a substantial majority of fetal cells (e.g., greater than about 90%, 95%, 98% or 99%), or can comprise a mixture of fetal and maternal cells (e.g., the fetal cells comprise less than about 90%, 80%, 70%, 60%, or 50% of the total nucleated cells of the perfusate). In one embodiment, the PINK cells are derived only from fetal placental cells, e.g., cells obtained from closed-circuit perfusion of the placenta (see above) wherein the perfusion produces perfusate comprising a substantial majority, or only, fetal placental cells. In another embodiment, the PINK cells are derived from fetal and maternal cells, e.g., cells obtained by perfusion by the pan method (see above), wherein the perfusion produced perfusate comprising a mix of fetal and maternal placental cells. Thus, in one embodiment, provided herein is a population of placenta-derived intermediate NK cells, the substantial majority of which have the fetal genotype. In another embodiment, provided herein is a population of placenta-derived intermediate NK cells that comprise NK cells having the fetal genotype and NK cells having the maternal phenotype.

Also provided herein are populations of placenta-derived intermediate NK cells that comprise NK cells from a non-placental source. For example, in one embodiment, provided herein is population of PINK cells that also comprises NK cells from umbilical cord blood, peripheral blood, bone marrow, or a combination of two or more of the foregoing. The populations of NK cells comprising PINK cells and NK cells from a non-placental source can comprise the cells in, e.g., a ratio of about 1:10, 2:9, 3:8, 4:7:, 5:6, 6:5, 7:4, 8:3, 9:2, 10:1, 1:9, 1:8, 1:7, 1:6, 1:5, 1:4, 1:3, 1:2, 1:1, 2:1, 3:1, 4:1, 5:1, 6:1, 7:1, 8:1, 9:1,100:1, 95:5, 90:10, 85:15, 80:20, 75:25, 70:30, 65:35, 60:40, 55:45:50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, or about 1:100, or the like.

Further provided herein are combinations of umbilical cord blood and isolated PINK cells. In various embodiments, cord blood is combined with PINK cells at about 1×10⁴, 5×10⁴, 1×10⁵, 5×10⁵, 1×10⁶, 5×10⁶, 1×10⁷, 5×10⁷, 1×10⁸, or 5×10⁸, or more, PINK cells per milliliter of cord blood.

In one embodiment, PINK cells are collected by obtaining placental perfusate, then contacting the placental perfusate with a composition that specifically binds to CD56⁺ cells, e.g., an antibody against CD56, followed by isolating of CD56⁺ cells on the basis of said binding to form a population of CD56⁺ cells. The population of CD56⁺ cells comprises an isolated population of NK cells. In a specific embodiment, CD56⁺ cells are contacted with a composition that specifically binds to CD16⁺ cells, e.g., an antibody against CD16, and the CD16⁺ cells from the population of CD56⁺ cells. In another specific embodiment, CD3⁺ cells are also excluded from the population of CD56⁺ cells.

In one embodiment, PINK cells are obtained from placental perfusate as follows. Post-partum human placenta is exsanguinated and perfused, e.g., with about 200-800 mL of perfusion solution, through the placental vasculature only. In a specific embodiment, the placenta is drained of cord blood and flushed, e.g., with perfusion solution, through the placental vasculature to remove residual blood prior to said perfusing. The perfusate is collected and processed to remove any residual erythrocytes. NK cells in the total nucleated cells in the perfusate can be isolated on the basis of expression of CD56 and CD16. In certain embodiments, the isolation of PINK cells comprises isolation using an antibody to CD56, wherein the isolated cells are CD56⁺. In another embodiment, the isolation of PINK cells comprises isolation using an antibody to CD16, wherein the isolated cells are CD16⁻. In another embodiment, the isolation of PINK cells comprises isolation using an antibody to CD56, and exclusion of a plurality of non-PINK cells using an antibody to CD16, wherein the isolated cells comprise CD56⁺, CD16⁻ cells.

Cell separation can be accomplished by any method known in the art, e.g., fluorescence-activated cell sorting (FACS), or, preferably, magnetic cell sorting using microbeads conjugated with specific antibodies. Magnetic cell separation can be performed and automated using, e.g., an AUTOMACS™ Separator (Miltenyi).

In another aspect, the placental NK cells are isolated from a plurality of placental cells. In a specific embodiment, the placental cells are, or comprise, placental perfusate cells, e.g., total nucleated cells from placental perfusate. In another specific embodiment, said plurality of placental cells is, or comprises, placental cells obtained by mechanical and/or enzymatic digestion of placental tissue. In another embodiment, said isolating is performed using one or more antibodies. In a more specific embodiment, said one or more antibodies comprises one or more of antibodies to CD3, CD16 or CD56. In a more specific embodiment, said isolating comprises isolating CD56⁺ cells from CD56⁻ cells in said plurality of placental cells. In a more specific embodiment, said isolating comprises isolating CD56⁺, CD16⁻ placental cells, e.g., placental NK cells, e.g., PINK cells, from placental cells that are CD56⁻ or CD16⁺. In a more specific embodiment, said isolating comprises isolating CD56⁺, CD16⁻, CD3⁻ placental cells from placental cells that are CD56⁻, CD16⁺, or CD3⁺. In another embodiment, said method of isolating placental NK cells results in a population of placental cells that is at least 50%, 55%, 60%, 65%, 70%, 75%, 80%, 85%, 90%, 95%, 98% or at least 99% CD56⁺, CD16⁻ NK cells.

### 5.4. Placental NK Cells from Matched Perfusate and Cord Blood

Further provided herein are NK cells obtained, and obtainable from, combinations of matched units of placental perfusate and umbilical cord blood, referred to herein as combined NK cells. “Matched units,” as used herein, indicates that the NK cells are obtained from placental perfusate cells, and umbilical cord blood cells, wherein the umbilical cord blood cells are obtained from umbilical cord blood from the placenta from which the placental perfusate is obtained, i.e., the placental perfusate cells and umbilical cord blood cells, and thus the NK cells from each, are from the same individual.

In certain embodiments, the combined placental killer cells comprise only, or substantially only, NK cells that are CD56⁺ and CD16⁻. In certain other embodiments, the combined placental killer cells comprise NK cells that are CD56⁺ and CD16⁻, and NK cells that are CD56⁺ and CD16⁺. In certain specific embodiments, the combined placental killer cells comprise at least 50%, 60%, 70%, 80%, 90%, 95%, 96%, 97%, 98%, 99% or 99.5% CD56⁺CD16⁻ NK cells (PINK cells).

In one embodiment, the combined NK cells have not been cultured. In a specific embodiment, the combined NK cells comprise a detectably higher number of CD3 CD56⁺CD16⁻ NK cells than an equivalent number of NK cells from peripheral blood. In another specific embodiment, the combined NK cells comprise a detectably lower number of CD3⁻CD56⁺CD16⁻ NK cells than an equivalent number of NK cells from peripheral blood. In another specific embodiment, the combined NK cells comprise a detectably higher number of CD3⁻CD56⁺KIR2DL2/L3⁺ NK cells than an equivalent number of NK cells from peripheral blood. In another specific embodiment, the combined NK cells comprise a detectably lower number of CD3⁻CD56⁺ NKp46⁺ NK cells than an equivalent number of NK cells from peripheral blood. In another specific embodiment, the combined NK cells comprise a detectably lower number of CD3⁻CD56⁺ NKp30⁺ NK cells than an equivalent number of NK cells from peripheral blood. In another specific embodiment, the combined NK cells comprise a detectably lower number of CD3⁻CD56+2B4⁺ NK cells than an equivalent number of NK cells from peripheral blood. In another specific embodiment, the combined NK cells comprise a detectably lower number of CD3⁻CD56⁺CD94⁺ NK cells than an equivalent number of NK cells from peripheral blood.

In another embodiment, the combined NK cells have been cultured, e.g., for 21 days. In a specific embodiment, the combined NK cells comprise a detectably lower number of CD3⁻CD56⁺KIR2DL2/L3⁺ NK cells than an equivalent number of NK cells from peripheral blood. In another specific embodiment, the combined NK cells have not been cultured. In another specific embodiment, the combined NK cells comprise a detectably higher number of CD3⁻CD56⁺ NKp44⁺ NK cells than an equivalent number of NK cells from peripheral blood. In a specific embodiment, the combined NK cells comprise a detectably higher number of CD3⁻CD56⁺ NKp30⁺ NK cells than an equivalent number of NK cells from peripheral blood.

In another embodiment, the combined NK cells express a detectably higher amount of granzyme B than an equivalent number of peripheral blood NK cells.

Further provided herein are combinations of umbilical cord blood and combined NK cells. In various embodiments, cord blood is combined with combined NK cells at about 1×10⁴, 5×10⁴, 1×10⁵, 5×10⁵, 1×10⁶, 5×10⁶, 1×10⁷, 5×10⁷, 1×10⁸, 5×10⁸ combined NK cells per milliliter of cord blood.

### 5.5. Perfusate/Cell Combinations

In addition to placental perfusate, placental perfusate cells, combined NK cells, and placental NK cells, e.g., placental intermediate NK cells, provided herein are compositions comprising the perfusate or cells, for use in suppressing the proliferation of a tumor cell or plurality of tumor cells.

### 5.5.1. Combinations of Placental Perfusate, Perfusate Cells and Placenta-Derived Intermediate NK Cells

Further provided herein are compositions comprising combinations of the placental perfusate, placental perfusate cells, placental intermediate NK cells, and/or combined NK cells described in Sections 5.2, 5.3, or 5.4 above. In one embodiment, for example, provided herein is a volume of placental perfusate supplemented with a plurality of placental perfusate cells and/or a plurality of placental NK cells, e.g., placental intermediate NK cells, for example, obtained from placental perfusate cells or placental tissue mechanically or enzymatically disrupted. In specific embodiments, for example, each milliliter of placental perfusate is supplemented with about 1×10⁴, 5×10⁴, 1×10⁵, 5×10⁵, 1×10⁶, 5×10⁶, 1×10⁷, 5×10⁷, 1×10⁸, 5×10⁸ or more placental perfusate cells, placental intermediate NK cells, and/or combined NK cells. In another embodiment, a plurality of placental perfusate cells is supplemented with placental perfusate, placental intermediate NK cells, and/or combined NK cells. In another embodiment, a plurality of placental intermediate NK cells is supplemented with placental perfusate, placental perfusate cells, and/or combined NK cells. In certain embodiments, when perfusate is used for supplementation, the volume of perfusate is about, greater than about, or less than about, 50%, 45%, 40%, 35%, 30%, 25%, 20%, 15%, 10%, 8%, 6%, 4%, 2% or 1% of the total volume of cells (in solution) plus perfusate. In certain other embodiments, when placental perfusate cells are combined with a plurality of PINK cells and/or combined NK cells, the placental perfusate cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when PINK cells are combined with a plurality of placental perfusate cells and/or combined NK cells, the PINK cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when combined NK cells are combined with PINK cells and/or placental perfusate cells, the combined NK cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when PINK cells, combined NK cells or placental perfusate cells are used to supplement placental perfusate, the volume of solution (e.g., saline solution, culture medium or the like) in which the cells are suspended comprises about, greater than about, or less than about, 50%, 45%, 40%, 35%, 30%, 25%, 20%, 15%, 10%, 8%, 6%, 4%, 2% or 1% of the total volume of perfusate plus cells, where the PINK cells are suspended to about 1×10⁴, 5×10⁴, 1×10⁵, 5×10⁵, 1×10⁶, 5×10⁶, 1×10⁷, 5×10⁷, 1×10⁸, 5×10⁸ or more cells per milliliter prior to supplementation.

In other embodiments, any of the above combinations is, in turn, combined with umbilical cord blood or nucleated cells from umbilical cord blood.

Further provided herein is pooled placental perfusate that is obtained from two or more sources, e.g., two or more placentas, and combined, e.g., pooled. Such pooled perfusate can comprise approximately equal volumes of perfusate from each source, or can comprise different volumes from each source. The relative volumes from each source can be randomly selected, or can be based upon, e.g., a concentration or amount of one or more cellular factors, e.g., cytokines, growth factors, hormones, or the like; the number of placental cells in perfusate from each source; or other characteristics of the perfusate from each source. Perfusate from multiple perfusions of the same placenta can similarly be pooled.

Similarly, provided herein are placental perfusate cells, and placenta-derived intermediate NK cells, that are obtained from two or more sources, e.g., two or more placentas, and pooled. Such pooled cells can comprise approximately equal numbers of cells from the two or more sources, or different numbers of cells from one or more of the pooled sources. The relative numbers of cells from each source can be selected based on, e.g., the number of one or more specific cell types in the cells to be pooled, e.g., the number of CD34⁺ cells, the number of CD56⁺ cells, etc.

Pools can comprise, e.g., placental perfusate supplemented with placental perfusate cells; placental perfusate supplemented with placenta-derived intermediate NK (PINK) cells; placental perfusate supplemented with both placental perfusate cells and PINK cells; placental perfusate cells supplemented with placental perfusate; placental perfusate cells supplemented with PINK cells; placental perfusate cells supplemented with both placental perfusate and PINK cells; PINK cells supplemented with placental perfusate; PINK cells supplemented with placental perfusate cells; or PINK cells supplemented with both placental perfusate cells and placental perfusate.

Further provided herein are placental perfusate, placental perfusate cells, and placental intermediate NK cells, and pools of the same or combinations of the same, that have been assayed to determine the degree or amount of tumor suppression (that is, the potency) to be expected from, e.g., a given number of placental perfusate or PINK cells, or a given volume of perfusate. For example, an aliquot or sample number of cells is contacted with a known number of tumor cells under conditions in which the tumor cells would otherwise proliferate, and the rate of proliferation of the tumor cells in the presence of placental perfusate, perfusate cells, placental NK cells, or combinations thereof, over time (e.g., 1, 2, 3, 4, 5, 6, 7, 8, 9, or 10 weeks, or longer) is compared to the proliferation of an equivalent number of the tumor cells in the absence of perfusate, perfusate cells, placental NK cells, or combinations thereof. The potency of the placental perfusate, placental perfusate cells and/or PINK cells, or combinations or pools of the same, can be expressed, e.g., as the number of cells or volume of solution required to suppress tumor cell growth, e.g., by about 10%, 15%, 20%, 25%, 30%, 35%, 40%, 45%, 50%, or the like.

In certain embodiments, placental perfusate, placental perfusate cells, and PINK cells are provided as pharmaceutical grade administrable units. Such units can be provided in discrete volumes, e.g., 100 mL, 150 mL, 200 mL, 250 mL, 300 mL, 350 mL, 400 mL, 450 mL, 500 mL, or the like. Such units can be provided so as to contain a specified number of, e.g., placental perfusate cells, placental intermediate NK cells, or both, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10, 5×10⁸, 1×10⁹, 5×10⁹, 1×10¹⁰, 5×10¹⁰, 1×10¹¹ or more cells per unit. Such units can be provided to contain specified numbers of any two, or all three, of placental perfusate, placental perfusate cells, and/or PINK cells.

In the above combinations of placental perfusate, placental perfusate cells and/or PINK cells, any one, any two, or all three of the placental perfusate, placental perfusate cells and/or PINK cells can be autologous to a recipient (that is, obtained from the recipient), or homologous to a recipient (that is, obtained from at last one other individual from said recipient).

Any of the above combinations or pools of PINK cells, placental perfusate cells and/or placental perfusate can comprise CD56⁺CD16⁺ NK cells from, e.g., placental perfusate, peripheral blood, umbilical cord blood, bone marrow, or the like. In specific embodiments, the combinations comprise about, at least about, or at most about 1×10⁴, 5×10⁴, 1×10⁵, 5×10⁵, 1×10⁶, 5×10⁶ or more such NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. The CD56⁺CD16⁺ NK cells can be used as isolated from a natural source, or can be expanded prior to inclusion in one of the above combinations or pools. The CD56⁺CD16⁺ NK cells can be autologous (that is, obtained from the same individual as the placental perfusate, placental perfusate cells and/or PINK cells; or obtained from a recipient) or homologous (that is, derived from an individual different from the placental perfusate, placental perfusate cells and/or PINK cells; or from an individual that is not recipient).

Preferably, each unit is labeled to specify volume, number of cells, type of cells, whether the unit has been enriched for a particular type of cell, and/or potency of a given number of cells in the unit, or a given number of milliliters of the unit, causes a measurable suppression of proliferation of a particular type or types of tumor cell.

Also provided herein are compositions comprising placental intermediate NK cells, alone or in combination with placental perfusate cells and/or placental perfusate. Thus, in another aspect, provided herein is a composition comprising isolated CD56⁺, CD16⁻ NK cells, wherein said NK cells are isolated from placental perfusate, and wherein said NK cells comprise at least 50% of cells in the composition. In a specific embodiment, said NK cells comprise at least 80% of cells in the composition. In another specific embodiment, said composition comprises isolated CD56⁺, CD16⁺ NK cells. In a more specific embodiment, said CD56⁺, CD16⁺ NK cells are from a different individual than said CD56⁺, CD16⁻ NK cells. In another specific embodiment, said NK cells are from a single individual. In a more specific embodiment, said isolated NK cells comprise NK cells from at least two different individuals. In another specific embodiment, the composition comprises isolated placental perfusate. In a more specific embodiment, said placental perfusate is from the same individual as said NK cells. In another more specific embodiment, said placental perfusate comprises placental perfusate from a different individual than said NK cells. In another specific embodiment, the composition comprises placental perfusate cells. In a more specific embodiment, said placental perfusate cells are from the same individual as said NK cells. In another more specific embodiment, said placental perfusate cells are from a different individual than said NK cells. In another specific embodiment, the composition additionally comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals.

### 5.5.2. Compositions Comprising Adherent Placental Stem Cells

In other embodiments, the placental perfusate, plurality of placental perfusate cells, and/or plurality of PINK cells, or a combination or pool of any of the foregoing, is supplemented with adherent placental stem cells. Such stem cells are described, e.g, in Hariri U.S. Pat. Nos. 7,045,148 and 7,255,879. Adherent placental stem cells are not trophoblasts.

The placental perfusate, plurality of placental perfusate cells, and/or plurality of PINK cells, or a combination or pool of any of the foregoing can be supplemented with, e.g., 1×10⁴, 5×10⁴, 1×10⁵, 5×10⁵, 1×10⁶, 5×10⁶, 1×10⁷, 5×10⁷, 1×10, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁵, 5×10⁵, 1×10⁹, 5×10⁹, 1×10¹⁰, 5×10¹⁰, 1×10¹¹ or more adherent placental cells. The adherent placental stem cells in the combinations can be, e.g., adherent placental stem cells that have been cultured for, e.g., 1, 2, 3, 4, 5, 6, 7, 8, 9, 10, 12, 14, 16, 18, 20, 22, 24, 26, 28, 30, 32, 34, 36, 38, or 40 population doublings, or more.

Adherent placental stem cells, when cultured in primary cultures or in cell culture, adhere to the tissue culture substrate, e.g., tissue culture container surface (e.g., tissue culture plastic). Adherent placental stem cells in culture assume a generally fibroblastoid, stellate appearance, with a number of cytoplasmic processes extending from the central cell body. Adherent placental stem cells are, however, morphologically distinguishable from fibroblasts cultured under the same conditions, as the placental stem cells exhibit a greater number of such processes than do fibroblasts. Morphologically, placental stem cells are also distinguishable from hematopoietic stem cells, which generally assume a more rounded, or cobblestone, morphology in culture.

Adherent placental stem cells, and populations of placental stem cells, useful in the compositions and methods provided herein, express a plurality of markers that can be used to identify and/or isolate the stem cells, or populations of cells that comprise the stem cells. The adherent placental stem cells, and adherent stem cell populations useful in the compositions and methods provided herein include stem cells and stem cell-containing cell populations obtained directly from the placenta, or any part thereof (e.g., amnion, chorion, amnion-chorion plate, placental cotyledons, umbilical cord, and the like). The adherent placental stem cell population, in one embodiment, is a population (that is, two or more) of adherent placental stem cells in culture, e.g., a population in a container, e.g., a bag.

Adherent placental stem cells generally express the markers CD73, CD105, CD200, HLA-G, and/or OCT-4, and do not express CD34, CD38, or CD45. Adherent placental stem cells can also express HLA-ABC (MHC-1) and HLA-DR. These markers can be used to identify adherent placental stem cells, and to distinguish placental stem cells from other stem cell types. Because the placental stem cells can express CD73 and CD105, they can have mesenchymal stem cell-like characteristics. However, because the adherent placental stem cells can express CD200 and HLA-G, a fetal-specific marker, they can be distinguished from mesenchymal stem cells, e.g., bone marrow-derived mesenchymal stem cells, which express neither CD200 nor HLA-G. In the same manner, the lack of expression of CD34, CD38 and/or CD45 identifies the adherent placental stem cells as non-hematopoietic stem cells.

In one embodiment, the adherent placental stem cells are CD200⁺, HLA-G⁺, wherein the stem cells detectably suppress cancer cell proliferation or tumor growth. In a specific embodiment, said adherent stem cells are also CD73⁺ and CD105⁺. In another specific embodiment, said adherent stem cells are also CD34⁻, CD38⁻ or CD45⁻. In a more specific embodiment, said adherent stem cells are also CD34⁻, CD38⁻, CD45⁻, CD73⁺ and CD105⁺. In another embodiment, said adherent stem cells produce one or more embryoid-like bodies when cultured under conditions that allow the formation of embryoid-like bodies.

In another embodiment, the adherent placental stem cells are CD73⁺, CD105⁺, CD200⁺, wherein said stem cells detectably suppress cancer cell proliferation or tumor growth. In a specific embodiment of said populations, said adherent stem cells are HLA-G⁺. In another specific embodiment, said adherent stem cells are CD34⁻, CD38⁻ or CD45⁻. In another specific embodiment, said adherent stem cells are CD34⁻, CD38⁻ and CD45⁻. In a more specific embodiment, said adherent stem cells are CD34⁻, CD38⁻, CD45⁻, and HLA-G⁺. In another specific embodiment, said adherent placental stem cells produce one or more embryoid-like bodies when cultured under conditions that allow the formation of embryoid-like bodies.

In another embodiment, the adherent placental stem cells are CD200⁺, OCT-4⁺, wherein said stem cells detectably suppress cancer cell proliferation or tumor growth. In a specific embodiment, said adherent stem cells are CD73⁺ and CD105⁺. In another specific embodiment, said adherent stem cells are HLA-G⁺. In another specific embodiment, said adherent stem cells are CD34⁻, CD38⁻ and CD45⁻. In a more specific embodiment, said adherent stem cells are CD34⁻, CD38⁻, CD45⁻, CD73⁺, CD105⁺ and HLA-G⁺. In another specific embodiment, the adherent placental stem cells produce one or more embryoid-like bodies when cultured under conditions that allow the formation of embryoid-like bodies.

In another embodiment, the adherent placental stem cells are CD73⁺, CD105⁺ and HLA-G⁺, wherein said adherent stem cells detectably suppress cancer cell proliferation or tumor growth. In a specific embodiment of the above plurality, said adherent stem cells are also CD34⁻, CD38⁻ or CD45⁻. In another specific embodiment, said adherent stem cells are also CD34⁻, CD38⁻ and CD45⁻. In another specific embodiment, said adherent stem cells are also OCT-4⁺. In another specific embodiment, said adherent stem cells are also CD200⁺. In a more specific embodiment, said adherent stem cells are also CD34⁻, CD38⁻, CD45⁻, OCT-4⁺ and CD200⁺.

In another embodiment, the adherent placental stem cells are CD73⁺, CD105⁺ stem cells, wherein said stem cells produce one or more embryoid-like bodies under conditions that allow formation of embryoid-like bodies, and wherein said adherent stem cells detectably suppress cancer cell proliferation or tumor growth. In a specific embodiment, said adherent stem cells are also CD34⁻, CD38⁻ or CD45⁻. In another specific embodiment, said adherent stem cells are also CD34⁻, CD38⁻ and CD45⁻. In another specific embodiment, said adherent stem cells are also OCT-4⁺. In a more specific embodiment, said adherent stem cells are also OCT-4⁺, CD34⁻, CD38⁻ and CD45⁻.

In another embodiment, the adherent placental stem cells are OCT-4⁺ stem cells, wherein said adherent placental stem cells produce one or more embryoid-like bodies when cultured under conditions that allow the formation of embryoid-like bodies, and wherein said stem cells have been identified as detectably suppressing cancer cell proliferation or tumor growth.

In various embodiments, at least 10%, at least 20%, at least 30%, at least 40%, at least 50% at least 60%, at least 70%, at least 80%, at least 90%, or at least 95% of said isolated placental cells are OCT4⁺ stem cells. In a specific embodiment of the above populations, said stem cells are CD73⁺ and CD105⁺. In another specific embodiment, said stem cells are CD34⁻, CD38⁻, or CD45⁻. In another specific embodiment, said stem cells are CD200⁺. In a more specific embodiment, said stem cells are CD73⁺, CD105⁺, CD200⁺, CD34⁻, CD38⁻, and CD45⁻. In another specific embodiment, said population has been expanded, for example, passaged at least once, at least three times, at least five times, at least 10 times, at least 15 times, or at least 20 times.

In a more specific embodiment of any of the above embodiments, the adherent placental cells express ABC-p (a placenta-specific ABC transporter protein; see, e.g., Allikmets et al., Cancer Res. 58(23):5337-9 (1998)).

In another embodiment, the adherent placental stem cells are CD29⁺, CD44⁺, CD73⁺, CD90⁺, CD105⁺, CD200⁺, CD34⁻ and CD133⁻. In another embodiment, the adherent placental stem cells, the placental stem cells constitutively secrete IL-6, IL-8 and monocyte chemoattractant protein (MCP-1).

Each of the above-referenced placental stem cells can comprise placental stem cells obtained and isolated directly from a mammalian placenta, or placental stem cells that have been cultured and passaged at least 1, 2, 3, 4, 5, 6, 7, 8, 9, 10, 12, 14, 16, 18, 20, 25, 30 or more times, or a combination thereof. Tumor cell suppressive pluralities of the adherent placental stem cells described above can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental stem cells.

### 5.5.3. Compositions Comprising Placental Stem Cell Conditioned Media

Also provided herein is the use of a tumor-suppressive composition comprising PINK cells, placental perfusate and/or placental perfusate, and additionally conditioned medium. Adherent placental stem cells, placental perfusate cells and/or placental intermediate NK cells can be used to produce conditioned medium that is tumor cell suppressive, that is, medium comprising one or more biomolecules secreted or excreted by the stem cells that have a detectable tumor cell suppressive effect on a plurality of one or more types of immune cells. In various embodiments, the conditioned medium comprises medium in which placental cells (e.g., stem cells, placental perfusate cells, PINK cells) have grown for at least 1, 2, 3, 4, 5, 6, 7, 8, 9, 10, 11, 12, 13, 14 or more days. In other embodiments, the conditioned medium comprises medium in which placental cells have grown to at least 30%, 40%, 50%, 60%, 70%, 80%, 90% confluence, or up to 100% confluence. Such conditioned medium can be used to support the culture of a separate population of placental cells, or cells of another kind. In another embodiment, the conditioned medium provided herein comprises medium in which adherent placental stem cells and non-placental stem cells have been cultured.

Such conditioned medium can be combined with any of, or any combination of, placental perfusate, placental perfusate cells, and/or placental intermediate NK cells to form a tumor cell suppressive composition. In certain embodiments, the composition comprises less than half conditioned medium by volume, e.g., about, or less than about, 50%, 45%, 40%, 35%, 30%, 25%, 20%, 15%, 10%, 5%, 4%, 3%, 2%, or 1% by volume.

Thus, in one embodiment, provided herein is a composition comprising culture medium from a culture of placental stem cells, wherein said placental stem cells (a) adhere to a substrate; (b) express CD200 and HLA-G, or express CD73, CD105, and CD200, or express CD200 and OCT-4, or express CD73, CD105, and HLA-G, or express CD73 and CD105 and facilitate the formation of one or more embryoid-like bodies in a population of placental cells that comprise the placental stem cells, when said population is cultured under conditions that allow formation of embryoid-like bodies, or express OCT-4 and facilitate the formation of one or more embryoid-like bodies in a population of placental cells that comprise the placental stem cells when said population is cultured under conditions that allow formation of embryoid-like bodies; and (c) detectably suppress the growth or proliferation of a tumor cell or population of tumor cells. In a specific embodiment, the composition further comprises a plurality of said placental stem cells. In another specific embodiment, the composition comprises a plurality of non-placental cells. In a more specific embodiment, said non-placental cells comprise CD34+ cells, e.g., hematopoietic progenitor cells, such as peripheral blood hematopoietic progenitor cells, cord blood hematopoietic progenitor cells, or placental blood hematopoietic progenitor cells. The non-placental cells can also comprise other stem cells, such as mesenchymal stem cells, e.g., bone marrow-derived mesenchymal stem cells. The non-placental cells can also be one or more types of adult cells or cell lines. In another specific embodiment, the composition comprises an anti-proliferative agent, e.g., an anti-MIP-1α or anti-MIP-1β antibody.

In a specific embodiment, placental cell-conditioned culture medium or supernatant is obtained from a plurality of placental stem cells co-cultured with a plurality of tumor cells at a ratio of about 1:1, about 2:1, about 3:1, about 4:1, or about 5:1 placental stem cells to tumor cells. For example, the conditioned culture medium or supernatant can be obtained from a culture comprising about 1×10⁵ placental stem cells, about 1×10⁶ placental stem cells, about 1×10⁷ placental stem cells, or about 1×10⁸ placental stem cells, or more. In another specific embodiment, the conditioned culture medium or supernatant is obtained from a co-culture comprising about 1×10⁵ to about 5×10⁵ placental stem cells and about 1×10⁵ tumor cells; about 1×10⁶ to about 5×10⁶ placental stem cells and about 1×10⁶ tumor cells; about 1×10⁷ to about 5×10⁷ placental stem cells and about 1×10⁷ tumor cells; or about 1×10⁸ to about 5×10⁸ placental stem cells and about 1×10⁸ tumor cells.

In a specific embodiment, the conditioned medium suitable for administration to a 70 kg individual comprises supernatant conditioned by about 70 million placental stem cells in about 200 mL culture medium.

Conditioned medium can be condensed to prepare an administrable pharmaceutical-grade product. For example, conditioned medium can be condensed to about 90%, 80%, 70%, 60%, 50%, 40%, 30%, 20%, 10% or more by removal of water, e.g., by evaporation, lyophilization, or the like. In a specific embodiment, for example, 200 mL conditioned medium from about 70 million placental stem cells can be condensed to a volume of about 180 mL, 160 mL, 140 mL, 120 mL, 100 mL, 80 mL, 60 mL, 40 mL, 20 mL or less. The conditioned medium can also be substantially dried, e.g., to a powder, e.g., by evaporation, lyophilization or the like.

### 5.6. Placenta-Derived Natural Killer Cells

### 5.6.1. Hematopoietic Cells

Hematopoietic cells useful in the methods disclosed herein can be any hematopoietic cells able to differentiate into NK cells, e.g., precursor cells, hematopoietic progenitor cells, hematopoietic stem cells, or the like. Hematopoietic cells can be obtained from tissue sources such as, e.g., bone marrow, cord blood, placental blood, peripheral blood, liver or the like, or combinations thereof. Hematopoietic cells can be obtained from placenta. In a specific embodiment, the hematopoietic cells are obtained from placental perfusate. In one embodiment, the hematopoietic cells are not obtained from umbilical cord blood. In one embodiment, the hematopoietic cells are not obtained from peripheral blood. Hematopoietic cells from placental perfusate can comprise a mixture of fetal and maternal hematopoietic cells, e.g., a mixture in which maternal cells comprise greater than 5% of the total number of hematopoietic cells. In certain embodiments, hematopoietic cells from placental perfusate comprise at least about 90%, 95%, 98%, 99% or 99.5% fetal cells.

In another specific embodiment, the hematopoietic cells, e.g., hematopoietic stem cells or progenitor cells, from which the NK cell populations produced using a three-stage method described herein are produced, are obtained from placental perfusate, umbilical cord blood, fetal liver, mobilized peripheral blood, or bone marrow. In another specific embodiment, the hematopoietic cells, e.g., hematopoietic stem cells or progenitor cells, from which the NK cell populations produced using a three-stage method described herein are produced, are combined cells from placental perfusate and cord blood, e.g., cord blood from the same placenta as the perfusate. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45:50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%: 15%).

In certain embodiments, the cord blood and placental perfusate are combined at a ratio of 100:1, 95:5, 90:10, 85:15, 80:20, 75:25, 70:30, 65:35, 60:40, 55:45:50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In another specific embodiment, the hematopoietic cells, e.g., hematopoietic stem cells or progenitor cells from which said NK cell populations produced using a three-stage method described herein are produced, are from both umbilical cord blood and placental perfusate, but wherein said umbilical cord blood is isolated from a placenta other than the placenta from which said placental perfusate is obtained.

In certain embodiments, the hematopoietic cells are CD34+ cells. In specific embodiments, the hematopoietic cells useful in the methods disclosed herein are CD34+CD38+ or CD34+CD38−. In a more specific embodiment, the hematopoietic cells are CD34+CD38−Lin−. In another specific embodiment, the hematopoietic cells are one or more of CD2−, CD3−, CD11b−, CD11c−, CD14−, CD16−, CD19−, CD24−, CD56−, CD66b− and/or glycophorin A−. In another specific embodiment, the hematopoietic cells are CD2−, CD3−, CD11b−, CD11c−, CD14−, CD16−, CD19−, CD24−, CD56−, CD66b− and glycophorin A−. In another more specific embodiment, the hematopoietic cells are CD34+CD38−CD33−CD117−. In another more specific embodiment, the hematopoietic cells are CD34+CD38−CD33−CD117−CD235−CD36−.

In another embodiment, the hematopoietic cells are CD45+. In another specific embodiment, the hematopoietic cells are CD34+CD45+. In another embodiment, the hematopoietic cell is Thy-1+. In a specific embodiment, the hematopoietic cell is CD34+Thy-1+. In another embodiment, the hematopoietic cells are CD133+. In specific embodiments, the hematopoietic cells are CD34+CD133+ or CD133+Thy-1+. In another specific embodiment, the CD34+ hematopoietic cells are CXCR4+. In another specific embodiment, the CD34+ hematopoietic cells are CXCR4−. In another embodiment, the hematopoietic cells are positive for KDR (vascular growth factor receptor 2). In specific embodiments, the hematopoietic cells are CD34+KDR+, CD133+KDR+ or Thy-1+KDR+. In certain other embodiments, the hematopoietic cells are positive for aldehyde dehydrogenase (ALDH+), e.g., the cells are CD34+ALDH+.

In certain embodiments, the hematopoietic cells are CD34−.

The hematopoietic cells can also lack certain markers that indicate lineage commitment, or a lack of developmental naiveté. For example, in another embodiment, the hematopoietic cells are HLA-DR−. In specific embodiments, the hematopoietic cells are CD34+HLA-DR−, CD133+HLA-DR−, Thy-1+HLA-DR− or ALDH+HLA-DR− In another embodiment, the hematopoietic cells are negative for one or more, or all, of lineage markers CD2, CD3, CD11b, CD11c, CD14, CD16, CD19, CD24, CD56, CD66b and glycophorin A.

Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers is discussed in detail below.

Hematopoietic cells used in the methods provided herein can be a substantially homogeneous population, e.g., a population comprising at least about 95%, at least about 98% or at least about 99% hematopoietic cells from a single tissue source, or a population comprising hematopoietic cells exhibiting the same hematopoietic cell-associated cellular markers. For example, in various embodiments, the hematopoietic cells can comprise at least about 95%, 98% or 99% hematopoietic cells from bone marrow, cord blood, placental blood, peripheral blood, or placenta, e.g., placenta perfusate.

Hematopoietic cells used in the methods provided herein can be obtained from a single individual, e.g., from a single placenta, or from a plurality of individuals, e.g., can be pooled. Where the hematopoietic cells are obtained from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like.

Hematopoietic cells used in the methods disclosed herein can, in certain embodiments, comprise hematopoietic cells from two or more tissue sources. For example, in certain embodiments, when hematopoietic cells from two or more sources are combined for use in the methods herein, a plurality of the hematopoietic cells used to produce natural killer cells using a three-stage method described herein comprise hematopoietic cells from placenta, e.g., placenta perfusate. In various embodiments, the hematopoietic cells used to produce NK cell populations produced using a three-stage method described herein, comprise hematopoietic cells from placenta and from cord blood; from placenta and peripheral blood; from placenta and placental blood, or placenta and bone marrow. In one embodiment, the hematopoietic cells comprise hematopoietic cells from placental perfusate in combination with hematopoietic cells from cord blood, wherein the cord blood and placenta are from the same individual, i.e., wherein the perfusate and cord blood are matched. In embodiments in which the hematopoietic cells comprise hematopoietic cells from two tissue sources, the hematopoietic cells from the sources can be combined in a ratio of, for example, 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or 9:1.

### 5.6.2. Placental Hematopoietic Stem Cells

In certain embodiments, the hematopoietic cells used in the methods provided herein are placental hematopoietic cells. In one embodiment, placental hematopoietic cells are CD34+. In a specific embodiment, the placental hematopoietic cells are predominantly (e.g., at least about 50%, 55%, 60%, 65%, 70%, 75%, 80%, 85%, 90%, 95% or 98%) CD34+CD38− cells. In another specific embodiment, the placental hematopoietic cells are predominantly (e.g., at least about 50%, 55%, 60%, 65%, 70%, 75%, 80%, 85%, 90%, 95% or 98%) CD34+CD38+ cells. Placental hematopoietic cells can be obtained from a post-partum mammalian (e.g., human) placenta by any means known to those of skill in the art, e.g., by perfusion.

In another embodiment, the placental hematopoietic cell is CD45−. In a specific embodiment, the hematopoietic cell is CD34+CD45−. In another specific embodiment, the placental hematopoietic cells are CD34+CD45+.

### 5.6.3. Production of Natural Killer Cells and Natural Killer Cell Populations

Production of NK cells and NK cell populations by the present methods comprises expanding a population of hematopoietic cells. During cell expansion, a plurality of hematopoietic cells within the hematopoietic cell population differentiate into NK cells. In one aspect, provided herein is a method of producing NK cells comprising culturing hematopoietic stem cells or progenitor cells, e.g., CD34+ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, and wherein at least 70%, for example 80%, of the natural killer cells are viable with certain embodiments, such natural killer cells comprise natural killer cells that are CD16−. In certain embodiments, such natural killer cells comprise natural killer cells that are CD94+. In certain embodiments, such natural killer cells comprise natural killer cells that are CD94+ or CD16+. In certain embodiments, such natural killer cells comprise natural killer cells that are CD94− or CD16−. In certain embodiments, such natural killer cells comprise natural killer cells that are CD94+ and CD16+. In certain embodiments, such natural killer cells comprise natural killer cells that are CD94− and CD16−

### 5.6.4. Production of NK Cell Populations Using a Three-Stage Method

In one embodiment, provided herein is a three-stage method of producing NK cell populations. In certain embodiments, the method of expansion and differentiation of the hematopoietic cells, as described herein, to produce NK cell populations according to a three-stage method described herein comprises maintaining the cell population comprising said hematopoietic cells at between about 2×104 and about 6×106 cells per milliliter. In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×105 cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In a certain embodiment, the three-stage method comprises a first stage (“stage 1”) comprising culturing hematopoietic stem cells or progenitor cells, e.g., CD34+ stem cells or progenitor cells, in a first medium for a specified time period, e.g., as described herein, to produce a first population of cells. In certain embodiments, the first medium comprises a stem cell mobilizing agent and thrombopoietin (Tpo). In certain embodiments, the first medium comprises in addition to a stem cell mobilizing agent and Tpo, one or more of LMWH, Flt-3L, SCF, IL-6, IL-7, G-CSF, and GM-CSF. In a specific embodiment, the first medium comprises each of the first medium comprises in addition to a stem cell mobilizing agent and Tpo, each of LMWH, Flt-3L, SCF, IL-6, IL-7, G-CSF, and GM-CSF. In a specific embodiment, the first medium lacks added LMWH. In a specific embodiment, the first medium lacks added desulphated glycosaminoglycans. In a specific embodiment, the first medium lacks LMWH. In a specific embodiment, the first medium lacks desulphated glycosaminoglycans. In a specific embodiment, the first medium comprises in addition to a stem cell mobilizing agent and Tpo, each of Flt-3L, SCF, IL-6, IL-7, G-CSF, and GM-CSF.

In certain embodiments, subsequently, in “stage 2” said cells are cultured in a second medium for a specified time period, e.g., as described herein, to produce a second population of cells. In certain embodiments, the second medium comprises a stem cell mobilizing agent and interleukin-15 (IL-15), and lacks Tpo. In certain embodiments, the second medium comprises, in addition to a stem cell mobilizing agent and IL-15, one or more of LMWH, Flt-3, SCF, IL-6, IL-7, G-CSF, and GM-CSF. In certain embodiments, the second medium comprises, in addition to a stem cell mobilizing agent and IL-15, each of LMWH, Flt-3, SCF, IL-6, IL-7, G-CSF, and GM-CSF. In a specific embodiment, the second medium lacks added LMWH. In a specific embodiment, the second medium lacks added desulphated glycosaminoglycans. In a specific embodiment, the second medium lacks LMWH. In a specific embodiment, the second medium lacks desulphated glycosaminoglycans. In certain embodiments, the second medium comprises, in addition to a stem cell mobilizing agent and IL-15, each of Flt-3, SCF, IL-6, IL-7, G-CSF, and GM-CSF.

In certain embodiments, subsequently, in “stage 3” said cells are cultured in a third medium for a specified time period, e.g., as described herein, to produce a third population of cell, e.g., natural killer cells. In certain embodiments, the third medium comprises IL-2 and IL-15, and lacks a stem cell mobilizing agent and LMWH. In certain embodiments, the third medium comprises in addition to IL-2 and IL-15, one or more of SCF, IL-6, IL-7, G-CSF, and GM-CSF. In certain embodiments, the third medium comprises in addition to IL-2 and IL-15, each of SCF, IL-6, IL-7, G-CSF, and GM-CSF. In specific embodiments, the third medium lacks desulphated glycosaminoglycans. In specific embodiments, the third medium lacks added desulphated glycosaminoglycans.

In a specific embodiment, the three-stage method is used to produce NK cell populations. In certain embodiments, the three-stage method is conducted in the absence of stromal feeder cell support. In certain embodiments, the three-stage method is conducted in the absence of exogenously added steroids (e.g., cortisone, hydrocortisone, or derivatives thereof).

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In certain aspects, the first medium used in the three-stage method comprises, in addition to a stem cell mobilizing agent and Tpo, each of LMWH, Flt-3L, SCF, IL-6, IL-7, G-CSF, and GM-CSF. In certain aspects, the first medium used in the three-stage method comprises, in addition to a stem cell mobilizing agent and Tpo, each of Flt-3L, SCF, IL-6, IL-7, G-CSF, and GM-CSF. In a specific aspect, the first medium lacks added LMWH. In a specific aspect, the first medium lacks added desulphated glycosaminoglycans. In a specific aspect, the first medium lacks LMWH. In a specific aspect, the first medium lacks desulphated glycosaminoglycans. In certain aspects, said Tpo is present in the first medium at a concentration of from 1 ng/mL to 100 ng/mL, from 1 ng/mL to 50 ng/mL, from 20 ng/mL to 30 ng/mL, or about 25 ng/mL. In certain aspects, in the first medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the LMWH is present at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the LMWH is present at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the first medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the first medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said first medium is not GBGM®.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, Flt-3, SCF, IL-6, IL-7, G-CSF, and GM-CSF. In certain aspects, the second medium used in the three-stage method comprises, in addition to a stem cell mobilizing agent and IL-15, each of LMWH, Flt-3, SCF, IL-6, IL-7, G-CSF, and GM-CSF. In certain aspects, the second medium used in the three-stage method comprises, in addition to a stem cell mobilizing agent and IL-15, each of Flt-3, SCF, IL-6, IL-7, G-CSF, and GM-CSF. In a specific aspect, the second medium lacks added LMWH. In a specific aspect, the second medium lacks added desulphated glycosaminoglycans. In a specific aspect, the second medium lacks LMWH. In a specific aspect, the second medium lacks desulphated glycosaminoglycans. In certain aspects, said IL-15 is present in said second medium at a concentration of from 1 ng/mL to 50 ng/mL, from 10 ng/mL to 30 ng/mL, or about 20 ng/mL. In certain aspects, in said second medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said second medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the second medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said second medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the second medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said second medium is not GBGM®.

In certain embodiments, the third medium used in the three-stage method comprises medium comprising In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/mL. In certain aspects, said IL-2 is present in said third medium at a concentration of from 100 U/mL to 10,000 U/mL and said IL-15 is present in said third medium at a concentration of from 1 ng/mL to 50 ng/mL. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/mL. In certain aspects, in said third medium, the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said third medium, the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in said third medium, the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL. In certain embodiments, said third medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the third medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said third medium is not GBGM®.

Generally, the particularly recited medium components do not refer to possible constituents in an undefined component of said medium. For example, said Tpo, IL-2, and IL-15 are not comprised within an undefined component of the first medium, second medium or third medium, e.g., said Tpo, IL-2, and IL-15 are not comprised within serum. Further, said LMWH, Flt-3, SCF, IL-6, IL-7, G-CSF, and/or GM-CSF are not comprised within an undefined component of the first medium, second medium or third medium, e.g., said LMWH, Flt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB.

In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said second medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In a specific embodiment, in the three-stage methods described herein, said hematopoietic stem or progenitor cells are cultured in said first medium for 7-13 days to produce a first population of cells, before said culturing in said second medium; said first population of cells are cultured in said second medium for 2-6 days to produce a second population of cells before said culturing in said third medium; and said second population of cells are cultured in said third medium for 10-30 days, i.e., the cells are cultured a total of 19-49 days.

In a specific embodiment, in the three-stage methods described herein, in the three-stage methods described herein, said hematopoietic stem or progenitor cells are cultured in said first medium for 8-12 days to produce a first population of cells, before said culturing in said second medium; said first population of cells are cultured in said second medium for 3-5 days to produce a second population of cells before said culturing in said third medium; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells, before said culturing in said second medium; said first population of cells are cultured in said second medium for about 4 days to produce a second population of cells before said culturing in said third medium; and said second population of cells are cultured in said third medium for about 21 days, i.e., the cells are cultured a total of about 35 days.

In certain aspects, the three-stage method disclosed herein produces at least 5000-fold more natural killer cells as compared to the number of hematopoietic stem cells initially inoculated into said first medium. In certain aspects, said three-stage method produces at least 10,000-fold more natural killer cells as compared to the number of hematopoietic stem cells initially inoculated into said first medium. In certain aspects, said three-stage method produces at least 50,000-fold more natural killer cells as compared to the number of hematopoietic stem cells initially inoculated into said first medium. In certain aspects, said three-stage method produces at least 75,000-fold more natural killer cells as compared to the number of hematopoietic stem cells initially inoculated into said first medium. In certain aspects, the viability of said natural killer cells is determined by 7-aminoactinomycin D (7AAD) staining. In certain aspects, the viability of said natural killer cells is determined by annexin-V staining. In specific aspects, the viability of said natural killer cells is determined by both 7-AAD staining and annexin-V staining. In certain aspects, the viability of said natural killer cells is determined by trypan blue staining.

In certain aspects, the three-stage method produces natural killer cells that comprise at least 20% CD56+CD3− natural killer cells. In certain aspects, the three-stage method produces natural killer cells that comprise at least 40% CD56+CD3− natural killer cells. In certain aspects, the three-stage method produces natural killer cells that comprise at least 60% CD56+CD3− natural killer cells. In certain aspects, the three-stage method produces natural killer cells that comprise at least 70% CD56+CD3− natural killer cells. In certain aspects, the three-stage method produces natural killer cells that comprise at least 80% CD56+CD3− natural killer cells.

In certain aspects, the three-stage method produces natural killer cells that exhibit at least 20% cytotoxicity against K562 cells when said natural killer cells and said K562 cells are co-cultured in vitro at a ratio of 10:1. In certain aspects, the three-stage method produces natural killer cells that exhibit at least 35% cytotoxicity against the K562 cells when said natural killer cells and said K562 cells are co-cultured in vitro at a ratio of 10:1. In certain aspects, the three-stage method produces natural killer cells that exhibit at least 45% cytotoxicity against the K562 cells when said natural killer cells and said K562 cells are co-cultured in vitro at a ratio of 10:1. In certain aspects, the three-stage method produces natural killer cells that exhibit at least 60% cytotoxicity against the K562 cells when said natural killer cells and said K562 cells are co-cultured in vitro at a ratio of 10:1. In certain aspects, the three-stage method produces natural killer cells that exhibit at least 75% cytotoxicity against the K562 cells when said natural killer cells and said K562 cells are co-cultured in vitro at a ratio of 10:1.

In certain aspects, after said third culturing step, said third population of cells, e.g., said population of natural killer cells, is cryopreserved.

In certain aspects, provided herein are populations of cells comprising natural killer cells, i.e., natural killer cells produced by a three-stage method described herein. Accordingly, provided herein is an isolated natural killer cell population produced by a three-stage method described herein. In a specific embodiment, said natural killer cell population comprises at least 20% CD56+CD3− natural killer cells. In a specific embodiment, said natural killer cell population comprises at least 40% CD56+CD3− natural killer cells. In a specific embodiment, said natural killer cell population comprises at least 60% CD56+CD3− natural killer cells. In a specific embodiment, said natural killer cell population comprises at least 80% CD56+CD3− natural killer cells. In a specific embodiment, said natural killer cell population comprises at least 60% CD16− cells. In a specific embodiment, said natural killer cell population comprises at least 80% CD16− cells. In a specific embodiment, said natural killer cell population comprises at least 20% CD94+ cells. In a specific embodiment, said natural killer cell population comprises at least 40% CD94+ cells.

### 5.6.5. Chemistry Definitions

To facilitate understanding of the disclosure of stem cell mobilizing factors set forth herein, a number of terms are defined below.

Generally, the nomenclature used herein and the laboratory procedures in biology, cellular biology, biochemistry, organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or

“approximately” means within 50%, 20%, 15%, 10%, 9%, 8%, 7%, 6%, 5%, 4%, 3%, 2%, 1%, 0.5%, or 0.05% of a given value or range.

The term “aryl hydrocarbon receptor” or “AHR” refers to a protein encoded by the AHR gene in humans, or a variant thereof (for example, see GenBank Accession Nos. P35869.2 and AAH70080.1).

The term “aryl hydrocarbon receptor antagonist”, “AHR antagonist”, “aryl hydrocarbon receptor inhibitor,” or “AHR inhibitor” refers to a compound that downregulates or reduces the activity of an aryl hydrocarbon receptor.

The term “alkyl” refers to a linear or branched saturated monovalent hydrocarbon radical, wherein the alkyl is optionally substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C1-20), 1 to 15 (C1-15), 1 to 10 (C1-10), or 1 to 6 (C1-6) carbon atoms, or branched saturated monovalent hydrocarbon radical of 3 to 20 (C3-20), 3 to 15 (C3-15), 3 to 10 (C3-10), or 3 to 6 (C3-6) carbon atoms. As used herein, linear C1-6 and branched C3-6 alkyl groups are also referred as“lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C1-6 alkyl refers to a linear saturated monovalent hydrocarbon radical of 1 to 6 carbon atoms or a branched saturated monovalent hydrocarbon radical of 3 to 6 carbon atoms.

The term “alkylene” refers to a linear or branched saturated divalent hydrocarbon radical, wherein the alkylene is optionally substituted with one or more substituents Q as described herein. For example, C1-6 alkylene refers to a linear saturated divalent hydrocarbon radical of 1 to 6 carbon atoms or a branched saturated divalent hydrocarbon radical of 3 to 6 carbon atoms. In certain embodiments, the alkylene is a linear saturated divalent hydrocarbon radical that has 1 to 20 (C1-20), 1 to 15 (C1-15), 1 to 10 (C1-10), or 1 to 6 (C1-6) carbon atoms, or branched saturated divalent hydrocarbon radical of 3 to 20 (C3-20), 3 to 15 (C3-15), 3 to 10 (C3-10), or 3 to 6 (C3-6) carbon atoms. As used herein, linear C1-6 and branched C3-6 alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms).

The term “alkenyl” refers to a linear or branched monovalent hydrocarbon radical, which contains one or more, in one embodiment, one, two, three, four, or five, in another embodiment, one, carbon-carbon double bond(s). The alkenyl is optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C2-6 alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C2-20), 2 to 15 (C2-15), 2 to 10 (C2-10), or 2 to 6 (C2-6) carbon atoms, or a branched monovalent hydrocarbon radical of 3 to 20 (C3-20), 3 to 15 (C3-15), 3 to 10 (C3-10), or 3 to 6 (C3-6) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one to five, in another embodiment, one, carbon-carbon double bond(s). The alkenylene is optionally substituted with one or more substituents Q as described herein. The term “alkenylene” embraces radicals having a “cis” or “trans” configuration or a mixture thereof, or alternatively, a “Z” or “E” configuration or a mixture thereof, as appreciated by those of ordinary skill in the art. For example, C2-6 alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C2-20), 2 to 15 (C2-15), 2 to 10 (C2-10), or 2 to 6 (C2-6) carbon atoms, or a branched divalent hydrocarbon radical of 3 to 20 (C3-20), 3 to 15 (C3-15), 3 to 10 (C3-10), or 3 to 6 (C3-6) carbon atoms. Examples of alkenylene groups include, but are not limited to, ethenylene, allylene, propenylene, butenylene, and 4-methylbutenylene.

The term “alkynyl” refers to a linear or branched monovalent hydrocarbon radical, which contains one or more, in one embodiment, one, two, three, four, or five, in another embodiment, one, carbon-carbon triple bond(s). The alkynyl is optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C2-20), 2 to 15 (C2-15), 2 to 10 (C2-10), or 2 to 6 (C2-6) carbon atoms, or a branched monovalent hydrocarbon radical of 3 to 20 (C3-20), 3 to 15 (C3-15), 3 to 10 (C3-10), or 3 to 6 (C3-6) carbon atoms. Examples of alkynyl groups include, but are not limited to, ethynyl (—C≡CH) and propargyl (—CH2C≡CH). For example, C2-6 alkynyl refers to a linear unsaturated monovalent hydrocarbon radical of 2 to 6 carbon atoms or a branched unsaturated monovalent hydrocarbon radical of 3 to 6 carbon atoms.

The term “alkynylene” refers to a linear or branched divalent hydrocarbon radical, which contains one or more, in one embodiment, one to five, in another embodiment, one, carbon-carbon triple bond(s). The alkynylene is optionally substituted with one or more substituents Q as described herein. For example, C2-6 alkynylene refers to a linear unsaturated divalent hydrocarbon radical of 2 to 6 carbon atoms or a branched unsaturated divalent hydrocarbon radical of 3 to 6 carbon atoms. In certain embodiments, the alkynylene is a linear divalent hydrocarbon radical of 2 to 20 (C2-20), 2 to 15 (C2-15), 2 to 10 (C2-10), or 2 to 6 (C2-6) carbon atoms, or a branched divalent hydrocarbon radical of 3 to 20 (C3-20), 3 to 15 (C3-15), 3 to 10 (C3-10), or 3 to 6 (C3-6) carbon atoms. Examples of alkynylene groups include, but are not limited to, ethynylene, propynylene (including all isomeric forms, e.g., 1-propynylene and propargylene), butynylene (including all isomeric forms, e.g., 1-butyn-1-ylene and 2-butyn-1-ylene), pentynylene (including all isomeric forms, e.g., 1-pentyn-1-ylene and 1-methyl-2-butyn-1-ylene), and hexynylene (including all isomeric forms, e.g., 1-hexyn-1-ylene).

The term “cycloalkyl” refers to a cyclic saturated or non-aromatic unsaturated, bridged or non-bridged monovalent hydrocarbon radical, which is optionally substituted with one or more substituents Q as described herein. In certain embodiments, the cycloalkyl is a cyclic saturated bridged or non-bridged monovalent hydrocarbon radical. In certain embodiments, the cycloalkyl has from 3 to 20 (C3-20), from 3 to 15 (C3-15), from 3 to 10 (C3-10), or from 3 to 7 (C3-7) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “cycloalkylene” refers to a cyclic divalent hydrocarbon radical, which is optionally substituted with one or more substituents Q as described herein. In one embodiment, cycloalkyl groups is saturated or unsaturated but non-aromatic, and/or bridged, and/or non-bridged, and/or fused bicyclic groups. In certain embodiments, the cycloalkylene has from 3 to 20 (C3-20), from 3 to 15 (C3-15), from 3 to 10 (C3-10), or from 3 to 7 (C3-7) carbon atoms. Examples of cycloalkylene groups include, but are not limited to, cyclopropylene (e.g., 1,1-cyclopropylene and 1,2-cyclopropylene), cyclobutylene (e.g., 1,1-cyclobutylene, 1,2-cyclobutylene, or 1,3-cyclobutylene), cyclopentylene (e.g., 1,1-cyclopentylene, 1,2-cyclopentylene, or 1,3-cyclopentylene), cyclohexylene (e.g., 1,1-cyclohexylene, 1,2-cyclohexylene, 1,3-cyclohexylene, or 1,4-cyclohexylene), cycloheptylene (e.g., 1,1-cycloheptylene, 1,2-cycloheptylene, 1,3-cycloheptylene, or 1,4-cycloheptylene), decalinylene, and adamantylene.

The term “aryl” refers to a monocyclic aromatic carbocyclic group and/or multicyclic monovalent aromatic carbocyclic group that contain at least one aromatic ydrocarbon ring. In certain embodiments, the aryl has from 6 to 20 (C6-20), from 6 to 15 (C6-15), or from 6 to 10 (C6-10) ring atoms. Examples of aryl groups include, but are not limited to, phenyl, naphthyl, fluorenyl, azulenyl, anthryl, phenanthryl, pyrenyl, biphenyl, and terphenyl. In certain embodiments, the term “aryl” refers to a bicyclic or tricyclic carbon ring, where one of the rings is aromatic and the others of which can be saturated, partially unsaturated, or aromatic, for example, dihydronaphthyl, indenyl, indanyl, or tetrahydronaphthyl (tetralinyl). The aryl is optionally substituted with one or more substituents Q as described herein.

The term “arylene” refers to a divalent monocyclic aromatic group and/or divalent polycyclic aromatic group that contain at least one aromatic carbon ring. In certain embodiments, the arylene has from 6 to 20 (C6-20), from 6 to 15 (C6-15), or from 6 to 10 (C6-10) ring atoms. Examples of arylene groups include, but are not limited to, phenylene, naphthylene, fluorenylene, azulenylene, anthrylene, phenanthrylene, pyrenylene, biphenylene, and terphenylene. Arylene also refers to bicyclic or tricyclic carbon rings, where one of the rings is aromatic and the others of which can be saturated, partially unsaturated, or aromatic, for example, dihydronaphthylene, indenylene, indanylene, or tetrahydronaphthylene (tetralinylene). The arylene is optionally substituted with one or more substituents Q as described herein.

The term “aralkyl” or“arylalkyl” refers to a monovalent alkyl group substituted with one or more aryl groups. In certain embodiments, the aralkyl has from 7 to 30 (C7-30), from 7 to 20 (C7-20), or from 7 to 16 (C7-16) carbon atoms. Examples of aralkyl groups include, but are not limited to, benzyl, 1-phenylethyl, 2-phenylethyl, and 3-phenylpropyl. The aralkyl is optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms, each of which is independently selected from O, S, N, and P, in the ring. For clarity, the terms “aryl” and “heteroaryl” as used herein are mutually exclusive, i.e., “aryl” groups do not include “heteroaryl” groups, and vice versa. A heteroaryl group is bonded to the rest of a molecule through its aromatic ring. Each ring of a heteroaryl group can contain one or two O atoms, one or two S atoms, one to four N atoms, and/or one or two P atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. The heteroaryl is optionally substituted with one or more substituents Q as described herein.

The term “heteroarylene” refers to a divalent monocyclic aromatic group or divalent polycyclic aromatic group that contain at least one aromatic ring, wherein at least one aromatic ring contains one or more heteroatoms independently selected from O, S, and N in the ring. For clarity, the terms “arylene” and “heteroarylene” as used herein are mutually exclusive, i.e., “arylene” groups do not include “heteroarylene” groups, and vice versa. A heteroarylene group is bonded to the rest of a molecule through its aromatic ring. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furanylene, imidazolylene, isothiazolylene, isoxazolylene, oxadiazolylene, oxadiazolylene, oxazolylene, pyrazinylene, pyrazolylene, pyridazinylene, pyridylene, pyrimidinylene, pyrrolylene, thiadiazolylene, thiazolylene, thienylene, tetrazolylene, triazinylene, and triazolylene. Examples of bicyclic heteroarylene groups include, but are not limited to, benzofuranylene, benzimidazolylene, benzoisoxazolylene, benzopyranylene, benzothiadiazolylene, benzothiazolylene, benzothienylene, benzotriazolylene, benzoxazolylene, furopyridylene, imidazopyridinylene, imidazothiazolylene, indolizinylene, indolylene, indazolylene, isobenzofuranylene, isobenzothienylene, isoindolylene, isoquinolinylene, isothiazolylene, naphthyridinylene, oxazolopyridinylene, phthalazinylene, pteridinylene, purinylene, pyridopyridylene, pyrrolopyridylene, quinolinylene, quinoxalinylene, quinazolinylene, thiadiazolopyrimidylene, and thienopyridylene. Examples of tricyclic heteroarylene groups include, but are not limited to, acridinylene, benzindolylene, carbazolylene, dibenzofuranylene, perimidinylene, phenanthrolinylene, phenanthridinylene, phenarsazinylene, phenazinylene, phenothiazinylene, phenoxazinylene, and xanthenylene. The heteroarylene is optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms, each of which is independently selected from O, S, N, and P; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. A heterocyclyl group is bonded to the rest of a molecule through its non-aromatic ring. In certain embodiments, the heterocyclyl is a monocyclic, bicyclic, tricyclic, or tetracyclic ring system, which can be spiro, fused, or bridged, and in which nitrogen or sulfur atoms can be optionally oxidized, nitrogen atoms can be optionally quaternized, and some rings can be partially or fully saturated, or aromatic. The heterocyclyl can be attached to the main structure at any heteroatom or carbon atom which results in the creation of a stable compound. Examples of heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. The heterocyclyl is optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which can be fused or bridged, and in which nitrogen or sulfur atoms can be optionally oxidized, nitrogen atoms can be optionally quaternized, and some rings can be partially or fully saturated, or aromatic. The heterocyclylene can be attached to the main structure at any heteroatom or carbon atom which results in the creation of a stable compound. Examples of such heterocyclylene groups include, but are not limited to, azepinylene, benzodioxanylene, benzodioxolylene, benzofuranonylene, benzopyranonylene, benzopyranylene, benzotetrahydrofuranylene, benzotetrahydrothienylene, benzothiopyranylene, benzoxazinylene, O-carbolinylene, chromanylene, chromonylene, cinnolinylene, coumarinylene, decahydroisoquinolinylene, dihydrobenzisothiazinylene, dihydrobenzisoxazinylene, dihydrofurylene, dihydroisoindolylene, dihydropyranylene, dihydropyrazolylene, dihydropyrazinylene, dihydropyridinylene, dihydropyrimidinylene, dihydropyrrolylene, dioxolanylene, 1,4-dithianylene, furanonylene, imidazolidinylene, imidazolinylene, indolinylene, isobenzotetrahydrofuranylene, isobenzotetrahydrothienylene, isochromanylene, isocoumarinylene, isoindolinylene, isothiazolidinylene, isoxazolidinylene, morpholinylene, octahydroindolylene, octahydroisoindolylene, oxazolidinonylene, oxazolidinylene, oxiranylene, piperazinylene, piperidinylene, 4-piperidonylene, pyrazolidinylene, pyrazolinylene, pyrrolidinylene, pyrrolinylene, quinuclidinylene, tetrahydrofurylene, tetrahydroisoquinolinylene, tetrahydropyranylene, tetrahydrothienylene, thiamorpholinylene, thiazolidinylene, tetrahydroquinolinylene, and 1,3,5-trithianylene. The heterocyclylene is optionally substituted with one or more substituents Q as described herein.

The term “halogen”, “halide” or “halo” refers to fluorine, chlorine, bromine, and/or iodine.

The term “haloalkyl” refers to an alkyl group substituted with one or more, in one embodiment, one, two, or three, halo groups, where the alkyl is as defined herein. The haloalkyl is optionally substituted with one or more substituents Q as described herein.

The term “alkoxy” refers to —O-alkyl, where the alkyl is as defined herein.

The term “haloalkoxy” refers to —O-haloalkyl, where the haloalkyl is as defined herein.

The term “optionally substituted” is intended to mean that a group or substituent, such as an alkyl, alkylene, alkenyl, alkenylene, alkynyl, alkynylene, cycloalkyl, cycloalkylene, aryl, arylene, aralkyl (e.g., benzyl), heteroaryl, heteroarylene, heterocyclyl, and heterocyclylene group, may be substituted with one or more substituents Q, each of which is independently selected from, e.g., (a) oxo (═O), cyano (—CN), halo, and nitro (—NO2); (b) C1-6 alkyl, C2-6 alkenyl, C2-6 alkynyl, C3-10 cycloalkyl, C6-14 aryl, C7-15 aralkyl, heteroaryl, and heterocyclyl, each of which is further optionally substituted with one or more, in one embodiment, one, two, three, four, or five, substituents Qa; and (c) —C(O)Ra, —C(O)ORa, —C(O)NRbRc, —C(NRa)NRbRc, —ORa, —OC(O)Ra, —OC(O)ORa, —OC(O)NRbRc, —OC(═NRa)NRbRc, —OS(O)Ra, —OS(O)2Ra, —OS(O)NRbRc, —OS(O)2NRbRc, —NRbRc, —NRaC(O)Rd, —NRaC(O)ORd, —NRaC(O)NRbRc, —NRaC(═NRd)NRbRc, —NRaS(O)Rd, —NRaS(O)2Rd, —NRaS(O)NRbRc, —NRaS(O)2NRbRc, —P(O)RaRd, —P(O)(ORa)Rd, —P(O)(ORa)(ORd), —SRa, —S(O)Ra, —S(O)2Ra, —S(O)NRbRc, and —S(O)2NRbRc, wherein each Ra, Rb, Rc, and Rd is independently (i) hydrogen; (ii) C1-6 alkyl, C2-6 alkenyl, C2-6 alkynyl, C3-10 cycloalkyl, C6-14 aryl, C7-15 aralkyl, heteroaryl, or heterocyclyl, each of which is optionally substituted with one or more, in one embodiment, one, two, three, or four, substituents Qa; or (iii) Rb and Rc together with the N atom to which they are attached form heteroaryl or heterocyclyl, each of which is optionally substituted with one or more, in one embodiment, one, two, three, or four, substituents Qa. As used herein, all groups described herein that can be substituted are“optionally substituted,” unless otherwise specified.

In one embodiment, each substituent Qa is independently selected from the group consisting of (a) oxo, cyano, halo, and nitro; and (b) C1-6 alkyl, C2-6 alkenyl, C2-6 alkynyl, C3-10 cycloalkyl, C6-14 aryl, C7-15 aralkyl, heteroaryl, and heterocyclyl; and (c) —C(O)Re, —C(O)ORe, —C(O)NRfRg, —C(NRe)NRfRg, —ORe, —OC(O)Re, —OC(O)ORe, —OC(O)NRfRg, —OC(═NRe)NRfRg, —OS(O)Re, —OS(O)2Re, —OS(O)NRfRg, —OS(O)2NRfRg, —NRfRg, —NReC(O)Rh, —NReC(O)ORh, —NReC(O)NRfRg, —NReC(═NRh)NRfRg, —NReS(O)Rh, —NReS(O)2Rh, —NReS(O)NRfRg, —NReS(O)2NRfRg, —P(O)ReRh, —P(O)(ORe)Rh, —P(O)(ORe)(ORh), —SRe, —S(O)Re, —S(O)2Re, —S(O)NRfRg, and —S(O)2NRfRg; wherein each Re, Rf, Rg, and Rh is independently (i) hydrogen, C1-6 alkyl, C2-6 alkenyl, C2-6 alkynyl, C3-10 cycloalkyl, C6-14 aryl, C7-15 aralkyl, heteroaryl, or heterocyclyl; or (ii) Rf and Rg together with the N atom to which they are attached form heteroaryl or heterocyclyl.

In certain embodiments, “optically active” and“enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the two enantiomers in question.

In describing an optically active compound, the prefixes R and S are used to denote the absolute configuration of the optically active compound about its chiral center(s). The (+) and (−) are used to denote the optical rotation of an optically active compound, that is, the direction in which a plane of polarized light is rotated by the optically active compound. The (−) prefix indicates that an optically active compound is levorotatory, that is, the compound rotates the plane of polarized light to the left or counterclockwise. The (+) prefix indicates that an optically active compound is dextrorotatory, that is, the compound rotates the plane of polarized light to the right or clockwise. However, the sign of optical rotation, (+) and (−), is not related to the absolute configuration of a compound, R and S.

The term “isotopic variant” refers to a compound that contains an unnatural proportion of an isotope at one or more of the atoms that constitute such a compound. In certain embodiments, an“isotopic variant” of a compound contains unnatural proportions of one or more isotopes, including, but not limited to, hydrogen (1H), deuterium (2H), tritium (3H), carbon-11 (11C), carbon-12 (12C), carbon-13 (13C), carbon-14 (14C), nitrogen-13 (13N), nitrogen-14 (14N), nitrogen-15 (15N), oxygen-14 (14O), oxygen-15 (15O), oxygen-16 (16O), oxygen-17 (17O), oxygen-18 (18O), fluorine-17 (17F), fluorine-18 (18F), phosphorus-31 (31P), phosphorus-32 (32P), phosphorus-33 (33P), sulfur-32 (32S), sulfur-33 (33S), sulfur-34 (34S), sulfur-35 (35S), sulfur-36 (36S), chlorine-35 (35Cl), chlorine-36 (36Cl), chlorine-37 (37Cl), bromine-79 (79Br), bromine-81 (81Br), iodine-123 (123I), iodine-125 (125I), iodine-127 (127I), iodine-129 (129I), and iodine-131 (131I). In certain embodiments, an “isotopic variant” of a compound is in a stable form, that is, non-radioactive. In certain embodiments, an“isotopic variant” of a compound contains unnatural proportions of one or more isotopes, including, but not limited to, hydrogen (1H), deuterium (2H), carbon-12 (12C), carbon-13 (13C), nitrogen-14 (14N), nitrogen-15 (15N), oxygen-16 (16O), oxygen-17 (17O), oxygen-18 (18O), fluorine-17 (17F), phosphorus-31 (31P), sulfur-32 (32S), sulfur-33 (33S), sulfur-34 (34S), sulfur-36 (36S), chlorine-35 (35Cl), chlorine-37 (37Cl), bromine-79 (79Br), bromine-81 (81Br), and iodine-127 (127I). In certain embodiments, an “isotopic variant” of a compound is in an unstable form, that is, radioactive. In certain embodiments, an“isotopic variant” of a compound contains unnatural proportions of one or more isotopes, including, but not limited to, tritium (3H), carbon-11 (11C), carbon-14 (14C), nitrogen-13 (13N), oxygen-14 (14O), oxygen-15 (15O), fluorine-18 (18F), phosphorus-32 (32P), phosphorus-33 (33P), sulfur-35 (35S), chlorine-36 (36Cl), iodine-123 (123I), iodine-125 (125I), iodine-129 (129I), and iodine-131 (131I). It will be understood that, in a compound as provided herein, any hydrogen can be 2H, for example, or any carbon can be 13C, for example, or any nitrogen can be 15N, for example, or any oxygen can be 180, for example, where feasible according to the judgment of one of skill. In certain embodiments, an “isotopic variant” of a compound contains unnatural proportions of deuterium (D).

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a mixture of two or more diastereomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, or an isotopic variant of the compound referenced therein.”

### 5.6.6. Stem Cell Mobilizing Agents

In one embodiment, the stem cell mobilizing compound is an aryl hydrocarbon receptor inhibitor, e.g., an aryl hydrocarbon receptor antagonist.

In another embodiment, the stem cell mobilizing compound is a 5,6-fused heteroaryl compound, including, but not limited to, those described in U.S. Pat. App. Pub. Nos. 2010/0183564, 2014/0023626, and 2014/0114070, the disclosure of each of which is incorporated herein by reference in its entirety.

In yet another embodiment, the stem cell mobilizing compound is a compound of Formula I:

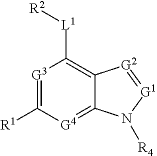

or an enantiomer, a mixture of enantiomers, a mixture of two or more diastereomers, or an isotopic variant thereof, or a pharmaceutically acceptable salt, solvate, hydrate, or prodrug thereof; wherein:

### G1 is N and CR3;

G2, G3, and G4 are each independently CH and N; with the proviso that at least one of G3 and G4 is N, and at least one of G1 and G2 is not N;
L1 is —NR1a-, —NR1a(CH2)1-3-, —NR1aCH(C(O)OCH3)CH2-, —NR1a(CH2)2NR1c-, —NR1a(CH2)2S—, —NR1aCH2CH(CH3)CH2-, —NR1aCH2CH(OH)—, or —NR1aCH(CH3)CH2-;
R1 is (i) hydrogen; or (ii) phenyl, furanyl, pyrrolyl, imidazolyl, pyrazolyl, thienyl, thiazolyl, pyridinyl, pyrimidinyl, pyrrolidinyl, pyrazinyl, pyridazinyl, benzoimidazolyl, isoquinolinyl, imidazopyridinyl, or benzothienyl, each of which is optionally substituted by one, two, or three substituents, where each substituent is independently cyano, halo, C1-4 alkyl, C1-4 alkoxy, C1-4 haloalkyl, C1-4 haloalkoxy, hydroxyl, amino, —C(O)R1a, —C(O)OR1a, —C(O)NR1aR1b, —SR1a, —S(O)R1a, or —S(O)2R1a;
R2 is (i) —NR1aC(O)R1c, —NR1cC(O)NR1aR1b, or —S(O)2NR1aR1b; or (ii) phenyl, pyrrolopyridin-3-yl, indolyl, thienyl, pyridinyl, 1,2,4-triazolyl, 2-oxoimidazolidinyl, pyrazolyl, 2-oxo-2,3-dihydro-1H-benzoimidazolyl, or indazolyl, each of which is optionally substituted with one, two, or three substituents, where each substituent is independently hydroxyl, halo, methyl, methoxy, amino, —O(CH2)1-3NR1aR1b, —OS(O)2NR1aR1b, —NR1aS(O)2R1b, or —S(O)2NR1aR1b;
R3 is hydrogen, C1-4 alkyl, or biphenyl; with the proviso that at least one of R1 and R3 is not hydrogen;
R4 is C1-10 alkyl, prop-1-en-2-yl, cyclohexyl, cyclopropyl, 2-(2-oxopyrrolidin-1-yl)ethyl, oxetan-3-yl, benzhydryl, tetrahydro-2H-pyran-3-yl, tetrahydro-2H-pyran-4-yl, phenyl, tetrahydrofuran-3-yl, benzyl, (4-pentylphenyl)(phenyl)methyl, or 1-(1-(2-oxo-6,9,12-trioxa-3-azatetradecan-14-yl)-1H-1,2,3-triazol-4-yl)ethyl, each of which is optionally substituted with one, two, or three substituents, where each substituent is independently hydroxyl, C1-4 alkyl, or C1-4 haloalkyl; and
each R1a, R1b, and R1c is independently hydrogen or C1-4 alkyl; or R1a and R1b together with the N atom to which they are attached form heterocyclyl.

In one embodiment, in Formula I, G1 is CR3, in one embodiment, CH; G2, G3, and G4 are each N; and R1, R2, R3, R4, and L1 are each as defined herein.

In another embodiment, in Formula I, G1, G3, and G4 are each N; G2 is CH; and R1, R2, R4, and L1 are each as defined herein.

In yet another embodiment, in Formula I, G1 is CR3, in one embodiment, CH; G2 and G3 are each N; G4 is CH; and R1, R2, R3, R4, and L1 are each as defined herein.

In yet another embodiment, in Formula I, G1 is CR3, in one embodiment, CH; G2 and

G4 are each N; G3 is CH; and R1, R2, R3, R4, and L1 are each as defined herein. In yet another embodiment, in Formula I, G1 is CR3, in one embodiment, CH; G2 is CH; G3 and G4 are each N; and R1, R2, R3, R4, and L1 are each as defined herein.

In still embodiment, in Formula I,

### G1 is CH;

G2, G3, and G4 are each N;
R1 is benzothienyl, optionally substituted by one, two, or three substituents, each of which is independently cyano, halo, C1-4 alkyl, C1-4 alkoxy, C1-4 haloalkyl, C1-4 haloalkoxy, hydroxyl, amino, —C(O)R1a, —C(O)OR1a, —C(O)NR1aR1b, —SR1a, —S(O)R1a, or —S(O)2R1a;
R2 is phenyl, optionally substituted with one, two, or three substituents, each of which is independently hydroxyl, halo, methyl, methoxy, amino, —O(CH2)1-3NR1aR1b, —OS(O)2NR1aR1b, —NR1aS(O)2R1b, or —S(O)2NR1aR1b;
R4 is C1-10 alkyl, optionally substituted with one, two, or three substituents, each of which is independently hydroxyl, C1-4 alkyl, or C1-4 haloalkyl;

### L1 is —NR1a(CH2)2-; and

R1a and R1b are each as defined herein.

In yet another embodiment, the stem cell mobilizing compound is a compound of Formula II:

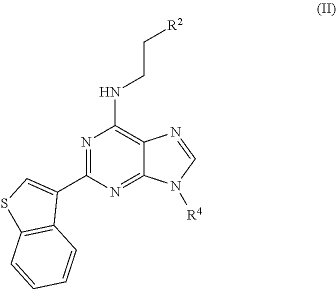

or an enantiomer, a mixture of enantiomers, a mixture of two or more diastereomers, or an isotopic variant thereof; or a pharmaceutically acceptable salt, solvate, hydrate, or prodrug thereof; wherein R2 and R4 are each as defined herein.

In yet another embodiment, the stem cell mobilizing compound is a compound of Formula III:

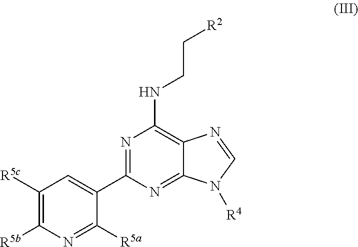

or an enantiomer, a mixture of enantiomers, a mixture of two or more diastereomers, or an isotopic variant thereof; or a pharmaceutically acceptable salt, solvate, hydrate, or prodrug thereof; wherein R2 and R4 are each as defined herein; and R5a, R5b, and R5c are each independently hydrogen, cyano, methyl, halo, trifluoromethyl, or —SO2CH3.

In yet another embodiment, the stem cell mobilizing compound is 4-(2-(2-(benzo[b]thien-3-yl)-9-isopropyl-9H-purin-6-ylamino)ethyl)phenol. In certain embodiments, the stem cell mobilizing compound is StemRe enin-1 SR-1 having the structure of:

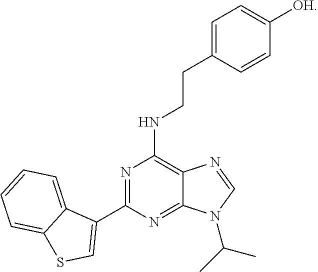 [SMILES: CC(C)n1cnc2c(NCCc3ccc(O)cc3)nc(-c3csc4ccccc34)nc21]

In yet another embodiment, the stem cell mobilizing compound is 1-methyl-N-(2-methyl-4-(2-(2-methylphenyl)diazenyl)phenyl)-1H-pyrazole-5-carboxamide. In certain embodiments, the stem cell mobilizin compound is CH223191, which has the structure of:

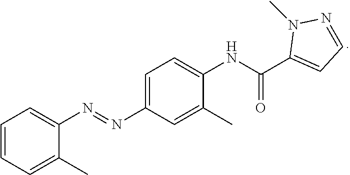 [SMILES: Cc1ccccc1/N=N/c1ccc(NC(=O)c2ccnn2C)c(C)c1]

In yet another embodiment, the stem cell mobilizing compound is a pyrimido(4,5-b)indole.

In yet another embodiment, the stem cell mobilizing compound is a compound of Formula IV:

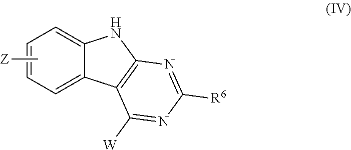

or an enantiomer, a mixture of enantiomers, a mixture of two or more diastereomers, or an isotopic variant thereof, or a pharmaceutically acceptable salt, solvate, hydrate, or prodrug thereof, wherein:

Z is cyano, C1-6 alkyl, C2-6 alkenyl, C2-6 alkynyl, C3-10 cycloalkyl, C6-14 aryl, C7-15 aralkyl, benzyl, heteroaryl, heterocyclyl, -L-C6-14 aryl, -L-heteroaryl, -L-heterocyclyl, —C(O)R1a, —C(O)OR1a, —C(O)NHR1a, —C(O)N(R1a)R1b, —P(O)(OR1a)(OR1c), —SR1a, —S(O)R1a, —S(O)2R1a, —S(O)2NH2, —S(O)2NHR1a, or —S(O)2N(R1a)R1b; W is hydrogen, halo, cyano, C6-14 aryl, benzyl, heteroaryl, heterocyclyl, -L-C6-14 aryl, -L-heteroaryl, -L-heterocyclyl, -L-OH, -L-OR1a, -L-NH2, -L-NHR1a, -L-N(R1a)R1b, -L-SR1a, -L-S(O)R1a, -L-S(O)2R1a, -L-P(O)(OR1a)(OR1c), -L-(N(R1c)-L)n-N(R1a)R1b, -L-(N(R1c)-L)n-C6-14 aryl, -L-(N(R1c)-L)n-heteroaryl, -L-(N(R1c)-L)n-heterocyclyl, —O-L-N(R1a)R1b, —O-L-C6-14 aryl, —O-L-heteroaryl, —O-L-heterocyclyl, —O-L-(N(R1c)-L)n-N(R1a)R1b, —O-L-(N(R1c)-L)n-C6-14 aryl, —O-L-(N(R1c)-L)n-heteroaryl, —O-L-(N(R1c)-L)n-heterocyclyl, —S-L-N(R1a)R1b, —S-L-C6-14 aryl, —S-L-heteroaryl, —S-L-heterocyclyl, —S-L-(N(R1c)-L)n-N(R1a)R1b, —S-L-(N(R1c)-L)n-C6-14 aryl, —S-L-(N(R1c)-L)n-heteroaryl, —S-L-(N(R1c)-L)n-heterocyclyl, —(N(R1c)-L)n-N(R1a)R1b, —(N(R1c)-L)n-C6-14 aryl, —(N(R1c)-L)n-heteroaryl, —(N(R1c)-L)n-heterocyclyl, —C(O)R1a, —C(O)OR1a, —C(O)NH2, —C(O)NHR1a, —C(O)N(R1a)R1b, —NHR1a, —N(R1a)R1b, —NHC(O)R1a, —NR1aC(O)R1c, —NHC(O)OR1a, —NR1aC(O)OR1c, —NHC(O)NH2, —NHC(O)NHR1a, —NHC(O)N(R1a)R1b, —NR1aC(O)NH2, —NR1cC(O)NHR1a, —NR1cC(O)N(R1a)R1b, —NHS(O)2R1a, —NR1cS(O)2R1a, —OR1a, —OC(O)R1a, —OC(O)OR1a, —OC(O)NH2, —OC(O)NHR1a, —OC(O)N(R1a)R1b, —O S(O)2R1a, —P(O)(OR1a)(OR1c), —SR1a, —S(O)R1a, —S(O)2R1a, —S(O)2NH2, —S(O)2NHR1a, —S(O)2N(R1a)R1b, or —S(O)20R1a; each L is independently C1-6 alkylene, C2-6 alkenylene, C2-6 alkynylene, C3-7 cycloalkylene, C6-14 arylene, heteroarylene, heterocyclylene, C1-6 alkylene-C3-7 cycloalkylene, or C1-6 alkylene-heterocyclylene;

R6 is hydrogen, C1-6 alkyl, C6-14 aryl, benzyl, heteroaryl, —C(O)R1a, —SR1a, —S(O)R1a, —S(O)2R1a, -L-C6-14 aryl, -L-heteroaryl, or -L-heterocyclyl;

each n is independently an integer of 1, 2, 3, 4, or 5; and
each R1a, R1b, and R1c is independently (i) hydrogen; (ii) C1-6 alkyl, C2-6 alkenyl, C2-6 alkynyl, C3-10 cycloalkyl, C6-14 aryl, C7-15 aralkyl, heteroaryl, or heterocyclyl; or (iii) R1a and R1b together with the N atom to which they are attached form heterocyclyl;
wherein each alkyl, alkylene, alkenyl, alkenylene, alkynyl, alkynylene, cycloalkyl, cycloalkylene, aryl, benzyl, arylene, heteroaryl, heteroarylene, heterocyclyl, and heterocyclylene is optionally substituted with one or more, in one embodiment, one, two, three, or four, substituents Q, wherein each substituent Q is independently selected from (a) oxo, cyano, halo, and nitro; (b) C1-6 alkyl, C2-6 alkenyl, C2-6 alkynyl, C3-10 cycloalkyl, C6-14 aryl, C7-15 aralkyl, heteroaryl, and heterocyclyl, each of which is further optionally substituted with one or more, in one embodiment, one, two, three, or four, substituents Qa; and (c) —C(O)Ra, —C(O)ORa, —C(O)NRbRc, —C(NRa)NRbRc, —ORa, —OC(O)Ra, —OC(O)ORa, —OC(O)NRbRc, —OC(═NRa)NRbRc, —OS(O)Ra, —OS(O)2Ra, —OS(O)NRbRc, —OS(O)2NRbRc, —NRbRc, —NRaC(O)Rd, —NRaC(O)ORd, —NRaC(O)NRbRc, —NRaC(═NRd)NRbRc, —NRaS(O)Rd, —NRaS(O)2Rd, —NRaS(O)NRbRc, —NRaS(O)2NRbRc, —SRa, —S(O)Ra, —S(O)2Ra, —S(O)NRbRc, and —S(O)2NRbRc, wherein each Ra, Rb, Rc, and Rd is independently (i) hydrogen; (ii) C1-6 alkyl, C2-6 alkenyl, C2-6 alkynyl, C3-10 cycloalkyl, C6-14 aryl, C7-15 aralkyl, heteroaryl, or heterocyclyl, each of which is further optionally substituted with one or more, in one embodiment, one, two, three, or four, substituents Qa; or (iii) Rb and Rc together with the N atom to which they are attached form heterocyclyl, which is further optionally substituted with one or more, in one embodiment, one, two, three, or four, substituents Qa;
wherein each Qa is independently selected from the group consisting of (a) oxo, cyano, halo, and nitro; (b) C1-6 alkyl, C2-6 alkenyl, C2-6 alkynyl, C3-10 cycloalkyl, C6-14 aryl, C7-15 aralkyl, heteroaryl, and heterocyclyl; and (c) —C(O)Re, —C(O)ORe, —C(O)NRfRg, —C(NRe)NRfRg, —ORe, —OC(O)Re, —OC(O)ORe, —OC(O)NRfRg, —OC(═NRe)NRfRg, —OS(O)Re, —OS(O)2Re, —OS(O)NRfRg, —OS(O)2NRfRg, —NRfRg, —NReC(O)Rh, —NReC(O)ORh, —NReC(O)NRfRg, —NReC(═NRh)NRfRg, —NReS(O)Rh, —NReS(O)2Rh, —NReS(O)NRfRg, —NReS(O)2NRfRg, —SRe, —S(O)Re, —S(O)2Re, —S(O)NRfRg, and —S(O)2NRfRg; wherein each Re, Rf, Rg, and Rh is independently (i) hydrogen; (ii) C1-6 alkyl, C2-6 alkenyl, C2-6 alkynyl, C3-10 cycloalkyl, C6-14 aryl, C7-15 aralkyl, heteroaryl, or heterocyclyl; or (iii) Rf and Rg together with the N atom to which they are attached form heterocyclyl.

In yet another embodiment, the stem cell mobilizing compound is a compound of Formula V:

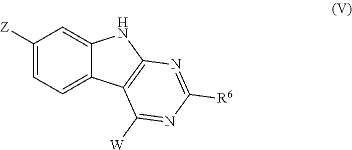

or an enantiomer, a mixture of enantiomers, a mixture of two or more diastereomers, or an isotopic variant thereof; or a pharmaceutically acceptable salt, solvate, hydrate, or prodrug thereof; wherein R6, W, and Z are each as defined herein.

In one embodiment, in Formula IV or V,

Z is cyano, heteroaryl, or —C(O)OR1a;
W is heterocyclyl, -L-heterocyclyl, —O-L-heterocyclyl, —(N(R1c)-L)n-N(R1a)R1b, —(N(R1c)-L)n-heterocyclyl, —NHR1a, or —N(R1a)R1b;
each L is independently C1-6 alkylene or C3-7 cycloalkylene;
R6 is hydrogen, C1-6 alkyl, benzyl, —C(O)R1a, -L-C6-14 aryl, or -L-heteroaryl;
each n is independently an integer of 1; and
R1a, R1b, and R1c are each as defined herein;
wherein each alkyl, alkylene, cycloalkylene, aryl, benzyl, heteroaryl, and heterocyclyl is optionally substituted with one or more substituents Q as defined herein.

In another embodiment, in Formula IV or V,

Z is cyano, 5-membered heteroaryl, or —C(O)O-C1-6 alkyl;
W is heterocyclyl, -L-heterocyclyl, —O-L-heterocyclyl, —(N(R1c)-L)n-N(R1a)R1b, —(N(R1c)-L)n-heterocyclyl, —NHR1a, or —N(R1a)R1b;
each L is independently C1-6 alkylene or C3-7 cycloalkylene;
R6 is hydrogen, methyl, benzyl, -L-C6-14 aryl, or -L-heteroaryl;
each n is independently an integer of 1; and
R1a, R1b, and R1c are each as defined herein; wherein each alkylene, cycloalkylene, aryl, benzyl, heteroaryl, and heterocyclyl is optionally substituted with one or more substituents Q as defined herein.

In one embodiment, in Formula IV or V, W is -L-N(R1a)R1b, -L-(N(R1c)-L)n-N(R1a)R1b, —O-L-N(R1a)R1b, —O-L-(N(R1c)-L)n-N(R1a)R1b, —S-L-N(R1a)R1b, —S-L-(N(R1c)-L)n-N(R1a)R1b, or —(N(R1c)-L)n-N(R1a)R1b; and R6, R1a, R1b, R1c, L, and Z are each as defined herein.

In yet another embodiment, the stem cell mobilizing compound is a compound of Formula VI:

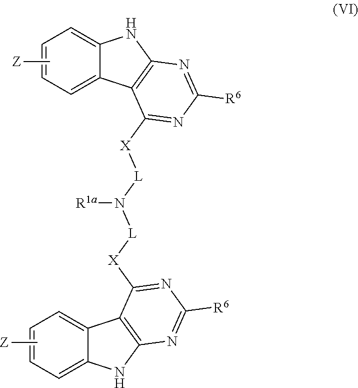

or an enantiomer, a mixture of enantiomers, a mixture of two or more diastereomers, or an isotopic variant thereof, or a pharmaceutically acceptable salt, solvate, hydrate, or prodrug thereof, wherein X is a bond, O, S, or NR1c; and R1a, R1c, R6, L, and Z are each as defined herein.

In still another embodiment, the stem cell mobilizing compound is a compound of Formula VII:

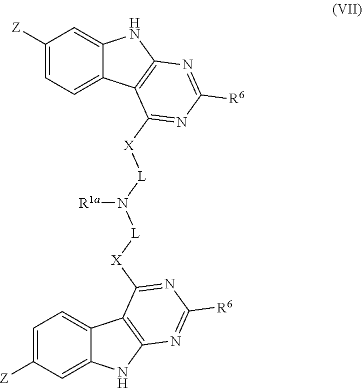

or an enantiomer, a mixture of enantiomers, a mixture of two or more diastereomers, or an isotopic variant thereof; or a pharmaceutically acceptable salt, solvate, hydrate, or prodrug thereof; wherein R1a, R6, L, X, and Z are each as defined herein.

In yet another embodiment, the stem cell mobilizing compound is a compound having the structure of:

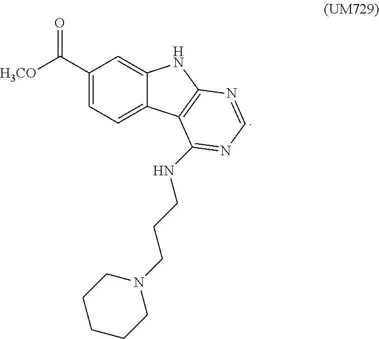 [SMILES: COC(=O)c1ccc2c(c1)[nH]c1ncnc(NCCCN3CCCCC3)c12]

In yet another embodiment, the stem cell mobilizing compound is a compound having the structure of:

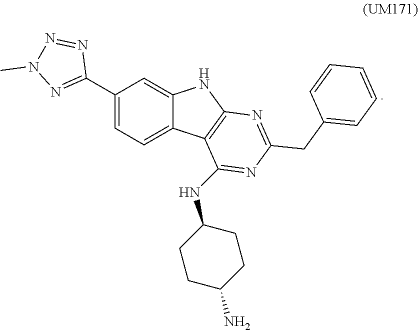 [SMILES: Cn1nnc(-c2ccc3c(c2)[nH]c2nc(Cc4ccccc4)nc(N[C@H]4CC[C@H](N)CC4)c23)n1]

In yet another embodiment, the stem cell mobilizing compound is resveratrol, tetraethylenepentamine (TEPA), alpha naphthoflavone, 3′-methoxy-4′-nitroflavone, 3,4-dimethoxyflavone, 4′,5,7-trihydroxyflavone (apigenin), 6-methyl-1,3,8-trichlorodibenzofuran, epigallocatechin, or epigallocatechingallate.

In yet another embodiment, the stem cell mobilizing compound is resveratrol. In certain embodiments, the stem cell mobilizing compound is (Z)-resveratrol. In certain embodiments, the stem cell mobilizing compound is (E)-resveratrol.

In still another embodiment, the stem cell mobilizing compound is tetraethylenepentamine (TEPA).

All of the compounds described herein are either commercially available or can be prepared according to the methods described in the patents or patent publications disclosed herein. Further, optically pure compounds can be asymmetrically synthesized or resolved using known resolving agents or chiral columns as well as other standard synthetic organic chemistry techniques. Additional information on stem cell mobilizing compounds, their preparation, and use can be found, for example, in U.S. Pat. App. Pub. Nos. 2010/0183564, 2014/0023626, and 2014/0114070; and Kim et al., Mol. Pharmacol., 2006, 69, 1871-1878; the disclosure of each of which is incorporated by reference herein in its entirety.

The groups or variables, G1, G2, G3, G4, R1, R2, R3, R4, R5a, R5b, R5c, R6, X, L, L1, X, W, Z, and n, in Formulae provided herein, e.g., Formulae I to VII, are further defined in the embodiments described herein. All combinations of the embodiments provided herein for such groups and/or variables are within the scope of this disclosure.

In certain embodiments, G1 is N. In certain embodiments, G1 is CR3, wherein R3 is as defined herein. In certain embodiments, G1 is CH.

In certain embodiments, G2 is N. In certain embodiments, G2 is CH.

In certain embodiments, G3 is N. In certain embodiments, G3 is CH.

In certain embodiments, G4 is N. In certain embodiments, G4 is CH.

In certain embodiments, R1 is hydrogen. In certain embodiments, R1 is phenyl optionally substituted as described herein. In certain embodiments, R1 is furanyl optionally substituted as described herein. In certain embodiments, R1 is pyrrolyl optionally substituted as described herein. In certain embodiments, R1 is imidazolyl optionally substituted as described herein. In certain embodiments, R1 is pyrazolyl optionally substituted as described herein. In certain embodiments, R1 is thienyl optionally substituted as described herein. In certain embodiments, R1 is thiazolyl optionally substituted as described herein. In certain embodiments, R1 is pyridinyl optionally substituted as described herein. In certain embodiments, R1 is pyrimidinyl optionally substituted as described herein. In certain embodiments, R1 is pyrrolidinyl optionally substituted as described herein. In certain embodiments, R1 is pyrazinyl optionally substituted as described herein. In certain embodiments, R1 is pyridazinyl optionally substituted as described herein. In certain embodiments, R1 is benzoimidazolyl optionally substituted as described herein. In certain embodiments, R1 is isoquinolinyl optionally substituted as described herein. In certain embodiments, R1 is imidazopyridinyl optionally substituted as described herein. In certain embodiments, R1 is benzothienyl optionally substituted as described herein.

In certain embodiments, R2 is —NR1aC(O)R1c, wherein R1a and R1c are each as defined herein. In certain embodiments, R2 is —NR1cC(O)NR1aR1b, wherein R1a, R1b, and R1c are each as defined herein. In certain embodiments, R2 is —S(O)2NR1aR1b, wherein R1a and R1b are each as defined herein. In certain embodiments, R2 is phenyl optionally substituted as described herein. In certain embodiments, R2 is pyrrolopyridin-3-yl optionally substituted as described herein. In certain embodiments, R2 is indolyl optionally substituted as described herein. In certain embodiments, R2 is thienyl optionally substituted as described herein. In certain embodiments, R2 is pyridinyl optionally substituted as described herein. In certain embodiments, R2 is 1,2,4-triazolyl optionally substituted as described herein. In certain embodiments, R2 is 2-oxoimidazolidinyl optionally substituted as described herein. In certain embodiments, R2 is pyrazolyl optionally substituted as described herein. In certain embodiments, R2 is 2-oxo-2,3-dihydro-1H-benzoimidazolyl optionally substituted as described herein. In certain embodiments, R2 is indazolyl optionally substituted as described herein.

In certain embodiments, R3 is hydrogen. In certain embodiments, R3 is C1-4 alkyl, optionally substituted with one or more substituents Q as described herein. In certain embodiments, R3 is biphenyl, optionally substituted with one or more substituents Q as described herein.

In certain embodiments, R4 is C1-10 alkyl optionally substituted as described herein. In certain embodiments, R4 is prop-1-en-2-yl optionally substituted as described herein. In certain embodiments, R4 is cyclohexyl optionally substituted as described herein. In certain embodiments, R4 is cyclopropyl optionally substituted as described herein. In certain embodiments, R4 is 2-(2-oxopyrrolidin-1-yl)ethyl optionally substituted as described herein. In certain embodiments, R4 is oxetan-3-yl optionally substituted as described herein. In certain embodiments, R4 is benzhydryl optionally substituted as described herein. In certain

embodiments, R4 is tetrahydro-2H-pyran-3-yl optionally substituted as described herein. In certain embodiments, R4 is tetrahydro-2H-pyran-4-yl optionally substituted as described herein. In certain embodiments, R4 is phenyl optionally substituted as described herein. In certain embodiments, R4 is tetrahydrofuran-3-yl optionally substituted as described herein. In certain embodiments, R4 is benzyl optionally substituted as described herein. In certain embodiments, R4 is (4-pentylphenyl)(phenyl)methyl optionally substituted as described herein. In certain embodiments, R4 is 1-(1-(2-oxo-6,9,12-trioxa-3-azatetradecan-14-yl)-1H-1,2,3-triazol-4-yl)ethyl optionally substituted as described herein.

In certain embodiments, L1 is —NR1a-, wherein R1a is as defined herein. In certain embodiments, L1 is —NR1a(CH2)1-3-, wherein R1a is as defined herein. In certain embodiments, L1 is —NR1aCH(C(O)OCH3)CH2-, wherein R1a is as defined herein. In certain embodiments, L1 is —NR1a(CH2)2NR1c-, wherein R1a and R1c are each as defined herein. In certain embodiments, L1 is —NR1a(CH2)2S—, wherein R1a is as defined herein. In certain embodiments, L1 is —NR1aCH2CH(CH3)CH2-, wherein R1a is as defined herein. In certain embodiments, L1 is —NR1aCH2CH(OH)—, wherein R1a is as defined herein. In certain embodiments, L1 is —NR1aCH(CH3)CH2-, wherein R1a is as defined herein.

In certain embodiments, R5a is hydrogen. In certain embodiments, R5a is cyano. In certain embodiments, R5a is methyl. In certain embodiments, R5a is halo. In certain embodiments, R5a is fluoro, chloro, or bromo. In certain embodiments, R5a is trifluoromethyl. In certain embodiments, R5a is —SO2CH3.

In certain embodiments, R5b is hydrogen. In certain embodiments, R5b is cyano. In certain embodiments, R5b is methyl. In certain embodiments, R5b is halo. In certain embodiments, R5b is fluoro, chloro, or bromo. In certain embodiments, R5b is trifluoromethyl. In certain embodiments, R5b is —SO2CH3.

In certain embodiments, R5c is hydrogen. In certain embodiments, R5c is cyano. In certain embodiments, R5c is methyl. In certain embodiments, R5c is halo. In certain embodiments, R5c is fluoro, chloro, or bromo. In certain embodiments, R5c is trifluoromethyl. In certain embodiments, R5c is —SO2CH3.

In certain embodiments, L is C1-6 alkylene, optionally substituted with one or more substituents Q as described herein. In certain embodiments, L is ethylene, propylene, or butylenes, each optionally substituted with one or more substituents Q as described herein. In certain embodiments, L is C2-6 alkenylene, optionally substituted with one or more substituents Q as described herein. In certain embodiments, L is C2-6 alkynylene, optionally substituted with one or more substituents Q as described herein. In certain embodiments, L is C3-7 cycloalkylene, optionally substituted with one or more substituents Q as described herein.

In certain embodiments, L is cyclohexylene, optionally substituted with one or more substituents Q as described herein. In certain embodiments, L is C6-14 arylene, optionally substituted with one or more substituents Q as described herein. In certain embodiments, L is heteroarylene, optionally substituted with one or more substituents Q as described herein. In certain embodiments, L is heterocyclylene, optionally substituted with one or more substituents Q as described herein. In certain embodiments, L is C1-6 alkylene-C3-7 cycloalkylene, optionally substituted with one or more substituents Q as described herein. In certain embodiments, L is C1-6 alkylene-heterocyclylene, optionally substituted with one or more substituents Q as described herein.

In certain embodiments, R6 is hydrogen. In certain embodiments, R6 is C1-6 alkyl, optionally substituted with one or more substituents Q as described herein. In certain embodiments, R6 is methyl, optionally substituted with one or more substituents Q as described herein. In certain embodiments, R6 is C6-14 aryl, optionally substituted with one or more substituents Q as described herein. In certain embodiments, R6 is benzyl, optionally substituted with one or more substituents Q as described herein. In certain embodiments, R6 is heteroaryl, optionally substituted with one or more substituents Q as described herein. In certain embodiments, R6 is —C(O)R1a, where R1a is as defined herein. In certain embodiments, R6 is —SR1a, where R1a is as defined herein. In certain embodiments, R6 is —S(O)R1a, where R1a is as defined herein. In certain embodiments, R6 is —S(O)2R1a, where R1a is as defined herein. In certain embodiments, R6 is -L-C6-14 aryl, where L is as defined herein. In certain embodiments, R6 is -L-heteroaryl, where L is as defined herein. In certain embodiments, R6 is or -L-heterocyclyl, where L is as defined herein.

In certain embodiments, W is hydrogen. In certain embodiments, W is halo. In certain embodiments, W is cyano. In certain embodiments, W is C6-14 aryl, optionally substituted with one or more substituents Q as described herein. In certain embodiments, W is benzyl, optionally substituted with one or more substituents Q as described herein. In certain embodiments, W is heteroaryl, optionally substituted with one or more substituents Q as described herein. In certain embodiments, W is heterocyclyl, optionally substituted with one or more substituents Q as described herein.

In certain embodiments, W is -L-C6-14 aryl, optionally substituted with one or more substituents Q as described herein, where L is as defined herein. In certain embodiments, W is -L-heteroaryl, optionally substituted with one or more substituents Q as described herein, where L is as defined herein. In certain embodiments, W is -L-heterocyclyl, optionally substituted with one or more substituents Q as described herein, where L is as defined herein. In certain embodiments, W is -L-OH, where L is as defined herein. In certain embodiments, W is -L-OR1a, where R1a and L are each as defined herein. In certain embodiments, W is -L-NH2, where L is as defined herein. In certain embodiments, W is -L-NHR1a, where R1a and L are each as defined herein. In certain embodiments, W is -L-N(R1a)R1b, where R1a, Rib, and L are each as defined herein. In certain embodiments, W is -L-SR1a, where R1a and L are each as defined herein. In certain embodiments, W is -L-S(O)R1a, where R1a and L are each as defined herein. In certain embodiments, W is -L-S(O)2R1a, where R1a and L are each as defined herein. In certain embodiments, W is -L-P(O)(OR1a)(OR1c), where R1a, R1c, and L are each as defined herein.

In certain embodiments, W is -L-(N(R1c)-L)n-N(R1a)R1b, where R1a, R1b, R1c, L and n are each as defined herein. In certain embodiments, W is -L-(N(R1c)-L)n-C6-14 aryl, optionally substituted with one or more substituents Q as described herein, where R1c, L, and n are each as defined herein. In certain embodiments, W is -L-(N(R1c)-L)n-heteroaryl, optionally substituted with one or more substituents Q as described herein, where R1c, L, and n are each as defined herein. In certain embodiments, W is -L-(N(R1c)-L)n-heterocyclyl, optionally substituted with one or more substituents Q as described herein, where R1c, L, and n are each as defined herein.

In certain embodiments, W is —O-L-N(R1a)R1b, where R1a, R1b, and L are each as defined herein. In certain embodiments, W is —O-L-C6-14 aryl, optionally substituted with one or more substituents Q as described herein, where L is as defined herein. In certain embodiments, W is —O-L-heteroaryl, optionally substituted with one or more substituents Q as described herein, where L is as defined herein. In certain embodiments, W is —O-L-heterocyclyl, optionally substituted with one or more substituents Q as described herein, where L is as defined herein.

In certain embodiments, W is —O-L-(N(R1c)-L)n-N(R1a)R1b, where R1a, R1b, R1c, L, and n are each as defined herein. In certain embodiments, W is —O-L-(N(R1c)-L)n-C6-14 aryl, optionally substituted with one or more substituents Q as described herein, where R1c, L, and n are each as defined herein. In certain embodiments, W is —O-L-(N(R1c)-L)n-heteroaryl, optionally substituted with one or more substituents Q as described herein, where R1c, L, and n are each as defined herein. In certain embodiments, W is —O-L-(N(R1c)-L)n-heterocyclyl, optionally substituted with one or more substituents Q as described herein, where R1c, L, and n are each as defined herein.

In certain embodiments, W is —S-L-N(R1a)R1b, where R1a, R1b, and L are each as defined herein. In certain embodiments, W is —S-L-C6-14 aryl, optionally substituted with one or more substituents Q as described herein, where L is as defined herein. In certain embodiments, W is —S-L-heteroaryl, optionally substituted with one or more substituents Q as described herein, where L is as defined herein. In certain embodiments, W is —S-L-heterocyclyl, optionally substituted with one or more substituents Q as described herein, where L is as defined herein.

In certain embodiments, W is —S-L-(N(R1c)-L)n-N(R1a)R1b, where R1a, R1b, R1c, L, and n are each as defined herein. In certain embodiments, W is —S-L-(N(R1c)-L)n-C6-14 aryl, optionally substituted with one or more substituents Q as described herein, where R1c, L, and n are each as defined herein. In certain embodiments, W is —S-L-(N(R1c)-L)n-heteroaryl, optionally substituted with one or more substituents Q as described herein, where R1c, L, and n are each as defined herein. In certain embodiments, W is —S-L-(N(R1c)-L)n-heterocyclyl, optionally substituted with one or more substituents Q as described herein, where R1c, L, and n are each as defined herein.

In certain embodiments, W is —(N(R1c)-L)n-N(R1a)R1b, where R1a, R1b, R1c, L, and n are each as defined herein. In certain embodiments, W is —(N(R1c)-L)n-C6-14 aryl, optionally substituted with one or more substituents Q as described herein, where R1c, L, and n are each as defined herein. In certain embodiments, W is —(N(R1c)-L)n-heteroaryl, optionally substituted with one or more substituents Q as described herein, where R1c, L, and n are each as defined herein. In certain embodiments, W is —(N(R1c)-L)n-heterocyclyl, optionally substituted with one or more substituents Q as described herein, where R1c, L, and n are each as defined herein.

In certain embodiments, W is —C(O)R1a, where R1a is as defined herein. In certain embodiments, W is —C(O)OR1a, where R1a is as defined herein. In certain embodiments, W is —C(O)NH2. In certain embodiments, W is —C(O)NHR1a, where R1a is as defined herein. In certain embodiments, W is —C(O)N(R1a)R1b, where R1a and Rib are each as defined herein.

In certain embodiments, W is —NHR1a, where R1a is as defined herein. In certain embodiments, W is —N(R1a)R1b, where R1a and Rib are each as defined herein. In certain embodiments, W is —NHC(O)R1a, where R1a is as defined herein. In certain embodiments, W is —NR1aC(O)R1c, where R1a and R1c are each as defined herein. In certain embodiments, W is —NHC(O)OR1a, where R1a is as defined herein. In certain embodiments, W is —NR1aC(O)OR1c, where R1a and R1c are each as defined herein. In certain embodiments, W is-NHC(O)NH2. In certain embodiments, W is —NHC(O)NHR1a, where R1a is as defined herein. In certain embodiments, W is —NHC(O)N(R1a)R1b, where R1a and R1b are each as defined herein. In certain embodiments, W is —NR1aC(O)NH2, where R1a is as defined herein. In certain embodiments, W is —NR1cC(O)NHR1a, where R1a and R1c are each as defined herein. In certain embodiments, W is —NR1cC(O)N(R1a)R1b, where R1a, R1b, and R1c are each as defined herein. In certain embodiments, W is —NHS(O)2R1a, where R1a is as defined herein. In certain embodiments, W is —NR1cS(O)2R1a, where R1a and R1c are each as defined herein. In certain embodiments, W is —OR1a, where R1a is as defined herein. In certain embodiments, W is —OC(O)R1a, where R1a is as defined herein. In certain embodiments, W is —OC(O)OR1a, where R1a is as defined herein. In certain embodiments, W is-OC(O)NH2. In certain embodiments, W is —OC(O)NHR1a, where R1a is as defined herein. In certain embodiments, W is —OC(O)N(R1a)R1b, where R1a and R1b are each as defined herein. In certain embodiments, W is —OS(O)2R1a, where R1a is as defined herein. In certain embodiments, W is —P(O)(OR1a)(OR1c), where R1a and R1c are each as defined herein. In certain embodiments, W is —SR1a, where R1a is as defined herein. In certain embodiments, W is-S(O)R1a, where R1a is as defined herein. In certain embodiments, W is —S(O)2R1a, where R1a is as defined herein. In certain embodiments, W is —S(O)2NH2. In certain embodiments, W is —S(O)2NHR1a, where R1a is as defined herein. In certain embodiments, W is —S(O)2N(R1a)R1b, where R1a and Rib are each as defined herein. In certain embodiments, W is —S(O)2OR1a, where R1a is as defined herein.

In certain embodiments, Z is cyano. In certain embodiments, Z is C1-6 alkyl, optionally substituted with one or more substituents Q as described herein. In certain

embodiments, Z is C2-6 alkenyl, optionally substituted with one or more substituents Q as described herein. In certain embodiments, Z is C2-6 alkynyl, optionally substituted with one or more substituents Q as described herein. In certain embodiments, Z is C3-10 cycloalkyl, optionally substituted with one or more substituents Q as described herein. In certain embodiments, Z is C6-14 aryl, optionally substituted with one or more substituents Q as described herein. In certain embodiments, Z is C7-15 aralkyl, optionally substituted with one or more substituents Q as described herein. In certain embodiments, Z is benzyl, optionally substituted with one or more substituents Q as described herein. In certain embodiments, Z is heteroaryl, optionally substituted with one or more substituents Q as described herein. In certain embodiments, Z is 5-membered heteroaryl, optionally substituted with one or more substituents Q as described herein. In certain embodiments, Z is tetrazolyl, optionally substituted with one or more substituents Q as described herein. In certain embodiments, Z is 1,2,4-oxadiazolyl, optionally substituted with one or more substituents Q as described herein. In certain embodiments, Z is heterocyclyl, optionally substituted with one or more substituents Q as described herein. In certain embodiments, Z is -L-C6-14 aryl, optionally substituted with one or more substituents Q as described herein, where L is as defined herein. In certain embodiments, Z is -L-heteroaryl, optionally substituted with one or more substituents Q as described herein, where L is as defined herein. In certain embodiments, Z is -L-heterocyclyl, optionally substituted with one or more substituents Q as described herein, where L is as defined herein.

In certain embodiments, Z is —C(O)R1a, wherein R1a is as defined herein. In certain embodiments, Z is —C(O)OR1a, wherein R1a is as defined herein. In certain embodiments, Z is —C(O)OC1-6 alkyl, wherein the alkyl is optionally substituted with one or more substituents Q as defined herein. In certain embodiments, Z is —C(O)OCH3. In certain embodiments, Z is —C(O)NHR1a, wherein R1a is as defined herein. In certain embodiments, Z is —C(O)N(R1a)R1b, wherein R1a and Rib are each as defined herein. In certain embodiments, Z is —P(O)(OR1a)(OR1c), wherein R1a and R1c are each as defined herein. In certain embodiments, Z is —SR1a, wherein R1a is as defined herein. In certain embodiments, Z is —S(O)R1a, wherein R1a is as defined herein. In certain embodiments, Z is —S(O)2R1a, wherein R1a is as defined herein. In certain embodiments, Z is —S(O)2NH2. In certain embodiments, Z is —S(O)2NHR1a, wherein R1a is as defined herein. In certain embodiments, Z is —S(O)2N(R1a)R1b, wherein R1a and Rib are each as defined herein.

In certain embodiments, X is a bond. In certain embodiments, X is O. In certain embodiments, X is S. In certain embodiments, X is NR1c, where R1c is as defined herein.

In certain embodiments, n is 1. In certain embodiments, n is 2. In certain embodiments, n is 3. In certain embodiments, n is 4. In certain embodiments, n is 5.

In certain embodiments, the compounds provided herein show activity as antagonists of an AHR.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

### 5.6.7. Treatment of NK Cells with an Immunomodulatory Compound

Isolated NK cells, e.g., PINK cells or combined NK cells, as described elsewhere herein, can be treated with an immunomodulatory compound, e.g., contacted with an immunomodulatory compound, to enhance the antitumor activity of the cell. Thus, provided herein is a method of increasing the cytotoxicity of a NK cell against a tumor cell comprising contacting the NK cell with an immunomodulatory compound for a time and in a concentration sufficient for the NK cell to demonstrate increased cytotoxicity towards a tumor cell compared to a NK cell not contacted with the immunomodulatory compound. In another embodiment, provided herein is a method of increasing the expression of granzyme B in a NK cell comprising contacting the NK cell with an immunomodulatory compound for a time and in a concentration sufficient for the NK cell to demonstrate increased expression of granzyme B compared to a NK cell not contacted with the immunomodulatory compound. The immunomodulatory compound can be any immunomodulatory compound described below, e.g., lenalidomide, pomalidomide, or thalidomide.

Also provided herein is a method of increasing the cycotoxicity of a population of NK cells, e.g., PINK cells or combined NK cells, to a plurality of tumor cells comprising contacting the population of NK cells with an immunomodulatory compound for a time and in a concentration sufficient for the population of NK cells to demonstrate detectably increased cytotoxicity towards said plurality of tumor cells compared to an equivalent number of NK cells not contacted with the immunomodulatory compound. In another embodiment, provided herein is a method of increasing the expression of granzyme B in a population of NK cells comprising contacting the population of NK cells with an immunomodulatory compound for a time and in a concentration sufficient for the population of NK cells to express a detectably increased amount of granzyme B compared to an equivalent number of NK cells not contacted with the immunomodulatory compound. In a specific embodiment, said population of NK cells is contained within placental perfusate cells, e.g., total nucleated cells from placental perfusate.

In specific embodiments of the above embodiments, the NK cells are CD56⁺, CD16⁻ PINK cells. In another specific embodiment of the above embodiments, the NK cells are combined NK cells, i.e., NK cells from matched placental perfusate and umbilical cord blood.

In another specific embodiment, said plurality of NK cells, e.g., PINK cells or combined NK cells, contacted with said immunomodulatory compound express one or more of BAX, CCL5, CCR5, CSF2, FAS, GUSB, IL2RA, or TNFRSF18 at a higher level than an equivalent number of said NK cells not contacted with said immunomodulatory compound. In another specific embodiment, said plurality of NK cells, e.g., PINK cells, contacted with said immunomodulatory compound express one or more of ACTB, BAX, CCL2, CCL3, CCL5, CCR5, CSF1, CSF2, ECE1, FAS, GNLY, GUSB, GZMB, IL1A, IL2RA, IL8, IL10, LTA, PRF1, PTGS2, SKI, and TBX21 at a higher level than an equivalent number of said NK cells not contacted with said immunomodulatory compound.

Also provided herein is a method of increasing the cycotoxicity of a population of human placental perfusate cells, e.g., total nucleated cells from placental perfusate, towards a plurality of tumor cells, comprising contacting the placental perfusate cells with an immunomodulatory compound for a time and in a concentration sufficient for the placental perfusate cells to demonstrate detectably increased cytotoxicity towards said plurality of tumor cells compared to an equivalent number of placental perfusate cells not contacted with the immunomodulatory compound. In another embodiment, provided herein is a method of increasing the expression of granzyme B in a population of placental perfusate cells comprising contacting the population of placental perfusate cells with an immunomodulatory compound for a time and in a concentration sufficient for the population of placental perfusate cells to express a detectably increased amount of granzyme B compared to an equivalent number of placental perfusate cells not contacted with the immunomodulatory compound.

Immunomodulatory compounds can either be commercially purchased or prepared according to the methods described in the patents or patent publications referred to herein, all of which are incorporated by reference. Further, optically pure compositions can be asymmetrically synthesized or resolved using known resolving agents or chiral columns as well as other standard synthetic organic chemistry techniques. Immunomodulatory compounds may be racemic, stereomerically enriched or stereomerically pure, and may encompass pharmaceutically acceptable salts, solvates, and prodrugs thereof.

As used herein and unless otherwise indicated, the terms “immunomodulatory compounds” encompass small organic molecules that markedly inhibit TNF-α, LPS induced monocyte IL-1β and IL-12, and partially inhibit IL-6 production. In specific examples, the immunomodulatory compounds are lenalidomide, pomalidomide or thalidomide.

Specific examples of immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent publication no. 2003/0045552 A1, U.S. Pat. No. 7,091,353, and WO 02/059106. The entireties of each of the patents and patent applications identified herein are incorporated herein by reference. Immunomodulatory compounds do not include thalidomide.

In certain embodiments, the immunomodulatory compounds are 1-oxo- and 1,3 dioxo-2-(2,6-dioxopiperidin-3-yl) isoindolines substituted with amino in the benzo ring as described in U.S. Pat. No. 5,635,517, which is incorporated herein by reference in its entirety. These compounds have the structure I:

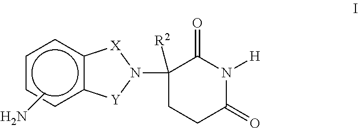

in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline: and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and WO 98/03502, each of which is incorporated herein by reference. Representative compounds are of formula:

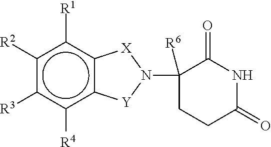

in which:

one of X and Y is C═O and the other of X and Y is C═O or CH2;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R²,

R³, and R⁴ is fluoro or (ii) one of R¹, R², R³, or R⁴ is amino.

Compounds representative of this class are of the formulas:

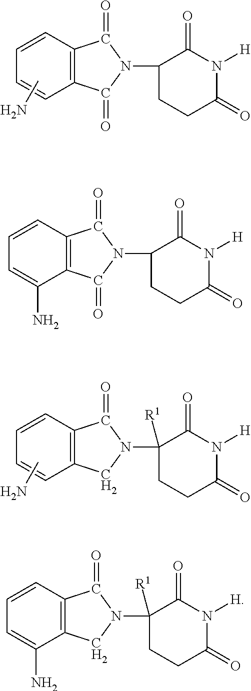

wherein R¹ is hydrogen or methyl. In a separate embodiment, encompassed is the use of enantiomerically pure forms (e.g. optically pure (R) or (S) enantiomers) of these compounds.

Still other specific immunomodulatory compounds belong to a class of isoindole-imides disclosed in U.S. Patent Application Publication Nos. US 2003/0096841 and US 2003/0045552, and WO 02/059106, each of which are incorporated herein by reference. Representative compounds are of formula II:

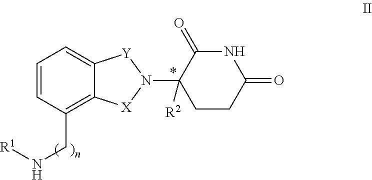

and pharmaceutically acceptable salts, hydrates, solvates, clathrates, enantiomers, diastereomers, racemates, and mixtures of stereoisomers thereof, wherein:

one of X and Y is C═O and the other is CH2 or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₈)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₈)heteroaryl, or (C₀-C₈)alkyl-C(O)O—R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

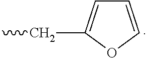

In another embodiment of the compounds of formula II, R¹ is

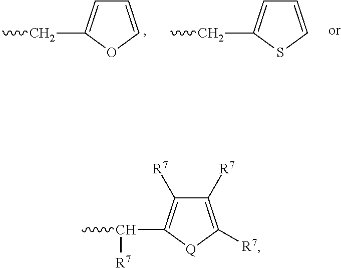

wherein Q is O or S, and each occurrence of R⁷ is independently H, (C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₈)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₅)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6-dioxo(3-piperidyl))-1,3-dioxoisoindolin-4-yl] methyl}(benzylamino)carboxamide.

Still other specific immunomodulatory compounds belong to a class of isoindole-imides disclosed in U.S. Patent Application Publication No. 2002/0045643, International Publication No. WO 98/54170, and U.S. Pat. No. 6,395,754, each of which is incorporated herein by reference. Representative compounds are of formula III:

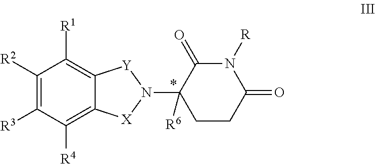

and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

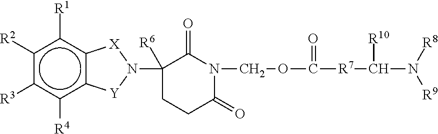

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—;

R¹⁰ is hydrogen, alkyl of to 8 carbon atoms, or phenyl.

Other representative compounds are of formula:

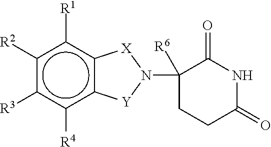

in which

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and R⁶ is hydrogen, alkyl of 1 to 8 carbon atoms, benzo, chloro, or fluoro.

Other representative compounds are of formula:

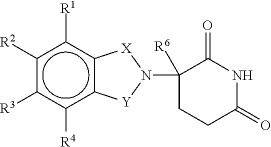

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen, alkyl of 1 to 8 carbon atoms, or CO—R⁷—CH(R¹⁰)NR⁵R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

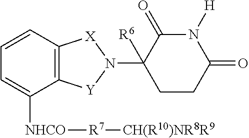

in which:

one of X and Y is C═O and the other of X and Y is C═O or CH₂;

R⁶ is hydrogen, alkyl of 1 to 8 carbon atoms, benzyl, chloro, or fluoro;

R⁷ is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

The most preferred immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure:

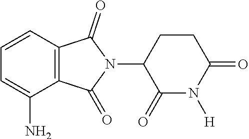 [SMILES: Nc1cccc2c1C(=O)N(C1CCC(=O)NC1=O)C2=O]

The compound 3-(4-amino-1-oxo-1,3-dihydro-isoindol-2-yl)-piperidine-2,6-dione has the following chemical structure:

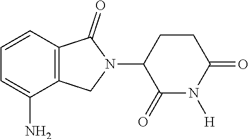 [SMILES: Nc1cccc2c1CN(C1CCC(=O)NC1=O)C2=O]

In another embodiment, specific immunomodulatory compounds encompass polymorphic forms of 3-(4-amino-1-oxo-1,3 dihydro-isoindol-2-yl)-piperidene-2,6-dione such as Form A, B, C, D, E, F, G and H, disclosed in U.S. publication no. US 2005/0096351 A1, which is incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds usable in the methods provided herein include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

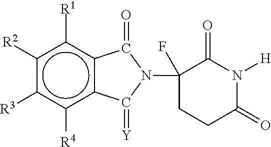

wherein Y is oxygen or H² and
each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds usable in the methods provided herein include, but are not limited to, the tetra substituted 2-(2,6-dioxopiperdin-3-yl)-1-oxoisoindolines described in U.S. Pat. No. 5,798,368, which is incorporated herein by reference. Representative compounds are of formula:

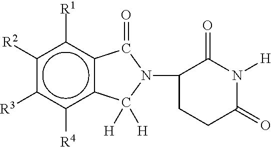

wherein each of R¹, R², R³, and R⁴, independently of the others, is halo, alkyl of 1 to 4 carbon atoms, or alkoxy of 1 to 4 carbon atoms.

Other specific immunomodulatory compounds that can be used in the methods provided herein include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

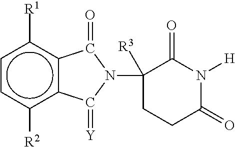

in which

Y is oxygen or H2,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

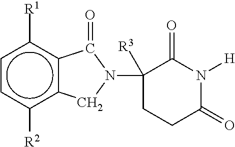

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other compounds that can be used in the methods provided herein are of formula:

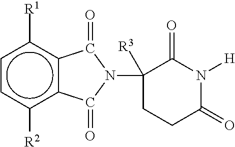

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Other specific immunomodulatory compounds that can be used in the methods provided herein include, but are not limited to, 1-oxo and 1,3-dioxoisoindolines substituted in the 4- or 5-position of the indoline ring described in U.S. Pat. No. 6,380,239 and U.S. Application Publication No. 2006/0084815, which are incorporated herein by reference. Representative compounds are of formula:

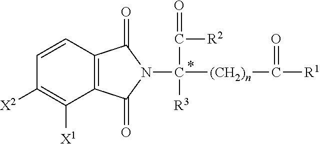

in which the carbon atom designated C* constitutes a center of chirality (when n is not zero and R¹ is not the same as R²); one of X¹ and X² is amino, nitro, alkyl of one to six carbons, or NH—Z, and the other of X¹ or X² is hydrogen; each of R¹ and R² independent of the other, is hydroxy or NH—Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further compounds that can be used in the methods provided herein are of formula:

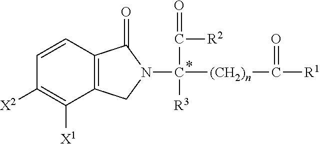

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl or an alkyl or acyl of one to six carbons; and n has a value of 0, 1, or 2.

Specific examples of compounds that can be used in the methods provided herein include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

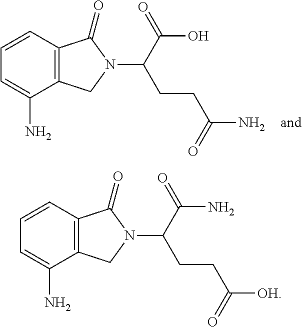

Other representative compounds are of formula:

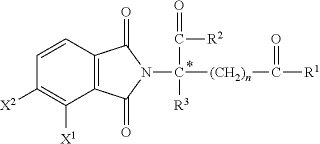

in which the carbon atom designated C* constitutes a center of chirality when n is not zero and R¹ is not R²; one of X¹ and X² is amino, nitro, alkyl of one to six carbons, or NH—Z, and the other of X¹ or X² is hydrogen; each of R¹ and R² independent of the other, is hydroxy or NH—Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(furan-2-yl-methyl)-amino]-1,3-dioxo-1,3-dihydro-isoindol-2-yl)}-4-phenylcarbamoyl-butyric acid, and 2-{4-[(furan-2-yl-methyl)-amino]-1,3-dioxo-1,3-dihydro-isoindol-2-yl}-pentanedioic acid, which have the following structures, respectively, and pharmaceutically acceptablesalts, solvate, prodrugs, and stereoisomers thereof:

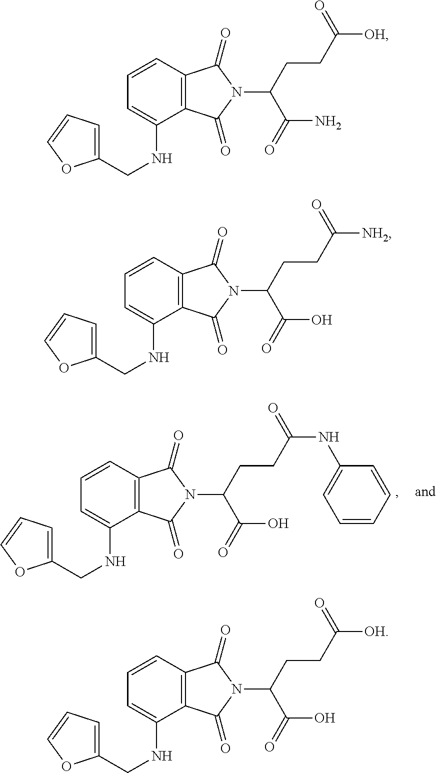 [SMILES: NC(=O)C(CCC(=O)O)N1C(=O)c2cccc(NCc3ccco3)c2C1=O ; NC(=O)CCC(C(=O)O)N1C(=O)c2cccc(NCc3ccco3)c2C1=O ; O=C(CCC(C(=O)O)N1C(=O)c2cccc(NCc3ccco3)c2C1=O)Nc1ccccc1 ; O=C(O)CCC(C(=O)O)N1C(=O)c2cccc(NCc3ccco3)c2C1=O]

Other specific examples of the compounds are of formula:

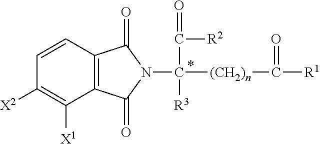

wherein one of X¹ and X² is nitro, or NH—Z, and the other of X¹ or X² is hydrogen;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH2)ₙCOR¹ are different, the carbon atom designated C* constitutes a center of chirality.

Other representative compounds are of formula:

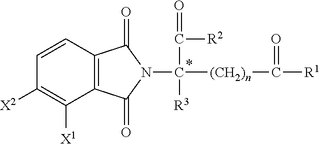

wherein one of X¹ and X² is alkyl of one to six carbons;

each of R¹ and R², independent of the other, is hydroxy or NH—Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH2)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

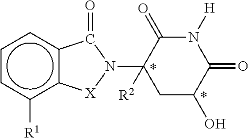

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen; and

R³ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen, alkyl of 1 to 8 carbon atoms, unsubstituted or substituted with alkoxy of 1 to 8 carbon atoms, halo, amino, or alkylamino of 1 to 4 carbon atoms, cycloalkyl of 3 to 18 carbon atoms, phenyl, unsubstituted or substituted with alkyl of 1 to 8 carbon atoms, alkoxy of 1 to 8 carbon atoms, halo, amino, or alkylamino of 1 to 4 carbon atoms, or benzyl, unsubstituted or substituted with alkyl of 1 to 8 carbon atoms, alkoxy of 1 to 8 carbon atoms, halo, amino, or alkylamino of 1 to 4 carbon atoms.

Compounds provided herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

Various immunomodulatory compounds contain one or more chiral centers, and can exist as racemic mixtures of enantiomers or mixtures of diastereomers. Encompassed is the use of stereomerically pure forms of such compounds, as well as the use of mixtures of those forms. For example, mixtures comprising equal or unequal amounts of the enantiomers of a particular immunomodulatory compounds may be used in methods and compositions provided herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 5.7. Administration of Human Placental Perfusate, Placental Perfusate Cells, PINK Cells, Combined NK Cells, or Combinations Thereof and Administration in Combination with an Antibody

The human placental perfusate, human placental perfusate cells, PINK cells, combined NK cells, populations of cells comprising such cells, or combinations thereof, and the antibody can be administered to an individual, e.g., an individual having tumor cells, e.g., a cancer patient, concurrently or sequentially. In certain embodiments, the human placental perfusate, human placental perfusate cells, PINK cells, combined NK cells, populations of cells comprising such cells, or combinations thereof, and the antibody are administered concurrently. In other embodiments, the human placental perfusate, human placental perfusate cells, PINK cells, combined NK cells, populations of cells comprising such cells, or combinations thereof, and the antibody are administered sequentially. In one embodiment, the human placental perfusate, human placental perfusate cells, PINK cells, combined NK cells, populations of cells comprising such cells, or combinations thereof, are administered before the antibody. In another embodiment, the human placental perfusate, human placental perfusate cells, PINK cells, combined NK cells, populations of cells comprising such cells, or combinations thereof, are administered after the antibody.

The human placental perfusate, human placental perfusate cells, PINK cells, combined NK cells, populations of cells comprising such cells, or combinations thereof, and the antibody may be administered via the same or different routes of administration. In certain embodiments, the human placental perfusate, human placental perfusate cells, PINK cells, combined NK cells, populations of cells comprising such cells, or combinations thereof, and the antibody are administered via the same route of administration. In other embodiments, the human placental perfusate, human placental perfusate cells, PINK cells, combined NK cells, populations of cells comprising such cells, or combinations thereof, and the antibody are administered via different routes of administration.

The human placental perfusate, human placental perfusate cells, PINK cells, combined NK cells, populations of cells comprising such cells, or combinations thereof, can be administered to an individual, e.g., an individual having tumor cells, e.g., a cancer patient, by any medically-acceptable route known in the art suitable to the administration of live cells. In various embodiments, the cells provided herein can be surgically implanted, injected, infused, e.g., by way of a catheter or syringe, or otherwise administered directly or indirectly to the site in need of repair or augmentation. In one embodiment, the cells are administered to an individual intravenously. In another embodiment, the cells are administered to an individual intracranially. In yet another embodiment, the cells are administered to an individual intrathecally. In another embodiment, the cells are administered to the individual at the site of a tumor, e.g., a solid tumor. In a specific embodiment in which the individual has a tumor at more than one site, the cells are administered to at least two, or all, tumor sites. In certain other embodiments, the cells provided herein, or compositions comprising the cells, are administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In certain specific embodiments, the cells are delivered via intracranial or intravertebral needles and/or catheters with or without pump devices.

The antibody can be administered to an individual, e.g., an individual having tumor cells, e.g., a cancer patient, by any medically-acceptable route known in the art suitable to the administration of antibody. In various embodiments, the antibody provided herein can be surgically implanted, injected, infused, e.g., by way of a catheter or syringe, or otherwise administered directly or indirectly to the site in need of repair or augmentation. In one embodiment, the antibody is administered to an individual intravenously. In another embodiment, the antibody is administered to an individual intracranially. In yet another embodiment, the antibody is administered to an individual intrathecally. In another embodiment, the antibody is administered to the individual at the site of a tumor, e.g., a solid tumor. In a specific embodiment in which the individual has a tumor at more than one site, the antibody is administered to at least two, or all, tumor sites. In certain other embodiments, the antibody is administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In certain specific embodiments, the antibody is delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, and the antibody are administered to an individual intravenously. In some embodiments, the human placental perfusate, human placental perfusate cells, PINK cells, combined NK cells, populations of cells comprising such cells, or combinations thereof, and the antibody are administered to an individual intracranially. In other embodiments, the human placental perfusate, human placental perfusate cells, PINK cells, combined NK cells, populations of cells comprising such cells, or combinations thereof, and the antibody are administered to an individual intrathecally. In certain other embodiments, the human placental perfusate, human placental perfusate cells, PINK cells, combined NK cells, populations of cells comprising such cells, or combinations thereof, and the antibody are administered orally, nasally, intraarterially, parenterally, ophthalmically, intramuscularly, subcutaneously, intraperitoneally, intracerebrally, intraventricularly, intracerebroventricularly, intracistemally, intraspinally and/or perispinally. In various embodiments, the human placental perfusate, human placental perfusate cells, PINK cells, combined NK cells, populations of cells comprising such cells, or combinations thereof, and the antibody are surgically implanted, injected, infused, e.g., by way of a catheter or syringe, or otherwise administered directly or indirectly to the site in need of repair or augmentation. In certain specific embodiments, the human placental perfusate, human placental perfusate cells, PINK cells, combined NK cells, populations of cells comprising such cells, or combinations thereof, and the antibody are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and the antibody is administered intracranially to an individual. In some embodiments, the human placental perfusate, human placental perfusate cells, PINK cells, combined NK cells, populations of cells comprising such cells, or combinations thereof, are administered intracranially, and the antibody is administered intrathecally to an individual. In other embodiments, the human placental perfusate, human placental perfusate cells, PINK cells, combined NK cells, populations of cells comprising such cells, or combinations thereof, are administered intrathecally, and the antibody is administered intravenously to an individual. In certain embodiments of various methods provided herein, the human placental perfusate, human placental perfusate cells, PINK cells, combined NK cells, populations of cells comprising such cells, or combinations thereof, are administered intravenously, and the antibody is administered intrathecally to an individual. In some embodiments, the human placental perfusate, human placental perfusate cells, PINK cells, combined NK cells, populations of cells comprising such cells, or combinations thereof, are administered intracranially, and the antibody is administered intravenously to an individual. In other embodiments, the human placental perfusate, human placental perfusate cells, PINK cells, combined NK cells, populations of cells comprising such cells, or combinations thereof, are administered intrathecally, and the antibody is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and the antibody is administered by an implanted catheter to an individual. In some embodiments, the human placental perfusate, human placental perfusate cells, PINK cells, combined NK cells, populations of cells comprising such cells, or combinations thereof, are administered intracranially, and the antibody is administered an implanted catheter to an individual. In other embodiments, the human placental perfusate, human placental perfusate cells, PINK cells, combined NK cells, populations of cells comprising such cells, or combinations thereof, are administered intrathecally, and the antibody is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and the antibody is administered intrathecally to an individual. In some embodiments, the human placental perfusate, human placental perfusate cells, PINK cells, combined NK cells, populations of cells comprising such cells, or combinations thereof, are administered an implanted catheter, and the antibody is administered intravenously to an individual. In other embodiments, the human placental perfusate, human placental perfusate cells, PINK cells, combined NK cells, populations of cells comprising such cells, or combinations thereof, are administered an implanted catheter, and the antibody is administered intracranially to an individual.

In certain embodiments, the anti-CD38 antibody and the human placental perfusate, placental perfusate cells, PINK cells, combined NK cells, or combinations thereof are administered concurrently. In other embodiments, the anti-CD38 antibody is administered before administration of the human placental perfusate, placental perfusate cells, PINK cells, combined NK cells, or combinations thereof. In yet other embodiments, the anti-CD38 antibody is administered after administration of the human placental perfusate, placental perfusate cells, PINK cells, combined NK cells, or combinations thereof. In one embodiment, the anti-CD38 antibody is daratumumab. In certain embodiments, daratumumab and the human placental perfusate, placental perfusate cells, PINK cells, combined NK cells, or combinations thereof are administered concurrently. In other embodiments, daratumumab is administered before administration of the human placental perfusate, placental perfusate cells, PINK cells, combined NK cells, or combinations thereof. In yet other embodiments, daratumumab is administered after administration of the human placental perfusate, placental perfusate cells, PINK cells, combined NK cells, or combinations thereof. In certain embodiments, daratumumab and the human placental perfusate, placental perfusate cells, PINK cells, combined NK cells, or combinations thereof are administered via the same route of administration. In other embodiments, daratumumab and the human placental perfusate, placental perfusate cells, PINK cells, combined NK cells, or combinations thereof are administered via different routes of administration. In certain embodiments, daratumumab and the human placental perfusate, placental perfusate cells, PINK cells, combined NK cells, or combinations thereof are administered intravenously. In other embodiments, daratumumab and the human placental perfusate, placental perfusate cells, PINK cells, combined NK cells, or combinations thereof are administered intracranially. In yet other embodiments, daratumumab and the human placental perfusate, placental perfusate cells, PINK cells, combined NK cells, or combinations thereof are administered intrathecally. In still other embodiments, daratumumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, daratumumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and daratumumab is administered intracranially to an individual. In some embodiments, the human placental perfusate, human placental perfusate cells, PINK cells, combined NK cells, populations of cells comprising such cells, or combinations thereof, are administered intracranially, and daratumumab is administered intrathecally to an individual. In other embodiments, the human placental perfusate, human placental perfusate cells, PINK cells, combined NK cells, populations of cells comprising such cells, or combinations thereof, are administered intrathecally, and daratumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and daratumumab is administered intrathecally to an individual. In some embodiments, the human placental perfusate, human placental perfusate cells, PINK cells, combined NK cells, populations of cells comprising such cells, or combinations thereof, are administered intracranially, and daratumumab is administered intravenously to an individual. In other embodiments, the human placental perfusate, human placental perfusate cells, PINK cells, combined NK cells, populations of cells comprising such cells, or combinations thereof, are administered intrathecally, and daratumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and daratumumab is administered by an implanted catheter to an individual. In some embodiments, the human placental perfusate, human placental perfusate cells, PINK cells, combined NK cells, populations of cells comprising such cells, or combinations thereof, are administered intracranially, and daratumumab is administered an implanted catheter to an individual. In other embodiments, the human placental perfusate, human placental perfusate cells, PINK cells, combined NK cells, populations of cells comprising such cells, or combinations thereof, are administered intrathecally, and daratumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and daratumumab is administered intrathecally to an individual. In some embodiments, the human placental perfusate, human placental perfusate cells, PINK cells, combined NK cells, populations of cells comprising such cells, or combinations thereof, are administered an implanted catheter, and daratumumab is administered intravenously to an individual. In other embodiments, the human placental perfusate, human placental perfusate cells, PINK cells, combined NK cells, populations of cells comprising such cells, or combinations thereof, are administered an implanted catheter, and daratumumab is administered intracranially to an individual.

In certain embodiments, the anti-SLAMF7 antibody and the human placental perfusate, placental perfusate cells, PINK cells, combined NK cells, or combinations thereof are administered concurrently. In other embodiments, the anti-SLAMF7 antibody is administered before administration of the human placental perfusate, placental perfusate cells, PINK cells, combined NK cells, or combinations thereof. In yet other embodiments, the anti-SLAMF7 antibody is administered after administration of the human placental perfusate, placental perfusate cells, PINK cells, combined NK cells, or combinations thereof. In one embodiment, the anti-SLAMF7 antibody is elotuzumab. In certain embodiments, elotuzumab and the human placental perfusate, placental perfusate cells, PINK cells, combined NK cells, or combinations thereof are administered concurrently. In other embodiments, elotuzumab is administered before administration of the human placental perfusate, placental perfusate cells, PINK cells, combined NK cells, or combinations thereof. In yet other embodiments, elotuzumab is administered after administration of the human placental perfusate, placental perfusate cells, PINK cells, combined NK cells, or combinations thereof. In certain embodiments, elotuzumab and the human placental perfusate, placental perfusate cells, PINK cells, combined NK cells, or combinations thereof are administered via the same route of administration. In other embodiments, elotuzumab and the human placental perfusate, placental perfusate cells, PINK cells, combined NK cells, or combinations thereof are administered via different routes of administration. In certain embodiments, elotuzumab and the human placental perfusate, placental perfusate cells, PINK cells, combined NK cells, or combinations thereof are administered intravenously. In other embodiments, elotuzumab and the human placental perfusate, placental perfusate cells, PINK cells, combined NK cells, or combinations thereof are administered intracranially. In yet other embodiments, elotuzumab and the human placental perfusate, placental perfusate cells, PINK cells, combined NK cells, or combinations thereof are administered intrathecally. In still other embodiments, elotuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, elotuzumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and elotumumab is administered intracranially to an individual. In some embodiments, the human placental perfusate, human placental perfusate cells, PINK cells, combined NK cells, populations of cells comprising such cells, or combinations thereof, are administered intracranially, and elotumumab is administered intrathecally to an individual. In other embodiments, the human placental perfusate, human placental perfusate cells, PINK cells, combined NK cells, populations of cells comprising such cells, or combinations thereof, are administered intrathecally, and elotumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and elotumumab is administered intrathecally to an individual. In some embodiments, the human placental perfusate, human placental perfusate cells, PINK cells, combined NK cells, populations of cells comprising such cells, or combinations thereof, are administered intracranially, and elotumumab is administered intravenously to an individual. In other embodiments, the human placental perfusate, human placental perfusate cells, PINK cells, combined NK cells, populations of cells comprising such cells, or combinations thereof, are administered intrathecally, and elotumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and elotumumab is administered by an implanted catheter to an individual. In some embodiments, the human placental perfusate, human placental perfusate cells, PINK cells, combined NK cells, populations of cells comprising such cells, or combinations thereof, are administered intracranially, and elotumumab is administered an implanted catheter to an individual. In other embodiments, the human placental perfusate, human placental perfusate cells, PINK cells, combined NK cells, populations of cells comprising such cells, or combinations thereof, are administered intrathecally, and elotumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and elotumumab is administered intrathecally to an individual. In some embodiments, the human placental perfusate, human placental perfusate cells, PINK cells, combined NK cells, populations of cells comprising such cells, or combinations thereof, are administered an implanted catheter, and elotumumab is administered intravenously to an individual. In other embodiments, the human placental perfusate, human placental perfusate cells, PINK cells, combined NK cells, populations of cells comprising such cells, or combinations thereof, are administered an implanted catheter, and elotumumab is administered intracranially to an individual.

In certain embodiments, the anti-CD20 antibody and the human placental perfusate, placental perfusate cells, PINK cells, combined NK cells, or combinations thereof are administered concurrently. In other embodiments, the anti-CD20 antibody is administered before administration of the human placental perfusate, placental perfusate cells, PINK cells, combined NK cells, or combinations thereof. In yet other embodiments, the anti-CD20 antibody is administered after administration of the human placental perfusate, placental perfusate cells, PINK cells, combined NK cells, or combinations thereof. In one embodiment, the anti-CD20 antibody is obinutuzumab. In another embodiment, the anti-CD20 antibody is ofatumumab. In yet another embodiment, the anti-CD20 antibody is rituximab. In certain embodiments, obinutuzumab and the human placental perfusate, placental perfusate cells, PINK cells, combined NK cells, or combinations thereof are administered concurrently. In other embodiments, obinutuzumab is administered before administration of the human placental perfusate, placental perfusate cells, PINK cells, combined NK cells, or combinations thereof. In yet other embodiments, obinutuzumab is administered after administration of the human placental perfusate, placental perfusate cells, PINK cells, combined NK cells, or combinations thereof. In certain embodiments, obinutuzumab and the human placental perfusate, placental perfusate cells, PINK cells, combined NK cells, or combinations thereof are administered via the same route of administration. In other embodiments, obinutuzumab and the human placental perfusate, placental perfusate cells, PINK cells, combined NK cells, or combinations thereof are administered via different routes of administration. In certain embodiments, obinutuzumab and the human placental perfusate, placental perfusate cells, PINK cells, combined NK cells, or combinations thereof are administered intravenously. In other embodiments, obinutuzumab and the human placental perfusate, placental perfusate cells, PINK cells, combined NK cells, or combinations thereof are administered intracranially. In yet other embodiments, obinutuzumab and the human placental perfusate, placental perfusate cells, PINK cells, combined NK cells, or combinations thereof are administered intrathecally. In still other embodiments, obinutuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, obinutuzumab and the human placental perfusate, placental perfusate cells, PINK cells, combined NK cells, or combinations thereof are delivered via intracranial or intravertebral needles and/or catheters with or without pump devices. In certain embodiments, ofatumumab and the human placental perfusate, placental perfusate cells, PINK cells, combined NK cells, or combinations thereof are administered concurrently. In other embodiments, ofatumumab is administered before administration of the human placental perfusate, placental perfusate cells, PINK cells, combined NK cells, or combinations thereof. In yet other embodiments, ofatumumab is administered after administration of the human placental perfusate, placental perfusate cells, PINK cells, combined NK cells, or combinations thereof. In certain embodiments, ofatumumab and the human placental perfusate, placental perfusate cells, PINK cells, combined NK cells, or combinations thereof are administered via the same route of administration. In other embodiments, ofatumumab and the human placental perfusate, placental perfusate cells, PINK cells, combined NK cells, or combinations thereof are administered via different routes of administration. In certain embodiments, ofatumumab and the human placental perfusate, placental perfusate cells, PINK cells, combined NK cells, or combinations thereof are administered intravenously. In other embodiments, ofatumumab and the human placental perfusate, placental perfusate cells, PINK cells, combined NK cells, or combinations thereof are administered intracranially. In yet other embodiments, ofatumumab and the human placental perfusate, placental perfusate cells, PINK cells, combined NK cells, or combinations thereof are administered intrathecally. In still other embodiments, ofatumumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, ofatumumab and the human placental perfusate, placental perfusate cells, PINK cells, combined NK cells, or combinations thereof are delivered via intracranial or intravertebral needles and/or catheters with or without pump devices. In certain embodiments, rituximab and the human placental perfusate, placental perfusate cells, PINK cells, combined NK cells, or combinations thereof are administered concurrently. In other embodiments, rituximab is administered before administration of the human placental perfusate, placental perfusate cells, PINK cells, combined NK cells, or combinations thereof. In yet other embodiments, rituximab is administered after administration of the human placental perfusate, placental perfusate cells, PINK cells, combined NK cells, or combinations thereof. In certain embodiments, rituximab and the human placental perfusate, placental perfusate cells, PINK cells, combined NK cells, or combinations thereof are administered via the same route of administration. In other embodiments, rituximab and the human placental perfusate, placental perfusate cells, PINK cells, combined NK cells, or combinations thereof are administered via different routes of administration. In certain embodiments, rituximab and the human placental perfusate, placental perfusate cells, PINK cells, combined NK cells, or combinations thereof are administered intravenously. In other embodiments, rituximab and the human placental perfusate, placental perfusate cells, PINK cells, combined NK cells, or combinations thereof are administered intracranially. In yet other embodiments, rituximab and the human placental perfusate, placental perfusate cells, PINK cells, combined NK cells, or combinations thereof are administered intrathecally. In still other embodiments, rituximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, rituximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and obinutumumab is administered intracranially to an individual. In some embodiments, the human placental perfusate, human placental perfusate cells, PINK cells, combined NK cells, populations of cells comprising such cells, or combinations thereof, are administered intracranially, and obinutumumab is administered intrathecally to an individual. In other embodiments, the human placental perfusate, human placental perfusate cells, PINK cells, combined NK cells, populations of cells comprising such cells, or combinations thereof, are administered intrathecally, and obinutumumab is administered intravenously to an individual.

In certain embodiments of various methods provided herein, the human placental perfusate, human placental perfusate cells, PINK cells, combined NK cells, populations of cells comprising such cells, or combinations thereof, are administered intravenously, and obinutumumab is administered intrathecally to an individual. In some embodiments, the human placental perfusate, human placental perfusate cells, PINK cells, combined NK cells, populations of cells comprising such cells, or combinations thereof, are administered intracranially, and obinutumumab is administered intravenously to an individual. In other embodiments, the human placental perfusate, human placental perfusate cells, PINK cells, combined NK cells, populations of cells comprising such cells, or combinations thereof, are administered intrathecally, and obinutumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and obinutumumab is administered by an implanted catheter to an individual. In some embodiments, the human placental perfusate, human placental perfusate cells, PINK cells, combined NK cells, populations of cells comprising such cells, or combinations thereof, are administered intracranially, and obinutumumab is administered an implanted catheter to an individual. In other embodiments, the human placental perfusate, human placental perfusate cells, PINK cells, combined NK cells, populations of cells comprising such cells, or combinations thereof, are administered intrathecally, and obinutumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and obinutumumab is administered intrathecally to an individual. In some embodiments, the human placental perfusate, human placental perfusate cells, PINK cells, combined NK cells, populations of cells comprising such cells, or combinations thereof, are administered an implanted catheter, and obinutumumab is administered intravenously to an individual. In other embodiments, the human placental perfusate, human placental perfusate cells, PINK cells, combined NK cells, populations of cells comprising such cells, or combinations thereof, are administered an implanted catheter, and obinutumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and ofatumumab is administered intracranially to an individual. In some embodiments, the human placental perfusate, human placental perfusate cells, PINK cells, combined NK cells, populations of cells comprising such cells, or combinations thereof, are administered intracranially, and ofatumumab is administered intrathecally to an individual. In other embodiments, the human placental perfusate, human placental perfusate cells, PINK cells, combined NK cells, populations of cells comprising such cells, or combinations thereof, are administered intrathecally, and ofatumumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and ofatumumab is administered intrathecally to an individual. In some embodiments, the human placental perfusate, human placental perfusate cells, PINK cells, combined NK cells, populations of cells comprising such cells, or combinations thereof, are administered intracranially, and ofatumumab is administered intravenously to an individual. In other embodiments, the human placental perfusate, human placental perfusate cells, PINK cells, combined NK cells, populations of cells comprising such cells, or combinations thereof, are administered intrathecally, and ofatumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and ofatumumab is administered by an implanted catheter to an individual. In some embodiments, the human placental perfusate, human placental perfusate cells, PINK cells, combined NK cells, populations of cells comprising such cells, or combinations thereof, are administered intracranially, and ofatumumab is administered an implanted catheter to an individual. In other embodiments, the human placental perfusate, human placental perfusate cells, PINK cells, combined NK cells, populations of cells comprising such cells, or combinations thereof, are administered intrathecally, and ofatumumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and ofatumumab is administered intrathecally to an individual. In some embodiments, the human placental perfusate, human placental perfusate cells, PINK cells, combined NK cells, populations of cells comprising such cells, or combinations thereof, are administered an implanted catheter, and ofatumumab is administered intravenously to an individual. In other embodiments, the human placental perfusate, human placental perfusate cells, PINK cells, combined NK cells, populations of cells comprising such cells, or combinations thereof, are administered an implanted catheter, and ofatumumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and rituximab is administered intracranially to an individual. In some embodiments, the human placental perfusate, human placental perfusate cells, PINK cells, combined NK cells, populations of cells comprising such cells, or combinations thereof, are administered intracranially, and rituximab is administered intrathecally to an individual. In other embodiments, the human placental perfusate, human placental perfusate cells, PINK cells, combined NK cells, populations of cells comprising such cells, or combinations thereof, are administered intrathecally, and rituximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and rituximab is administered intrathecally to an individual. In some embodiments, the human placental perfusate, human placental perfusate cells, PINK cells, combined NK cells, populations of cells comprising such cells, or combinations thereof, are administered intracranially, and rituximab is administered intravenously to an individual. In other embodiments, the human placental perfusate, human placental perfusate cells, PINK cells, combined NK cells, populations of cells comprising such cells, or combinations thereof, are administered intrathecally, and rituximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and rituximab is administered by an implanted catheter to an individual. In some embodiments, the human placental perfusate, human placental perfusate cells, PINK cells, combined NK cells, populations of cells comprising such cells, or combinations thereof, are administered intracranially, and rituximab is administered an implanted catheter to an individual. In other embodiments, the human placental perfusate, human placental perfusate cells, PINK cells, combined NK cells, populations of cells comprising such cells, or combinations thereof, are administered intrathecally, and rituximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and rituximab is administered intrathecally to an individual. In some embodiments, the human placental perfusate, human placental perfusate cells, PINK cells, combined NK cells, populations of cells comprising such cells, or combinations thereof, are administered an implanted catheter, and rituximab is administered intravenously to an individual. In other embodiments, the human placental perfusate, human placental perfusate cells, PINK cells, combined NK cells, populations of cells comprising such cells, or combinations thereof, are administered an implanted catheter, and rituximab is administered intracranially to an individual.

In certain embodiments, the anti-GD2 antibody and the human placental perfusate, placental perfusate cells, PINK cells, combined NK cells, or combinations thereof are administered concurrently. In other embodiments, the anti-GD2 antibody is administered before administration of the human placental perfusate, placental perfusate cells, PINK cells, combined NK cells, or combinations thereof. In yet other embodiments, the anti-GD2 antibody is administered after administration of the human placental perfusate, placental perfusate cells, PINK cells, combined NK cells, or combinations thereof. In one embodiment, the anti-GD2 antibody is dinutuximab. In certain embodiments, dinutuximab and the human placental perfusate, placental perfusate cells, PINK cells, combined NK cells, or combinations thereof are administered concurrently. In other embodiments, dinutuximab is administered before administration of the human placental perfusate, placental perfusate cells, PINK cells, combined NK cells, or combinations thereof. In yet other embodiments, dinutuximab is administered after administration of the human placental perfusate, placental perfusate cells, PINK cells, combined NK cells, or combinations thereof. In certain embodiments, dinutuximab and the human placental perfusate, placental perfusate cells, PINK cells, combined NK cells, or combinations thereof are administered via the same route of administration. In other embodiments, dinutuximab and the human placental perfusate, placental perfusate cells, PINK cells, combined NK cells, or combinations thereof are administered via different routes of administration. In certain embodiments, dinutuximab and the human placental perfusate, placental perfusate cells, PINK cells, combined NK cells, or combinations thereof are administered intravenously. In other embodiments, dinutuximab and the human placental perfusate, placental perfusate cells, PINK cells, combined NK cells, or combinations thereof are administered intracranially. In yet other embodiments, dinutuximab and the human placental perfusate, placental perfusate cells, PINK cells, combined NK cells, or combinations thereof are administered intrathecally. In still other embodiments, dinutuximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, dinutuximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and dinutuximab is administered intracranially to an individual. In some embodiments, the human placental perfusate, human placental perfusate cells, PINK cells, combined NK cells, populations of cells comprising such cells, or combinations thereof, are administered intracranially, and dinutuximab is administered intrathecally to an individual. In other embodiments, the human placental perfusate, human placental perfusate cells, PINK cells, combined NK cells, populations of cells comprising such cells, or combinations thereof, are administered intrathecally, and dinutuximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and dinutuximab is administered intrathecally to an individual. In some embodiments, the human placental perfusate, human placental perfusate cells, PINK cells, combined NK cells, populations of cells comprising such cells, or combinations thereof, are administered intracranially, and dinutuximab is administered intravenously to an individual. In other embodiments, the human placental perfusate, human placental perfusate cells, PINK cells, combined NK cells, populations of cells comprising such cells, or combinations thereof, are administered intrathecally, and dinutuximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and dinutuximab is administered by an implanted catheter to an individual. In some embodiments, the human placental perfusate, human placental perfusate cells, PINK cells, combined NK cells, populations of cells comprising such cells, or combinations thereof, are administered intracranially, and dinutuximab is administered an implanted catheter to an individual. In other embodiments, the human placental perfusate, human placental perfusate cells, PINK cells, combined NK cells, populations of cells comprising such cells, or combinations thereof, are administered intrathecally, and dinutuximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and dinutuximab is administered intrathecally to an individual. In some embodiments, the human placental perfusate, human placental perfusate cells, PINK cells, combined NK cells, populations of cells comprising such cells, or combinations thereof, are administered an implanted catheter, and dinutuximab is administered intravenously to an individual. In other embodiments, the human placental perfusate, human placental perfusate cells, PINK cells, combined NK cells, populations of cells comprising such cells, or combinations thereof, are administered an implanted catheter, and dinutuximab is administered intracranially to an individual.

In certain embodiments, the anti-HER2 antibody and the human placental perfusate, placental perfusate cells, PINK cells, combined NK cells, or combinations thereof are administered concurrently. In other embodiments, the anti-HER2 antibody is administered before administration of the human placental perfusate, placental perfusate cells, PINK cells, combined NK cells, or combinations thereof. In yet other embodiments, the anti-HER2 antibody is administered after administration of the human placental perfusate, placental perfusate cells, PINK cells, combined NK cells, or combinations thereof. In one embodiment, the anti-HER2 antibody is trastuzumab. In certain embodiments, trastuzumab and the human placental perfusate, placental perfusate cells, PINK cells, combined NK cells, or combinations thereof are administered concurrently. In other embodiments, trastuzumab is administered before administration of the human placental perfusate, placental perfusate cells, PINK cells, combined NK cells, or combinations thereof. In yet other embodiments, trastuzumab is administered after administration of the human placental perfusate, placental perfusate cells, PINK cells, combined NK cells, or combinations thereof. In certain embodiments, trastuzumab and the human placental perfusate, placental perfusate cells, PINK cells, combined NK cells, or combinations thereof are administered via the same route of administration. In other embodiments, trastuzumab and the human placental perfusate, placental perfusate cells, PINK cells, combined NK cells, or combinations thereof are administered via different routes of administration. In certain embodiments, trastuzumab and the human placental perfusate, placental perfusate cells, PINK cells, combined NK cells, or combinations thereof are administered intravenously. In other embodiments, trastuzumab and the human placental perfusate, placental perfusate cells, PINK cells, combined NK cells, or combinations thereof are administered intracranially. In yet other embodiments, trastuzumab and the human placental perfusate, placental perfusate cells, PINK cells, combined NK cells, or combinations thereof are administered intrathecally. In still other embodiments, trastuzumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, trastuzumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and trastuzumab is administered intracranially to an individual. In some embodiments, the human placental perfusate, human placental perfusate cells, PINK cells, combined NK cells, populations of cells comprising such cells, or combinations thereof, are administered intracranially, and trastuzumab is administered intrathecally to an individual. In other embodiments, the human placental perfusate, human placental perfusate cells, PINK cells, combined NK cells, populations of cells comprising such cells, or combinations thereof, are administered intrathecally, and trastuzumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and trastuzumab is administered intrathecally to an individual. In some embodiments, the human placental perfusate, human placental perfusate cells, PINK cells, combined NK cells, populations of cells comprising such cells, or combinations thereof, are administered intracranially, and trastuzumab is administered intravenously to an individual. In other embodiments, the human placental perfusate, human placental perfusate cells, PINK cells, combined NK cells, populations of cells comprising such cells, or combinations thereof, are administered intrathecally, and trastuzumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and trastuzumab is administered by an implanted catheter to an individual. In some embodiments, the human placental perfusate, human placental perfusate cells, PINK cells, combined NK cells, populations of cells comprising such cells, or combinations thereof, are administered intracranially, and trastuzumab is administered an implanted catheter to an individual. In other embodiments, the human placental perfusate, human placental perfusate cells, PINK cells, combined NK cells, populations of cells comprising such cells, or combinations thereof, are administered intrathecally, and trastuzumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and trastuzumab is administered intrathecally to an individual. In some embodiments, the human placental perfusate, human placental perfusate cells, PINK cells, combined NK cells, populations of cells comprising such cells, or combinations thereof, are administered an implanted catheter, and trastuzumab is administered intravenously to an individual. In other embodiments, the human placental perfusate, human placental perfusate cells, PINK cells, combined NK cells, populations of cells comprising such cells, or combinations thereof, are administered an implanted catheter, and trastuzumab is administered intracranially to an individual.

In certain embodiments, the anti-PD-L1 antibody and the human placental perfusate, placental perfusate cells, PINK cells, combined NK cells, or combinations thereof are administered concurrently. In other embodiments, the anti-PD-L1 antibody is administered before administration of the human placental perfusate, placental perfusate cells, PINK cells, combined NK cells, or combinations thereof. In yet other embodiments, the anti-PD-L1 antibody is administered after administration of the human placental perfusate, placental perfusate cells, PINK cells, combined NK cells, or combinations thereof. In one embodiment, the anti-PD-L1 antibody is atezolizumab. In another embodiment, the anti-PD-L1 antibody is avelumab. In certain embodiments, atezolizumab and the human placental perfusate, placental perfusate cells, PINK cells, combined NK cells, or combinations thereof are administered concurrently. In other embodiments, atezolizumab is administered before administration of the human placental perfusate, placental perfusate cells, PINK cells, combined NK cells, or combinations thereof. In yet other embodiments, atezolizumab is administered after administration of the human placental perfusate, placental perfusate cells, PINK cells, combined NK cells, or combinations thereof. In certain embodiments, atezolizumab and the human placental perfusate, placental perfusate cells, PINK cells, combined NK cells, or combinations thereof are administered via the same route of administration. In other embodiments, atezolizumab and the human placental perfusate, placental perfusate cells, PINK cells, combined NK cells, or combinations thereof are administered via different routes of administration. In certain embodiments, atezolizumab and the human placental perfusate, placental perfusate cells, PINK cells, combined NK cells, or combinations thereof are administered intravenously. In other embodiments, atezolizumab and the human placental perfusate, placental perfusate cells, PINK cells, combined NK cells, or combinations thereof are administered intracranially. In yet other embodiments, atezolizumab and the human placental perfusate, placental perfusate cells, PINK cells, combined NK cells, or combinations thereof are administered intrathecally. In still other embodiments, atezolizumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, atezolizumab and the human placental perfusate, placental perfusate cells, PINK cells, combined NK cells, or combinations thereof are delivered via intracranial or intravertebral needles and/or catheters with or without pump devices. In certain embodiments, avelumab and the human placental perfusate, placental perfusate cells, PINK cells, combined NK cells, or combinations thereof are administered concurrently. In other embodiments, avelumab is administered before administration of the human placental perfusate, placental perfusate cells, PINK cells, combined NK cells, or combinations thereof. In yet other embodiments, avelumab is administered after administration of the human placental perfusate, placental perfusate cells, PINK cells, combined NK cells, or combinations thereof. In certain embodiments, avelumab and the human placental perfusate, placental perfusate cells, PINK cells, combined NK cells, or combinations thereof are administered via the same route of administration. In other embodiments, avelumab and the human placental perfusate, placental perfusate cells, PINK cells, combined NK cells, or combinations thereof are administered via different routes of administration. In certain embodiments, avelumab and the human placental perfusate, placental perfusate cells, PINK cells, combined NK cells, or combinations thereof are administered intravenously. In other embodiments, avelumab and the human placental perfusate, placental perfusate cells, PINK cells, combined NK cells, or combinations thereof are administered intracranially. In yet other embodiments, avelumab and the human placental perfusate, placental perfusate cells, PINK cells, combined NK cells, or combinations thereof are administered intrathecally. In still other embodiments, avelumab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, avelumab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and atezolizumab is administered intracranially to an individual. In some embodiments, the human placental perfusate, human placental perfusate cells, PINK cells, combined NK cells, populations of cells comprising such cells, or combinations thereof, are administered intracranially, and atezolizumab is administered intrathecally to an individual. In other embodiments, the human placental perfusate, human placental perfusate cells, PINK cells, combined NK cells, populations of cells comprising such cells, or combinations thereof, are administered intrathecally, and atezolizumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and atezolizumab is administered intrathecally to an individual. In some embodiments, the human placental perfusate, human placental perfusate cells, PINK cells, combined NK cells, populations of cells comprising such cells, or combinations thereof, are administered intracranially, and atezolizumab is administered intravenously to an individual. In other embodiments, the human placental perfusate, human placental perfusate cells, PINK cells, combined NK cells, populations of cells comprising such cells, or combinations thereof, are administered intrathecally, and atezolizumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and atezolizumab is administered by an implanted catheter to an individual. In some embodiments, the human placental perfusate, human placental perfusate cells, PINK cells, combined NK cells, populations of cells comprising such cells, or combinations thereof, are administered intracranially, and atezolizumab is administered an implanted catheter to an individual. In other embodiments, the human placental perfusate, human placental perfusate cells, PINK cells, combined NK cells, populations of cells comprising such cells, or combinations thereof, are administered intrathecally, and atezolizumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and atezolizumab is administered intrathecally to an individual. In some embodiments, the human placental perfusate, human placental perfusate cells, PINK cells, combined NK cells, populations of cells comprising such cells, or combinations thereof, are administered an implanted catheter, and atezolizumab is administered intravenously to an individual. In other embodiments, the human placental perfusate, human placental perfusate cells, PINK cells, combined NK cells, populations of cells comprising such cells, or combinations thereof, are administered an implanted catheter, and atezolizumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and avelumab is administered intracranially to an individual. In some embodiments, the human placental perfusate, human placental perfusate cells, PINK cells, combined NK cells, populations of cells comprising such cells, or combinations thereof, are administered intracranially, and avelumab is administered intrathecally to an individual. In other embodiments, the human placental perfusate, human placental perfusate cells, PINK cells, combined NK cells, populations of cells comprising such cells, or combinations thereof, are administered intrathecally, and avelumab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and avelumab is administered intrathecally to an individual. In some embodiments, the human placental perfusate, human placental perfusate cells, PINK cells, combined NK cells, populations of cells comprising such cells, or combinations thereof, are administered intracranially, and avelumab is administered intravenously to an individual. In other embodiments, the human placental perfusate, human placental perfusate cells, PINK cells, combined NK cells, populations of cells comprising such cells, or combinations thereof, are administered intrathecally, and avelumab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and avelumab is administered by an implanted catheter to an individual. In some embodiments, the human placental perfusate, human placental perfusate cells, PINK cells, combined NK cells, populations of cells comprising such cells, or combinations thereof, are administered intracranially, and avelumab is administered an implanted catheter to an individual. In other embodiments, the human placental perfusate, human placental perfusate cells, PINK cells, combined NK cells, populations of cells comprising such cells, or combinations thereof, are administered intrathecally, and avelumab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and avelumab is administered intrathecally to an individual. In some embodiments, the human placental perfusate, human placental perfusate cells, PINK cells, combined NK cells, populations of cells comprising such cells, or combinations thereof, are administered an implanted catheter, and avelumab is administered intravenously to an individual. In other embodiments, the human placental perfusate, human placental perfusate cells, PINK cells, combined NK cells, populations of cells comprising such cells, or combinations thereof, are administered an implanted catheter, and avelumab is administered intracranially to an individual.

In certain embodiments, the anti-EGFR antibody and the human placental perfusate, placental perfusate cells, PINK cells, combined NK cells, or combinations thereof are administered concurrently. In other embodiments, the anti-EGFR antibody is administered before administration of the human placental perfusate, placental perfusate cells, PINK cells, combined NK cells, or combinations thereof. In yet other embodiments, the anti-EGFR antibody is administered after administration of the human placental perfusate, placental perfusate cells, PINK cells, combined NK cells, or combinations thereof. In one embodiment, the anti-EGFR antibody is cetuximab. In certain embodiments, cetuximab and the human placental perfusate, placental perfusate cells, PINK cells, combined NK cells, or combinations thereof are administered concurrently. In other embodiments, cetuximab is administered before administration of the human placental perfusate, placental perfusate cells, PINK cells, combined NK cells, or combinations thereof. In yet other embodiments, cetuximab is administered after administration of the human placental perfusate, placental perfusate cells, PINK cells, combined NK cells, or combinations thereof. In certain embodiments, cetuximab and the human placental perfusate, placental perfusate cells, PINK cells, combined NK cells, or combinations thereof are administered via the same route of administration. In other embodiments, cetuximab and the human placental perfusate, placental perfusate cells, PINK cells, combined NK cells, or combinations thereof are administered via different routes of administration. In certain embodiments, cetuximab and the human placental perfusate, placental perfusate cells, PINK cells, combined NK cells, or combinations thereof are administered intravenously. In other embodiments, cetuximab and the human placental perfusate, placental perfusate cells, PINK cells, combined NK cells, or combinations thereof are administered intracranially. In yet other embodiments, cetuximab and the human placental perfusate, placental perfusate cells, PINK cells, combined NK cells, or combinations thereof are administered intrathecally. In still other embodiments, cetuximab and the human placental perfusate, placental perfusate cells, PINK cells, combined NK cells, or combinations thereof are surgically implanted, injected, infused, e.g., by way of a catheter or syringe, or otherwise administered directly or indirectly to the site in need of repair or augmentation. In still other embodiments, cetuximab and the human placental perfusate, placental perfusate cells, PINK cells, combined NK cells, or combinations thereof are delivered via intracranial or intravertebral needles and/or catheters with or without pump devices.

In certain embodiments of various methods provided herein, the human placental perfusate, human placental perfusate cells, PINK cells, combined NK cells, populations of cells comprising such cells, or combinations thereof, are administered intravenously, and cetuximab is administered intracranially to an individual. In some embodiments, the human placental perfusate, human placental perfusate cells, PINK cells, combined NK cells, populations of cells comprising such cells, or combinations thereof, are administered intracranially, and cetuximab is administered intrathecally to an individual. In other embodiments, the human placental perfusate, human placental perfusate cells, PINK cells, combined NK cells, populations of cells comprising such cells, or combinations thereof, are administered intrathecally, and cetuximab is administered intravenously to an individual. In certain embodiments of various methods provided herein, the human placental perfusate, human placental perfusate cells, PINK cells, combined NK cells, populations of cells comprising such cells, or combinations thereof, are administered intravenously, and cetuximab is administered intrathecally to an individual. In some embodiments, the human placental perfusate, human placental perfusate cells, PINK cells, combined NK cells, populations of cells comprising such cells, or combinations thereof, are administered intracranially, and cetuximab is administered intravenously to an individual. In other embodiments, the human placental perfusate, human placental perfusate cells, PINK cells, combined NK cells, populations of cells comprising such cells, or combinations thereof, are administered intrathecally, and cetuximab is administered intracranially to an individual.

In certain embodiments of various methods provided herein, the human placental perfusate, human placental perfusate cells, PINK cells, combined NK cells, populations of cells comprising such cells, or combinations thereof, are administered intravenously, and cetuximab is administered by an implanted catheter to an individual. In some embodiments, the human placental perfusate, human placental perfusate cells, PINK cells, combined NK cells, populations of cells comprising such cells, or combinations thereof, are administered intracranially, and cetuximab is administered an implanted catheter to an individual. In other embodiments, the human placental perfusate, human placental perfusate cells, PINK cells, combined NK cells, populations of cells comprising such cells, or combinations thereof, are administered intrathecally, and cetuximab is administered an implanted catheter to an individual. In certain embodiments of various methods provided herein, the human placental perfusate, human placental perfusate cells, PINK cells, combined NK cells, populations of cells comprising such cells, or combinations thereof, are administered an implanted catheter, and cetuximab is administered intrathecally to an individual. In some embodiments, the human placental perfusate, human placental perfusate cells, PINK cells, combined NK cells, populations of cells comprising such cells, or combinations thereof, are administered an implanted catheter, and cetuximab is administered intravenously to an individual. In other embodiments, the human placental perfusate, human placental perfusate cells, PINK cells, combined NK cells, populations of cells comprising such cells, or combinations thereof, are administered an implanted catheter, and cetuximab is administered intracranially to an individual.

The human placental perfusate, human placental perfusate cells, PINK cells, combined NK cells, populations of cells comprising such cells, or combinations thereof, and the antibody can be administered to an individual in a composition, e.g., a matrix, hydrogel, scaffold, or the like that comprise the cells and the antibody.

In one embodiment, the cells provided herein are seeded onto a natural matrix, e.g., a placental biomaterial such as an amniotic membrane material. Such an amniotic membrane material can be, e.g., amniotic membrane dissected directly from a mammalian placenta; fixed or heat-treated amniotic membrane, substantially dry (i.e., <20% H₂O) amniotic membrane, chorionic membrane, substantially dry chorionic membrane, substantially dry amniotic and chorionic membrane, and the like. Preferred placental biomaterials on which placental stem cells can be seeded are described in Hariri, U.S. Application Publication No. 2004/0048796, the disclosure of which is hereby incorporated by reference in its entirety.

In another embodiment, the human placental perfusate, human placental perfusate cells, PINK cells, combined NK cells, populations of cells comprising such cells, or combinations thereof, and the antibody are suspended in a hydrogel solution suitable for, e.g., injection. Suitable hydrogels for such compositions include self-assembling peptides, such as RAD16. In one embodiment, a hydrogel solution comprising the cells and the antibody can be allowed to harden, for instance in a mold, to form a matrix having cells and antibody dispersed therein for implantation. The cells in such a matrix can also be cultured so that the cells are mitotically expanded prior to implantation. The hydrogel can be, for example, an organic polymer (natural or synthetic) that is cross-linked via covalent, ionic, or hydrogen bonds to create a three-dimensional open-lattice structure that entraps water molecules to form a gel. Hydrogel-forming materials include polysaccharides such as alginate and salts thereof, peptides, polyphosphazines, and polyacrylates, which are crosslinked ionically, or block polymers such as polyethylene oxide-polypropylene glycol block copolymers which are crosslinked by temperature or pH, respectively. In some embodiments, the hydrogel or matrix of the invention is biodegradable.

In some embodiments of the invention, the formulation comprises an in situ polymerizable gel (see., e.g., U.S. Patent Application Publication 2002/0022676; Anseth et al., J. Control Release, 78(1-3):199-209 (2002); Wang et al., Biomaterials, 24(22):3969-80 (2003).

In some embodiments, the polymers are at least partially soluble in aqueous solutions, such as water, buffered salt solutions, or aqueous alcohol solutions, that have charged side groups, or a monovalent ionic salt thereof. Examples of polymers having acidic side groups that can be reacted with cations are poly(phosphazenes), poly(acrylic acids), poly(methacrylic acids), copolymers of acrylic acid and methacrylic acid, poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups, sulfonic acid groups, halogenated (preferably fluorinated) alcohol groups, phenolic OH groups, and acidic OH groups.

The human placental perfusate cells, PINK cells, combined NK cells, populations of cells comprising such cells, or combinations thereof, can be seeded onto a three-dimensional framework or scaffold and implanted in vivo. Such a framework can be implanted in combination with any one or more growth factors, cells, drugs or other components that stimulate tissue formation or otherwise enhance or improve the practice of the invention.

Examples of scaffolds that can be used in the present invention include nonwoven mats, porous foams, or self assembling peptides. Nonwoven mats can be formed using fibers comprised of a synthetic absorbable copolymer of glycolic and lactic acids (e.g., PGA/PLA) (VICRYL, Ethicon, Inc., Somerville, N.J.). Foams, composed of, e.g., poly(ε-caprolactone)/poly(glycolic acid) (PCL/PGA) copolymer, formed by processes such as freeze-drying, or lyophilization (see, e.g., U.S. Pat. No. 6,355,699), can also be used as scaffolds.

The human placental perfusate cells, PINK cells, combined NK cells, populations of cells comprising such cells, or combinations thereof, can also be seeded onto, or contacted with, a physiologically-acceptable ceramic material including, but not limited to, mono-, di-, tri-, alpha-tri-, beta-tri-, and tetra-calcium phosphate, hydroxyapatite, fluoroapatites, calcium sulfates, calcium fluorides, calcium oxides, calcium carbonates, magnesium calcium phosphates, biologically active glasses such as BIOGLASS®, and mixtures thereof. Porous biocompatible ceramic materials currently commercially available include SURGIBONE® (CanMedica Corp., Canada), ENDOBON® (Merck Biomaterial France, France), CEROS® (Mathys, AG, Bettlach, Switzerland), and mineralized collagen bone grafting products such as HEALOS™ (DePuy, Inc., Raynham, Mass.) and VITOSS®, RHAKOSS™, and CORTOSS® (Orthovita, Malvem, Pa.). The framework can be a mixture, blend or composite of natural and/or synthetic materials.

In another embodiment, the human placental perfusate cells, PINK cells, combined NK cells, populations of cells comprising such cells, or combinations thereof, can be seeded onto, or contacted with, a felt, which can be, e.g., composed of a multifilament yarn made from a bioabsorbable material such as PGA, PLA, PCL copolymers or blends, or hyaluronic acid.

The human placental perfusate cells, PINK cells, combined NK cells, populations of cells comprising such cells, or combinations thereof, can, in another embodiment, be seeded onto foam scaffolds that may be composite structures. Such foam scaffolds can be molded into a useful shape, such as that of a portion of a specific structure in the body to be repaired, replaced or augmented. In some embodiments, the framework is treated, e.g., with 0.1 M acetic acid followed by incubation in polylysine, PBS, and/or collagen, prior to inoculation of the cells in order to enhance cell attachment. External surfaces of a matrix may be modified to improve the attachment or growth of cells and differentiation of tissue, such as by plasma-coating the matrix, or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate, etc.), a cellular matrix, and/or other materials such as, but not limited to, gelatin, alginates, agar, agarose, and plant gums, and the like.

In some embodiments, the scaffold comprises, or is treated with, materials that render it non-thrombogenic. These treatments and materials may also promote and sustain endothelial growth, migration, and extracellular matrix deposition. Examples of these materials and treatments include but are not limited to natural materials such as basement membrane proteins such as laminin and Type IV collagen, synthetic materials such as EPTFE, and segmented polyurethaneurea silicones, such as PURSPAN™ (The Polymer Technology Group, Inc., Berkeley, Calif.). The scaffold can also comprise anti-thrombotic agents such as heparin; the scaffolds can also be treated to alter the surface charge (e.g., coating with plasma) prior to seeding with placental stem cells.

### 5.8. Compositions Comprising Human Placental Perfusate, Placental Perfusate Cells, PINK Cells, Combined NK Cells, or Combinations Thereof and Methods of Use Thereof

In another aspect, provided herein are compositions comprising human placental perfusate, placental perfusate cells, PINK cells, combined NK cells, or combinations thereof, in combination with an antibody. Any combinations of human placental perfusate, placental perfusate cells, PINK cells, combined NK cells, or combinations thereof, described herein are contemplated. In certain embodiments, the composition comprises more than one antibody. In some embodiments, the composition comprises two, three, four, or five antibodies. In other embodiments, the composition comprises human placental perfusate, placental perfusate cells, PINK cells, combined NK cells, or combinations thereof, and an antibody, and further comprises one or more additional agents. In yet other embodiments, the additional agents are anti-cancer agents selected from the list described in section 5.7. In still other embodiments, the additional agents are excipients, buffers, stabilizers, media, media supplements, antibiotics, or any additives known in the art that help maintain the stability and/or activity of the compositions.

In another aspect, provided herein are methods of suppressing growth or proliferation of tumor cells by contacting the tumor cells with the composition that comprises human placental perfusate, placental perfusate cells, PINK cells, combined NK cells, or combinations thereof, in combination with an antibody, as described herein. Also provided herein are methods of treating an individual having tumor cells by administering a therapeutically effective amount of the composition that comprises human placental perfusate, placental perfusate cells, PINK cells, combined NK cells, or combinations thereof, in combination with an antibody, as described herein, to the individual.

In a specific embodiment, the tumor cells are blood cancer cells. In another embodiment, the tumor cells are solid tumor cells. In various specific embodiments, the tumor cells are glioblastoma cells, neuroblastoma cells, osteosarcoma cells, melanoma cells, ovarian cancer cells, primary ductal carcinoma cells, leukemia cells, acute T cell leukemia cells, AML cells, CML cells, ALL cells, CLL cells, NHL cells, breast cancer cells, bladder cancer cells, Merkel cell carcinoma cells, head and neck cancer cells, lung carcinoma cells, colon adenocarcinoma cells, histiocytic lymphoma cells, multiple myeloma cells, retinoblastoma cells, colorectal carcinoma cells, colorectal adenocarcinoma cells. In one embodiment, the tumor cells are glioblastoma cells. In one embodiment, the tumor cells are neuroblastoma cells. In one embodiment, the tumor cells are osteosarcoma cells. In one embodiment, the tumor cells are melanoma cells. In one embodiment, the tumor cells are ovarian cancer cells. In one embodiment, the tumor cells are primary ductal carcinoma cells. In one embodiment, the tumor cells are leukemia cells. In one embodiment, the tumor cells are acute T cell leukemia cells. In one embodiment, the tumor cells are AML cells. In one embodiment, the tumor cells are CML cells. In one embodiment, the tumor cells are ALL cells. In one embodiment, the tumor cells are CLL cells. In one embodiment, the tumor cells are NHL cells. In one embodiment, the tumor cells are breast cancer cells. In one embodiment, the tumor cells are bladder cancer cells. In one embodiment, the tumor cells are Merkel cell carcinoma cells. In one embodiment, the tumor cells are head and neck cancer cells. In one embodiment, the tumor cells are lung carcinoma cells. In one embodiment, the tumor cells are colon adenocarcinoma cells. In one embodiment, the tumor cells are histiocytic lymphoma cells. In one embodiment, the tumor cells are multiple myeloma cells. In one embodiment, the tumor cells are retinoblastoma cells. In one embodiment, the tumor cells are colorectal carcinoma cells. In one embodiment, the tumor cells are colorectal adenocarcinoma cells.

The treatment with the natural killer cells of the invention can be part of an anticancer therapy regimen that further includes one or more other anticancer agents. Such anticancer agents are well-known in the art. Specific anticancer agents that may be administered to an individual having cancer, in addition to the human placental perfusate, placental perfusate cells, PINK cells, combined NK cells, pools and/or combinations of the same, include, but are not limited to: acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

In some embodiments of various methods provided herein, the combination therapy of an antibody with human placental perfusate, placental perfusate cells, PINK cells, combined NK cells, or combinations thereof, further comprises a third agent. In certain embodiments, the third agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid.

In other embodiments, the combination therapy of an antibody with human placental perfusate, placental perfusate cells, PINK cells, combined NK cells, or combinations thereof, further comprises a third agent and a fourth agent. In certain embodiments, the third or the fourth agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid. In one embodiment, the third agent is IL-2, and the fourth agent is GM-CSF. In another embodiment, the third agent is IL-2, and the fourth agent is 13-cis retinoic acid. In yet another embodiment, the third agent is GM-CSF, and the fourth agent is 13-cis retinoic acid. In one embodiment, the antibody is an anti-GD2 antibody, the third agent is IL-2, and the fourth agent is GM-CSF. In another embodiment, the antibody is an anti-GD2 antibody, the third agent is IL-2, and the fourth agent is 13-cis retinoic acid. In yet another embodiment, the antibody is an anti-GD2 antibody, the third agent is GM-CSF, and the fourth agent is 13-cis retinoic acid.

In other embodiments, the combination therapy of an antibody with human placental perfusate, placental perfusate cells, PINK cells, combined NK cells, or combinations thereof, further comprises a third agent, a fourth agent, and a fifth agent. In certain embodiments, the third, the fourth, or the fifth agent is a hematopoietic growth factor, cytokine, anti-cancer agent (e.g., a checkpoint inhibitor), antibiotic, cox-2 inhibitor, immunomodulatory agent, immunosuppressive agent, corticosteroid, or derivative thereof. In one embodiment, the third agent is IL-2. In another embodiment, the third agent is GM-CSF. In yet another embodiment, the third agent is 13-cis retinoic acid. In a specific embodiment, the antibody is an anti-GD2 antibody, and the third agent is IL-2. In another specific embodiment, the antibody is an anti-GD2 antibody, and the third agent is GM-CSF. In yet another specific embodiment, the antibody is an anti-GD2 antibody, and the third agent is 13-cis retinoic acid. In one embodiment, the third agent is IL-2, and the fourth agent is GM-CSF. In another embodiment, the third agent is IL-2, and the fourth agent is 13-cis retinoic acid. In yet another embodiment, the third agent is GM-CSF, and the fourth agent is 13-cis retinoic acid. In one embodiment, the antibody is an anti-GD2 antibody, the third agent is IL-2, and the fourth agent is GM-CSF. In another embodiment, the antibody is an anti-GD2 antibody, the third agent is IL-2, and the fourth agent is 13-cis retinoic acid. In yet another embodiment, the antibody is an anti-GD2 antibody, the third agent is GM-CSF, and the fourth agent is 13-cis retinoic acid. In still another embodiment, the third agent is IL-2, the fourth agent is GM-CSF, and the fifth agent is 13-cis retinoic acid. In still another embodiment, the antibody is an anti-GD2 antibody, the third agent is IL-2, the fourth agent is GM-CSF, and the fifth agent is 13-cis retinoic acid.

### 6. EXAMPLES

### 6.1. Example 1: Preparation of PNK Cells

PINK cells, PNK, were generated using the two-step expansion and differentiation method described in U.S. Pat. No. 8,926,964 then frozen. Prior to the cytotoxicity experiments below, PNK cells were thawed, recovered with medium for 3 days, and resuspended at 1×10⁶ cells/mL with assay medium (RPMI 1640 medium supplemented with 10% fetal bovine serum (FBS) and antibiotics (1% penicillin and streptomycin)) for cytotoxicity assay.

### 6.2. Example 2: In Vitro Cytolytic Activity of PNK Cells Against Glioblastoma (GBM) Cell Lines

Tumor cell lines as indicated were labeled with 7.5 M PKH26 fluorescent dye (Sigma-Aldrich, St Louis, Mo., Cat# PKH26-GL). Four-hour cytotoxicity assay was performed by using PNK cells as effector cells and PKH26 labeled tumor cell lines as target cells. Target cell number was fixed at 1×104 while PNK cells were used in different amounts to achieve various E:T ratios (100:1, 20:1, 10:1, 5:1, 2.5:1, 1:1, and 0.6:1) as indicated. In several studies, unrelated healthy donor peripheral blood mononuclear cells (PBMCs) were also included as targets cells as controls. Target cells were incubated with PNK cells in 96-well flat-bottom tissue culture plates in 200 μL of the assay buffer (RPMI 1640 media supplemented with 10% fetal bovine serum (FBS) and antibiotics (1% penicillin and streptomycin)) for 4 hours at 37° C. in 5% CO2. After incubation, cells were harvested and TO-PRO-3 (Invitrogen, Carlsbad, Calif. Cat# T3605), a membrane-impermeable DNA stain, was added to the cultures at 1 μM final concentration in order to identify dead cells (TO-PRO-3+). To determine spontaneous target cell death, PKH26-labeled target cells were cultured alone for the duration of the assay. As a positive control for dead cells, 1×105 labeled target cells were permeabilized with 300 μL of Cytofix/Cytoperm buffer (BD Biosciences, Cat#554722) for 20 minutes at 4° C. Data were acquired on a FACSCanto II (BD Biosciences, San Jose, Calif.) and analyzed in FlowJo (Tree Star, Ashland, Oreg.).

The percentage of dead target cells in each sample was calculated as follows: % TO-PRO-3+PKH26+ cells (Q2)/(% TO-PRO-3+PKH26+(Q2)+% TO-PRO-3-PKH26+(Q1))*100%. Percent cytotoxicity reported was calculated by subtracting the percent of dead target cells in cultures of target cells alone from the percent of dead target cells in co-cultures of PNK and target cells. Results from different experiments were reported as mean±standard deviation of the mean.

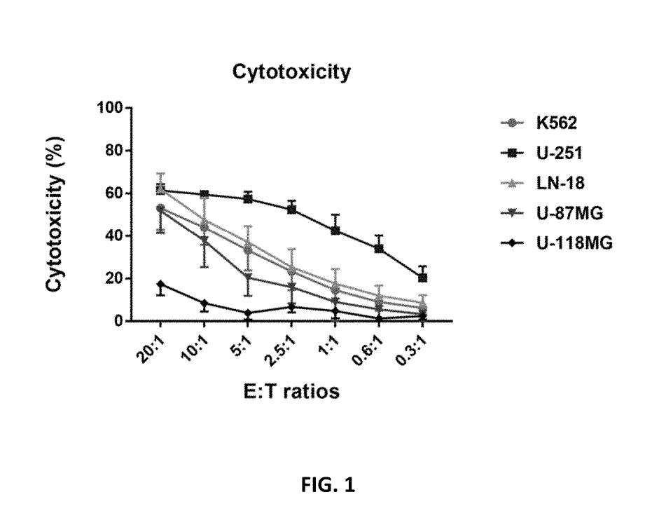

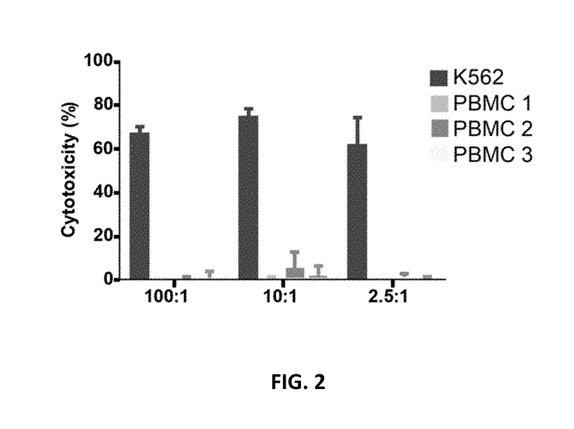

The results showed that PNK displayed cytotoxic activity at an E:T ratio as low as 0.3:1, and that this activity increased with increasing E:T ratios in a dose-dependent manner (FIG. 1). At an E:T ratio of 10:1, PNK cells lysed 59.4%+1.5% of U-251, 47.6%+10.5% of LN-18, 37.7%+12.3% of U-87MG, 8.5%+3.9% of U-118MG respectively within 4 hours of exposure (FIG. 1). Thus, PNK cells displayed cytolytic activity against GBM cell lines. Noted compared with cytotoxicity against K562, higher cytotoxicity against U-251 and comparable cytotoxicity against LN-18 was observed. Furthermore, PNK cells did not show any cytotoxic activity against PBMCs from unrelated healthy donors at any of the E:T ratios tested up to 100:1 (FIG. 2), indicating that PNK cells were capable not only of lysing tumor cells but also of discriminating between healthy and tumor targets.

### 6.3. Example 3: Secretion of IFN-γ, TNF-α and GM-CSF Cytokines of PNK Cells in Response to GBM Tumor Cell Lines

PNK cells were incubated with tumor targets: U-251 or U-87MG in 96-well flat-bottom tissue culture plates at an E:T ratio of 1:1 (1×10⁵ cells each) in 200 μL of RPMI 1640 supplemented with 10% FBS and antibiotics. After 24-hour incubation at 37° C. and 5% CO₂, the supernatant was collected and cytokine concentrations were determined by Luminex analysis using MILLIPLEX MAP magnetic bead kits (EMD Millipore, Billerica, Mass., Cat#HCD8MAG-15K-07 for GM-CSF, perforin, TNF-α, IL-10, granzyme A, granzyme B and IFN-γ; Cat# HCYTOMAG-60K-02 for MCP-1 and IFN-α2) according to the protocol provided by the manufacturer. Data were analyzed using Milliplex Xponent and Analyst software (EMD Millipore).

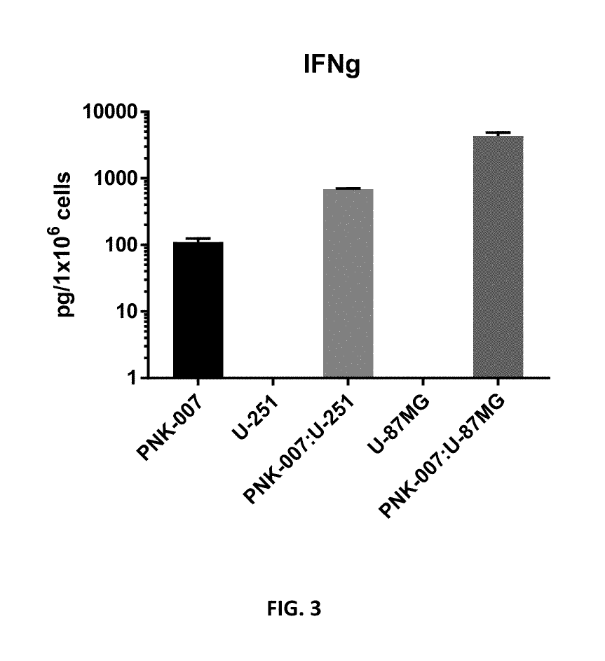

As seen in FIG. 3, PNK cells were capable of secreting IFN-γ in response to U-251 and U-87MG cell lines. Significant difference was shown from PNK cells cocultured with U-251 or U-87MG in comparison with that of PNK alone (P<0.05). A similar pattern of cytokine secretion was observed for TNF-α and GM-CSF from PNK cells cocultured with GBM tumor cell lines.

### 6.4. Example 4: In Vitro Anti-Tumor Activity of PNK Cells in with or without Anti-GD2 Antibody Unituxin® (Dinutuximab)

1×10⁷ cells from each solid tumor cell line were first labeled with 7.5 μM PKH26 fluorescent dye (Sigma-Aldrich, St Louis, Mo., Cat# PKH26-GL), then treated with Unituxin® (dinutuximab, from United Therapeutics, Silver Spring, Md.) at different concentration of 0, 0.01, 0.1, 1, 3, 10, 30, or 100 μg/mL at 37° C. for 30 min. Human IgG1 (BioLegend, Cat#403502) at corresponding concentrations was used as isotype control. The treated cells were washed twice with assay medium (see section 5.1). Four-hour cytotoxicity assay was performed by using PNK cells as effector cells and PKH26 labeled, Unituxin® or human IgG1 treated tumor cells as target cells. Target cell number was fixed at 1×10⁴, while the number of PNK cells was determined based on different effector to target (E:T) ratios, such as 10:1, 3:1, and 1:1. Target cells were incubated with PNK cells in 96-well flat-bottom tissue culture plates in 200 μL of the assay medium for 4 hours at 37° C. in 5% CO₂. After incubation, cells were harvested and TO-PRO-3 (Invitrogen, Carlsbad, Calif., Cat#T3605), a membrane-impermeable DNA stain, was added to the cultures at 1 μM final concentration to identify dead cells (TO-PRO-3+). To determine spontaneous target cell death, PKH26-labeled target cells were cultured alone for the duration of the assay. As a positive control for dead cells, 1×10⁵ labeled target cells were permeabilized with 300 μL of Cytofix/Cytoperm buffer (BD Biosciences, San Jose, Calif., Cat#554722) for 20 minutes at 4° C. Data were acquired on FACSCanto II (BD Biosciences) and analyzed using FlowJo software (Tree Star, Ashland, Oreg.).

The percentage of dead target cells in each sample was calculated as follows: % TO-PRO-3+PKH26⁺ cells/(% TO-PRO-3+PKH26++% TO-PRO-3-PKH26⁺)*100%. The percentage of cytotoxicity was calculated by subtracting the percentage of dead target cells in cultures of the target cells alone from the percentage of dead target cells in co-cultures of PNK cells and the target cells. Results from different experiments were reported as mean±standard deviation of the mean. Two-way analysis of variance was used to assess if there is any interaction between PNK cells and Unituxin®.

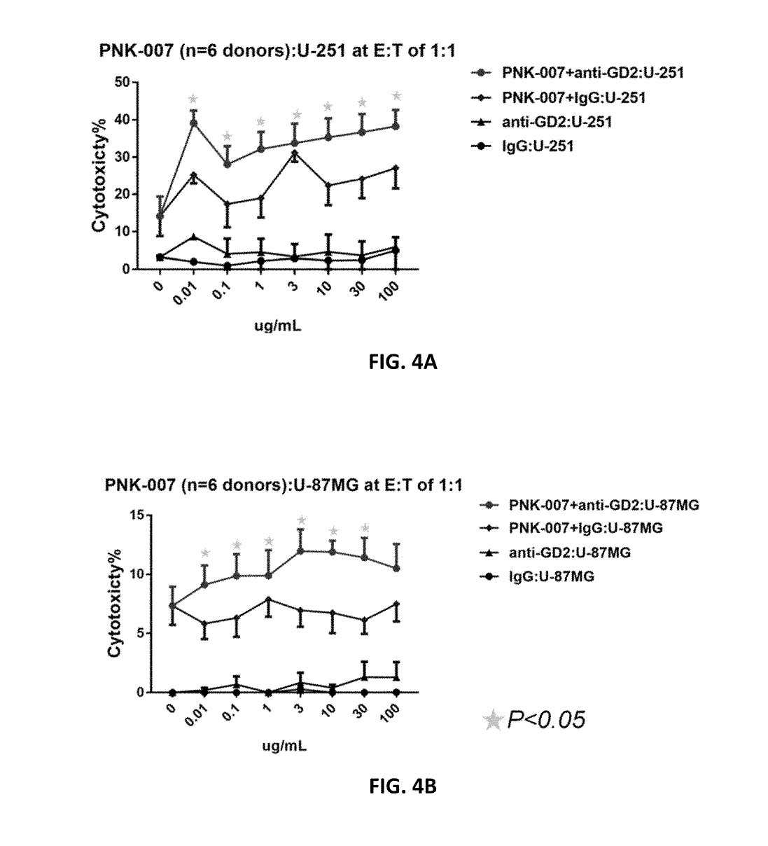

The cytotoxicity of PNK cells against human glioblastoma cell line U-251 significantly increased in presence of Unituxin®, compared with that in presence of IgG1 control, at E:T ratio of 1:1 (FIG. 4A). Similarly, the cytotoxicity of PNK cells against another human glioblastoma cell line U-87MG also was increased significantly in presence of Unituxin®, compared with that in presence of IgG1 control, at E:T ratio of 1:1 (FIG. 4B). 6.5. Example 5: Antitumor Efficacy of PNK cells in a Glioblastoma Mouse Model

The anti-tumor efficacy of PNK cells was compared to vehicle controls in an orthotopic xenograft U87-Fluc glioblastoma model in NSG mice. The U87 cells used in this study were modified to express the firefly luciferase gene (U87-FLuc; PerkinElmer).

NSG mice were implanted with human U87-FLuc tumor cells orthotopically into the right striatum on Day 0. PNK cells were administered on Day 7 after the tumor implantation. Three different routes of administration of PNK cells or vehicle were evaluated:

Intracranial injection of 0.5×106 PNK into the right striatum (IC)

Intracerebroventricular injection of 1×106 PNK into the right ventricle (ICV)

Intravenous injection of 1×107 PNK into the tail vein (IV)

Treatment with PNK cells delivered via the IC route resulted in lower average tumor load in the treated mice on Day 26 after the tumor implantation as compared to the vehicle control. No differences in tumor growth were observed between PNK and corresponding vehicle-treated mice, when treatment was delivered via ICV or IV routes of administration. No adverse effects of treatments were observed from the day of tumor implantation (Day 0) until Day 28. By Day 29 mice showed clinical signs of declining health and loss of body weight, which continued until the end of the study on Day 35 and was consistent with tumor progression.

Forty-two NSG mice were injected intracranially (IC) into the right striatum with 1×104 U87-Fluc cells using Stoelting's stereotaxic instrument 51730D on Day 0. The coordinates used for IC injections were anterior-posterior (AP)=+2.0, medial-lateral (ML)=−1.5 and dorsal-ventral (DV)=−2.5. On Day 6, bioluminescent imaging (BLI) was performed and mice were randomized into treatment groups based on BLI values. Six mice were excluded and PBS/2 mM EDTA or PNK cells in PBS/2 mM EDTA were administered to 36 mice on Day 7 via three different routes: IC, ICV or IV, as shown in Table 1. The coordinates used for ICV injections were anterior-posterior (AP)=−0.4, medial-lateral (ML)=−1.0 and dorsal-ventral (DV)=−2.5. The coordinates used for IC injections were anterior-posterior (AP)=+2.0, medial-lateral (ML)=−1.5 and dorsal-ventral (DV)=−2.5. Body weights and BLI measurements were measured twice per week until Day 35. On Day 35, the mice were euthanized and plasma was collected. The brains of three (3) mice per group were snap frozen in liquid nitrogen and the remaining brains fixed in 4% PF A for histopathology. Brains from mice that lost 20% of their initial body weight, were moribund or euthanized at the request of the Sponsor prior to Day 35 were fixed in 4% PF A for histopathology.

On Day 0 of the study, U87-FLuc cells were suspended in PBS to give a final concentration of 1×10⁴ cells in 5 μL of sterile PBS. Cells were administered via an intracranial injection into the right striatum using Stoelting's stereotaxic instrument 51730D. The coordinates used for IC injections were anterior-posterior (AP)=+2.0, medial-lateral (ML)=−1.5 and dorsal-ventral (DV)=−2.5. The viability of the cells determined by trypan blue staining was 97%.

PNK cells were received frozen on dry ice and stored in liquid nitrogen. The cells were thawed at 37° C. and gently mixed. The contents were centrifuged for 7 minutes at 300×g and washed twice in PBS, reconstituted in 1 mL PBS/2 mM EDTA, counted using trypan blue and resuspended in sterile PBS/2 mM EDTA for immediate injection. The percent viability was assessed by trypan blue for each vial of cells used.

Mice in Groups 1 and 2 received 0.5×106 PNK cells in 5 μL of sterile PBS/2 mM EDTA or 5 μL PBS/2 mM EDTA via IC injection; the viability of the PNK cells determined by trypan blue staining was 87%. Groups 3 and 4 received 3×10⁶ PNK cells in 10 μL of sterile PBS/2 mM EDTA or 10 μL PBS/2 mM EDTA via ICV injection, and Groups 5 and 6 received 1×10⁷ PNK cells in 400 μL of sterile PBS/2 mM EDTA or 400 μL PBS/2 mM EDTA via IV injection. The viability of the PNK cells determined by trypan blue staining was 77%.

Body weights were recorded on Day 0 and on each day that BLI measurements were acquired. Mice were observed for clinical signs of ill health when body weights were taken. Bioluminescence was measured in mice lightly anesthetized with isoflurane using the IVIS Spectrum imager. Mice received 150 mg/kg of D-Luciferin (Perkin Elmer#122799) in PBS via an IP injection 25 minutes prior to image acquisition of the dorsal region of the head. IVIS imaging was performed on Days 0, 7, 12, 16, 19, 23, 26, 30 and 33 for Groups 1 and 2 and on Days 0, 6, 11, 14, 18, 21, 25, 28 and 32 for Groups 3 through 6.

The health status of the animals was checked daily. Clinical observations were recorded if any signs of declining health were observed. Dates of euthanasia or animals found dead were recorded.

Mice were euthanized on Day 35, brains were harvested for histopathology in 4% PFA or flash frozen, and blood samples were obtained via cardiac puncture and placed in K2EDTA tubes. Plasma was prepared via centrifugation, and stored at −80° C. When mice lost 20% of their initial body weight or were moribund before Day 35, they were euthanized and their brains were collected and fixed in 4% PF A for histopathology.

BLI and body weight data were analyzed using a two-way repeated measures ANOVA with Bonferroni's post hoc test and survival curves were analyzed using the Mantel-Cox log-rank test using GraphPad Prism 5.02.

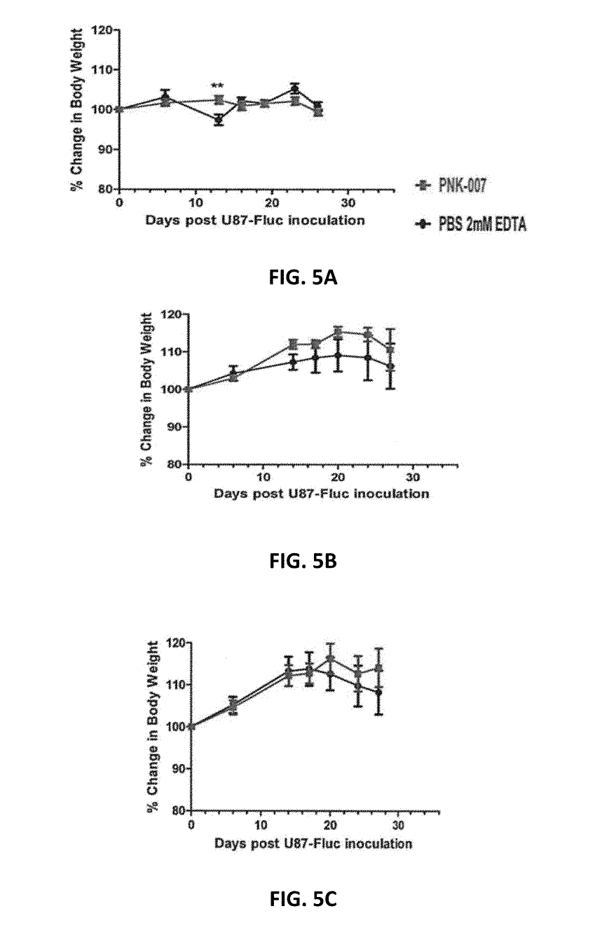

Body weights normalized to initial body weights measured on Day 0 are shown in FIGS. 5A, 5B and 5C. The body weights of mice that received IC injections of PNK cells were analyzed from Day 0 through Day 26 did not differ significantly from the body weights of mice treated with vehicle with respect to treatment effect (P=0.5416); however, the interaction between time and treatment was significant (P=0.0072). The post hoc analysis identified a significant difference on Day 13 as shown in FIG. 5A. The body weights of mice that received ICV and IV injections of PNK cells were analyzed from Day 0 through Day 27. Body weights appeared to increase more in the ICV group treated with PNK cells versus the vehicle control group and showed a trend towards significance due to treatment (P=0.0712, effect due to treatment and P=0.9062, effect due to the interaction between time and treatment). In the groups treated via IV injections no significant differences in body weight between the PNK treated and control groups were detected by the two-way ANOV A.

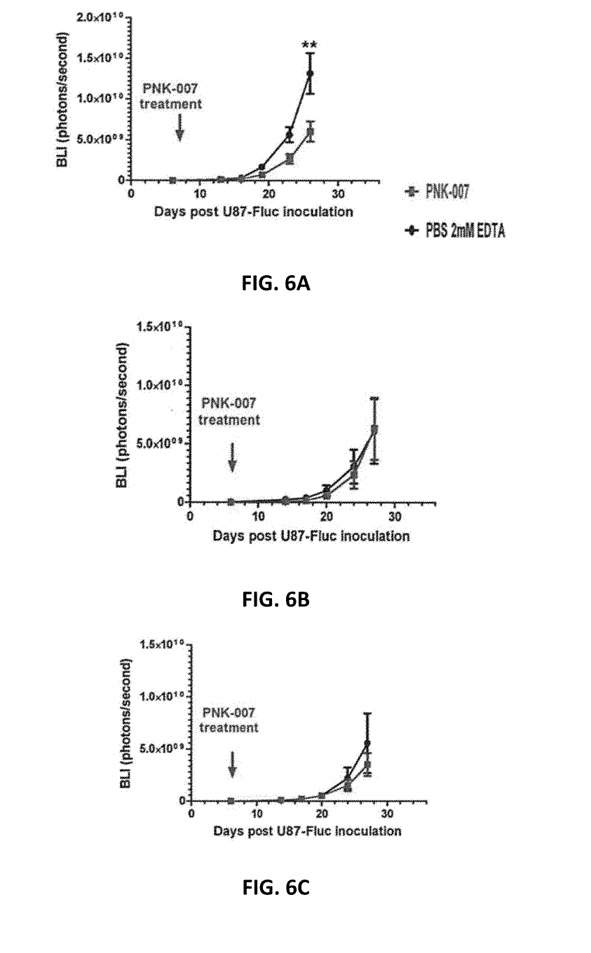

Bioluminescent imaging (BLI) data are plotted and analyzed from Day 0 (the tumor inoculation day) to Day 26 (IC) or Day 27 (ICV and IV). The data are shown in FIGS. 6A, 6B, and 6C. The tumor growth measured by BLI was reduced in the group that received PNK cells via IC injections, as compared to the corresponding control (P=0.0205, treatment and P<0.0001, time and treatment interaction). The post hoc analysis identified a significant difference on Day 26. No statistically significant differences in tumor growth were identified in mice that received PNK treatment via the ICV or IV routes of administration, as compared to the corresponding controls.

All animals appeared healthy during the first three weeks of the study after tumor inoculation. Survival curves for each ROA compared to the corresponding vehicle treated controls did not show statistically significant differences.

In this study we observed that injections of PNK cells administered via IC, ICV or IV injections were well tolerated, as assessed by clinical observations and body weight measurements. No adverse effects of treatments were observed until Day 28. On Day 29 mice showed clinical signs of declining health and loss of body weight, which continued until the end of the study on Day 35 and was consistent with tumor growth progression. The signs of deteriorating health were observed in mice from both vehicle and PNK treated groups.

The U87-FLuc tumor growth measured by BLI was reduced in the group that received PNK cells via IC injections compared to the vehicle treated controls. The post hoc analysis identified a significant difference between the groups on Day 26. No statistically significant differences in tumor growth were identified in mice that received PNK treatment via ICV or IV routes of administration, as compared to the corresponding controls.

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of treating a subject having glioblastoma by administering to the subject an effective amount of a cell population comprising human placenta-derived natural killer cells.

2. The method of claim 1, wherein the human placenta-derived natural killer cells are derived from umbilical cord blood, placental perfusate, or combinations thereof.

3. The method of claim 1, wherein the human placenta-derived natural killer cells are derived from umbilical cord blood.

4. The method of any one of claims 1-3, wherein the human placenta-derived natural killer cells are produced from hematopoietic stem cells.

5. The method of claim 4, wherein the hematopoietic stem cells are CD34+ hematopoietic stem cells.

6. The method of any one of claims 1-5, wherein the human placenta-derived natural killer cells are produced by a method comprising the steps of:
(a) culturing hematopoietic stem or progenitor cells in a first medium comprising a stem cell mobilizing agent and thrombopoietin (Tpo) to produce a first population of cells;
(b) culturing the first population of cells in a second medium comprising a stem cell mobilizing agent and interleukin-15 (IL-15), and lacking Tpo, to produce a second population of cells; and
(c) culturing the second population of cells in a third medium comprising IL-2 and IL-15, and lacking a stem cell mobilizing agent and LMWH, to produce a third population of cells.

7. The method of claim 6, wherein the third population of cells comprises natural killer cells that are CD56+, CD3−, CD16− or CD16+, and CD94+ or CD94−, and wherein at least 80% of the natural killer cells are viable.

8. The method of claim 6 or claim 7, wherein said Tpo is present in the first medium at a concentration of from 1 ng/mL to 50 ng/mL.

9. The method of claim 8, wherein said Tpo is present in the first medium at a concentration of from 20 ng/mL to 30 ng/mL.

10. The method of claim 8, wherein said Tpo is present in the first medium at a concentration of about 25 ng/mL.

11. The method of any one of claims 6-10, wherein said IL-15 is present in said second medium at a concentration of from 1 ng/mL to 50 ng/mL.

12. The method of any one of claims 6-10, wherein said IL-15 is present in said second medium at a concentration of from 10 ng/mL to 30 ng/mL.

13. The method of any one of claims 6-10, wherein said IL-15 is present in said second medium at a concentration of about 20 ng/mL.

14. The method of any one of claims 6-10, wherein said IL-2 is present in said third medium at a concentration of from 10 U/mL to 10,000 U/mL and said IL-15 is present in said third medium at a concentration of from 1 ng/mL to 50 ng/mL.

15. The method of any one of claims 6-10, wherein said IL-2 is present in said third medium at a concentration of from 300 U/mL to 3,000 U/mL and said IL-15 is present in said third medium at a concentration of from 10 ng/mL to 30 ng/mL.

16. The method of any one of claims 6-10, wherein said IL-2 is present in said third medium at a concentration of about 1,000 U/mL and said IL-15 is present in said third medium at a concentration of about 20 ng/mL.

17. The method of any of claims 6-16, wherein said Tpo, IL-2, and IL-15 are not comprised within an undefined component of the first medium, second medium or third medium.

18. The method of any of claims 6-16, wherein said Tpo, IL-2, and IL-15 are not comprised within serum.

19. The method of any of claims 6-16, wherein said stem cell mobilizing agent is an aryl hydrocarbon receptor inhibitor.

20. The method of claim 19, wherein said aryl hydrocarbon receptor inhibitor is StemRegenin-1 (SR-1) (4-(2-(2-(benzo[b]thiophen-3-yl)-9-isopropyl-9H-purin-6-ylamino)ethyl)phenol).

21. The method of claim 19, wherein said aryl hydrocarbon receptor inhibitor is resveratrol.

22. The method of claim 19, wherein said aryl hydrocarbon receptor inhibitor is the compound CH223191 (1-Methyl-N-[2-methyl-4-[2-(2-methylphenyl)diazenyl]phenyl-1H-pyrazole-5-carboxamide].

23. The method of any claims 1-18, wherein the stem cell mobilizing agent is a pyrimido(4,5-b)indole derivative.

24. The method of claim 23, wherein said pyrimido(4,5-b)indole derivative has the chemical structure:

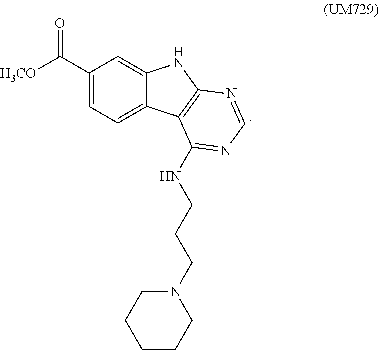 [SMILES: COC(=O)c1ccc2c(c1)[nH]c1ncnc(NCCCN3CCCCC3)c12]

25. The method of claim 24, wherein said pyrimido(4,5-b)indole derivative has the chemical structure

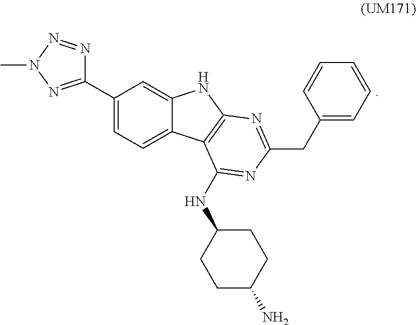 [SMILES: Cn1nnc(-c2ccc3c(c2)[nH]c2nc(Cc4ccccc4)nc(N[C@H]4CC[C@H](N)CC4)c23)n1]

26. The method of any of claims 6-25, wherein said first medium additionally comprises one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF).

27. The method of claim 26, wherein said first medium comprises each of LMWH, Flt-3L, SCF, IL-6, IL-7, G-CSF, and GM-CSF.

28. The method of claim 26 or claim 27, wherein in the first medium the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL.

29. The method of claim 26 or claim 27, wherein in the first medium the LMWH is present in the first medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL.

30. The method of claim 26 or claim 27, wherein in the first medium the LMWH is present in the first medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL.

31. The method of any of claims 1-30, wherein said second medium additionally comprises one or more of LMWH, Flt-3, SCF, IL-6, IL-7, G-CSF, and GM-CSF.

32. The method of any of claims 1-31, wherein said second medium additionally comprises each of LMWH, Flt-3, SCF, IL-6, IL-7, G-CSF, and GM-CSF.

33. The method of claim 31 or claim 32, wherein in the second medium the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL.

34. The method of claim 31 or claim 32, wherein in the second medium the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL.

35. The method of claim 31 or claim 32, wherein in the second medium the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL.

36. The method of any of claims 1-35, wherein said third medium additionally comprises one or more of SCF, IL-6, IL-7, G-CSF, or GM-CSF.

37. The method of claim 36, wherein said third medium comprises each of SCF, IL-6, IL-7, G-CSF, and GM-CSF.

38. The method of claim 36 or claim 37, wherein in the third medium the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL.

39. The method of claim 36 or claim 37, wherein in the third medium the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL.

40. The method of claim 36 or claim 37, wherein in the third medium the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL.

41. The method of any of claims 26-40, wherein said LMWH, Flt-3, SCF, IL-6, IL-7, G-CSF, and/or GM-CSF are not comprised within an undefined component of the first medium, second medium or third medium.

42. The method of any of claims 26-40, wherein said LMWH, Flt-3, SCF, IL-6, IL-7, G-CSF, and/or GM-CSF are not comprised within serum.

43. The method of any of claims 6-42, wherein any of said first medium, second medium or third medium comprises human serum-AB.

44. The method of claim 43, wherein any of said first medium, second medium or third medium comprises 1% to 20% human serum-AB.

45. The method of claim 43, wherein any of said first medium, second medium or third medium comprises 5% to 15% human serum-AB.

46. The method of claim 43, wherein any of said first medium, second medium or third medium comprises about 10% human serum-AB.

47. The method of any of claims 6-46, wherein any of said first medium, second medium or third medium comprises 2-mercaptoethanol.

48. The method of any of claims 6-46, wherein any of said first medium, second medium or third medium comprises gentamycin.

49. The method of any of claims 6-48, wherein said method comprises culturing the hematopoietic stem cells in the first medium for 7-13 days.

50. The method of claim 49, wherein said method comprises culturing the hematopoietic stem cells in the first medium for 8-12 days.

51. The method of claim 49, wherein said method comprises culturing the hematopoietic stem cells in the first medium for about 10 days.

52. The method of any of claims 6-48, wherein said method comprises culturing said first population of cells in said second medium for 2-6 days.

53. The method of any of claims 6-48, wherein said method comprises culturing said first population of cells in said second medium for 3-5 days.

54. The method of any of claims 6-48, wherein said method comprises culturing said first population of cells in said second medium for about 4 days.

55. The method of any of claims 6-48, wherein said method comprises culturing said second population of cells in said third medium for 10-30 days.

56. The method of any of claims 6-48, wherein said method comprises culturing said second population of cells in said third medium for 15-25 days.

57. The method of any of claims 6-48, wherein said method comprises culturing said second population of cells in said third medium for about 21 days.

58. The method of any of claims 6-48, wherein said culturing in said first medium, second medium and third medium are all done under static culture conditions.

59. The method of any of claims 6-48, wherein said culturing in at least one of said first medium, second medium or third medium are done in a spinner flask.

60. The method of any of claims 6-48, wherein said culturing in said first medium and said second medium is done under static culture conditions, and said culturing in said third medium is done in a spinner flask.

61. The method of any of claims 6-60, wherein said hematopoietic cells are initially inoculated into said first medium from 1×10⁴ to 1×10⁵ cells/mL.

62. The method of claim 61, wherein said hematopoietic cells are initially inoculated into said first medium at about 3×10⁴ cells/mL.

63. The method of any of claims 6-62, wherein said first population of cells are initially inoculated into said second medium from 5×10⁴ to 5×10⁵ cells/mL.

64. The method of any of claim 63, wherein said first population of cells is initially inoculated into said second medium at about 1×10⁵ cells/mL.

65. The method of any of claims 1-60, wherein said second population of cells is initially inoculated into said third medium from 1×10⁵ to 5×10⁶ cells/mL.

66. The method of claim 65, wherein said second population of cells is initially inoculated into said third medium from 1×10⁵ to 1×10⁶ cells/mL.

67. The method of claim 65, wherein said second population of cells is initially inoculated into said third medium at about 5×10⁵ cells/mL.

68. The method of claim 65, wherein said second population of cells is initially inoculated into said third medium at about 3×10⁵ cells/mL.

69. The method of any of claims 6-68, wherein said method produces at least 5000-fold more natural killer cells as compared to the number of hematopoietic stem cells initially inoculated into said first medium.

70. The method of claim 69, wherein said method produces at least 10,000-fold more natural killer cells.

71. The method of claim 69, wherein said method produces at least 50,000-fold more natural killer cells.

72. The method of claim 69, wherein said method produces at least 75,000-fold more natural killer cells.

73. The method of any of claims 6-68, wherein said method produces natural killer cells that comprise at least 20% CD56+CD3− natural killer cells.

74. The method of any of claims 6-68, wherein said method produces natural killer cells that comprise at least 40% CD56+CD3− natural killer cells.

75. The method of any of claims 6-68, wherein said method produces natural killer cells that comprise at least 60% CD56+CD3− natural killer cells.

76. The method of any of claims 6-68, wherein said method produces natural killer cells that comprise at least 80% CD56+CD3− natural killer cells.

77. The method of any of claims 6-68, wherein said natural killer cells exhibit at least 20% cytotoxicity against K562 cells when said natural killer cells and said K562 cells are co-cultured in vitro at a ratio of 10:1.

78. The method of claim 77, wherein said natural killer cells exhibit at least 35% cytotoxicity against the K562 cells.

79. The method of claim 77, wherein said natural killer cells exhibit at least 45% cytotoxicity against the K562 cells.

80. The method of claim 77, wherein said natural killer cells exhibit at least 60% cytotoxicity against the K562 cells.

81. The method of claim 77, wherein said natural killer cells exhibit at least 75% cytotoxicity against the K562 cells.

82. The method of any of claims 6-81, wherein viability of said natural killer cells is determined by 7-aminoactinomycin D (7AAD) staining.

83. The method of any of claims 6-81, wherein viability of said natural killer cells is determined by annexin-V staining.

84. The method of any of claims 6-81, wherein viability of said natural killer cells is determined by both 7-AAD staining and annexin-V staining.

85. The method of any of claims 6-81, wherein viability of said natural killer cells is determined by trypan blue staining.

86. The method of any of claims 6-85, wherein the human placenta-derived natural killer cells are produced by a method additionally comprising the step of cryopreserving said population of cells after step (c).

87. The method of claim 86, wherein said cryopreserved cell population is administered to the subject within about six hours after thawing.

88. The method of any of claims 6-85, wherein the human placenta-derived natural killer cells are not cryopreserved.

89. The method of any one of claims 1-88, wherein the subject is a mammal.

90. The method of any one of claims 1-88, wherein the subject is a human.

91. The method of any one of claims 1-90, wherein the treating further comprises administering to the subject an effective amount of an additional anti-cancer treatment.

92. The method of claim 91, wherein the additional anti-cancer treatment is selected from the group consisting of radiation therapy, chemotherapy, antibody-based therapy, and combinations thereof.

93. The method of any one of claims 1-92, wherein the treating further comprises administering to the subject an effective amount of an anticonvulsant.

94. The method of any one of claims 1-93, wherein the treating further comprises administering to the subject an effective amount of a corticosteroid.

95. The method of any one of claims 1-94, wherein said subject is administered about 1×10⁴, 3×10⁴, 1×10⁵, 3×10⁵, 1×10⁶, 3×10⁶, 1×10⁷, 3×10⁷, 1×10⁸, or 3×10⁸ natural killer cells per kilogram of the subject.

96. The method of any one of claims 1-95, wherein the administration is intracranial, (IC), intracerebral ventricular (ICV), or intraveinous (IV).

97. The method of any one of claims 1-96, wherein the treatment comprises administration of more than one dose of the cell population comprising human placenta-derived natural killer cells.

98. The method of claim 97, wherein the treatment comprises administration of two, three, four, or more doses of the cell population comprising human placenta-derived natural killer cells.

99. The method of any one of claims 1-98, wherein the natural killer cells are genetically modified.

100. A method of treating a subject having a brain tumor by administering to the subject an effective amount of a cell population comprising human placenta-derived natural killer cells.

101. A method of suppressing the growth of brain tumor cells comprising contacting the glioblastoma cells with an effective amount of a cell population comprising human placenta-derived natural killer cells.

102. The method of claim 101, wherein said contacting takes place in vitro.

103. The method of claim 101, wherein said contacting takes place in vivo.

104. The method of claim 101, wherein said contacting takes place in a human individual.

105. The method of claim 101, wherein said method comprises administering said natural killer cells to said individual.

106. A composition comprising subject an effective amount of a cell population comprising human placenta-derived natural killer cells for use in the treatment of a brain tumor in a subject.

107. Use of a composition comprising subject an effective amount of a cell population comprising human placenta-derived natural killer cells in the treatment of a brain tumor in a subject.

108. Use of a composition comprising subject an effective amount of a cell population comprising human placenta-derived natural killer cells in the manufacture of a a brain tumor for treatment of glioblastoma in a subject.

109. The composition of claim 106 or the use of claim 107 or claim 108, wherein the brain tumor is a glioblastoma.
